(19) United States
(12) Patent Application Publication
(10) Pub. No.: US 2007/0259849 A1
(43) Pub. Date: Nov. 8, 2007

(54) Azine-Carboxamides as Anti-Cancer Agents

(75) Inventors: Brian Aquila, Marlborough, MA (US); Stephanos Ioannidis, Cambridge, MA (US); Paul Lyne, Arlington, MA (US); Timothy Pontz, Cambridge, MA (US)

Correspondence Address: ASTRAZENECA R&D BOSTON, 35 GATEHOUSE DRIVE, WALTHAM, MA, 02451-1215, US

(73) Assignee: ASTRAZENECA AB, Sodertalje (SE)

(21) Appl. No.: 11/570,065

(22) PCT Filed: Jun. 29, 2005
(86) PCT No.: PCT/GB05/02522

§ 371 Date: Dec. 5, 2006

Related U.S. Application Data
(60) Provisional application No. 60/584,129, filed on Jul. 1, 2004.

(51) Int. Cl. C07D 239/42; A61K 31/435; A61K 31/495; A61P 35/00; C07D 241/24; C07D 403/04; C07D 405/12; C07D 403/06; C07D 401/04; C07D 213/81; C07D 213/82; C07D 239/28

(52) U.S. Cl. 514/211.05; 514/235.5; 514/235.8; 514/252.14; 514/255.05; 514/256; 514/318; 514/326; 514/354; 514/357; 540/507; 544/121; 544/124; 544/242; 544/357; 544/405; 546/193; 546/276.4; 546/336; 546/340

(57) ABSTRACT

The invention relates to chemical compounds, of the formula (I): or pharmaceutically acceptable salts thereof, which possess B-Raf inhibitory activity and are accordingly useful for their anti cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm blooded animal such as man.

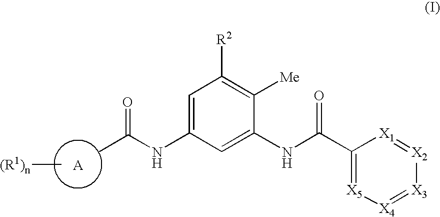

The invention relates to chemical compounds, or pharmaceutically acceptable salts thereof, which possess B-Raf inhibitory activity and are accordingly useful for their anti-cancer activity and thus in methods of treatment of the human or animal body. The invention also relates to processes for the manufacture of said chemical compounds, to pharmaceutical compositions containing them and to their use in the manufacture of medicaments of use in the production of an anti-cancer effect in a warm-blooded animal such as man.

The classical Ras, Raf, MAP protein kinase/extracellular signal-regulated kinase kinase (MEK), extracellular signal-regulated kinase (ERK) pathway plays a central role in the regulation of a variety of cellular functions dependent upon cellular context, including cellular proliferation, differentiation, survival, immortalization and angiogenesis (reviewed in Peyssonnaux and Eychene, Biology of the Cell, 2001, 93, 3-62). In this pathway, Raf family members are recruited to the plasma membrane upon binding to guanosine triphosphate (GTP) loaded Ras resulting in the phosphorylation and activation of Raf proteins. Activated Rafs then phosphorylate and activate MEKs, which in turn phosphorylate and activate ERKs. Upon activation, ERKs translocate from the cytoplasm to the nucleus resulting in the phosphorylation and regulation of activity of transcription factors such as Elk-1 and Myc.

The Ras/Raf/MEK/ERK pathway has been reported to contribute to the tumorigenic phenotype by inducing immortalisation, growth factor-independent growth, insensitivity to growth-inhibitory signals, ability to invade and metastasis, stimulating angiogenesis and inhibition of apoptosis (reviewed in Kolch et al., Exp. Rev. Mol. Med., 2002, 25 April, http://www.expertreviews.org/02004386h.htm). In fact, ERK phosphorylation is enhanced in approximately 30% of all human tumours (Hoshino et al., Oncogene, 1999, 18, 813-822). This may be a result of overexpression and/or mutation of key members of the pathway.

Three Raf serine/threonine protein kinase isoforms have been reported Raf-1/c-Raf, B-Raf and A-Raf (reviewed in Mercer and Pritchard, Biochim. Biophys. Acta, 2003, 1653, 25-40), the genes for which are thought to have arisen from gene duplication. All three Raf genes are expressed in most tissues with high-level expression of B-Raf in neuronal tissue and A-Raf in urogenital tissue. The highly homologous Raf family members have overlapping but distinct biochemical activities and biological functions (Hagemarn and Rapp, Expt. Cell Res. 1999, 253, 34-46). Expression of all three Raf genes is required for normal murine development however both c-Raf and B-Raf are required to complete gestation. B-Raf−/− mice die at E12.5 due to vascular hemorrhaging caused by increased apoptosis of endothelial cells (Wojnowski et al., Nature Genet., 1997, 16, 293-297). B-Raf is reportedly the major isoform involved in cell proliferation and the primary target of oncogenic Ras. Activating somatic missense mutations have been identified exclusively for B-Raf, occurring with a frequency of 66% in malignant cutaneous melanomas (Davies et al., Nature, 2002, 417, 949-954) and also present in a wide range of human cancers, including but not limited to papillary thyroid tumours (Cohen et al., J. Natl. Cancer Inst., 2003, 95, 625-627), cholangiocarcinomas (Tannapfel et al., Gut, 2003, 52, 706-712), colon and ovarian cancers (Davies et al., Nature, 2002, 417, 949-954). The most frequent mutation in B-Raf (80%) is a glutamic acid for valine substitution at position 600. These mutations increase the basal kinase activity of B-Raf and are thought to uncouple Raf/MEK/ERK signalling from upstream proliferation drives including Ras and growth factor receptor activation resulting in constitutive activation of ERK. Mutated B-Raf proteins are transforming in NIH3T3 cells (Davies et al., Nature, 2002, 417, 949-954) and melanocytes (Wellbrock et al., Cancer Res., 2004, 64, 2338-2342) and have also been shown to be essential for melanoma cell viability and transformation (Hingorani et al., Cancer Res., 2003, 63, 5198-5202). As a key driver of the Raf/MEK/ERK signalling cascade, B-Raf represents a likely point of intervention in tumours dependent on this pathway.

AstraZeneca applications WO 00/18738 and WO 00/07991 disclose certain benzene-1,3-aminocarbonyl compounds which are inhibitors of the production of cytokines such as TNF, in particular of TNFα, and various interleukins, in particular IL-1. The present inventors have surprisingly found that certain other, novel, benzene-1,3-aminocarbonyl compounds are potent B-Raf inhibitors and are accordingly expected to be useful in the treatment of neoplastic disease.

Accordingly, the present invention provides a compound of formula (I): 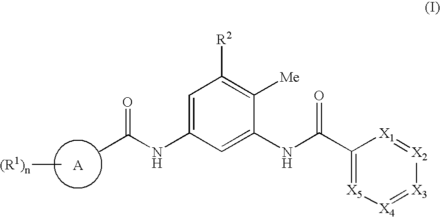
wherein:

Ring A is carbocyclyl or heterocyclyl; wherein if said heterocyclyl contains an —NH— moiety that nitrogen may be optionally substituted by a group selected from R³;

R¹ is a substituent on carbon and is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁴— or heterocyclyl-R⁵—; wherein R¹ may be optionally substituted on carbon by one or more R⁶; and wherein if said heterocyclyl contains an —NH— moiety that nitrogen may be optionally substituted by a group selected from R⁷;

R² is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁸— or heterocyclyl-R⁹—; wherein R² may be optionally substituted on carbon by one or more R¹⁰; and wherein if said heterocyclyl contains an —NH— moiety that nitrogen may be optionally substituted by a group selected from R¹¹;

X₁ is N and X₂, X₃, X₄ and X₅ are independently CR¹²; or two X₁, X₂, X₃, X₄ and X₅ are N; the other X₁, X₂, X₃, X₄ and X₅ are independently CR¹²;

n is selected from 0-4; wherein the values of R¹ may be the same or different;

R⁶ and R¹⁰ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹³— or heterocyclyl-R¹⁴—; wherein R⁶ and R¹⁰ independently of each other may be optionally substituted on carbon by one or more R¹⁵; and wherein if said heterocyclyl contains an —NH— moiety that nitrogen may be optionally substituted by a group selected from R¹⁶;

R¹² is independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R¹⁷— or heterocyclyl-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;

R¹⁹ is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R²¹— or heterocyclyl-R²²—; wherein R¹⁹ may be optionally substituted on carbon by one or more R²³; and wherein if said heterocyclyl contains an —NH— moiety that nitrogen may be optionally substituted by a group selected from R²⁴;

R⁴, R⁵, R⁸, R⁹, R¹³, R¹⁴, R¹⁷, R¹⁸, R²¹ and R²² are independently selected from a direct bond, —O—, —N(R²⁵)—, —C(O)—, —N(R²⁶)C(O)—, —C(O)N(R²⁷)—, —S(O)ₛ—, —SO₂N(R²³)— or —N(R²⁹)SO₂—; wherein R²⁵, R²⁶, R²⁷, R²⁸ and R²⁹ are independently selected from hydrogen or C₁₋₆alkyl and s is 0-2;

R³, R⁷, R¹¹, R¹⁶, R²⁰ and R²⁴ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R¹⁵ and R²³ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;

with the proviso that said compound is not 4-amino-2-(methylthio)-N-(2-methyl-5-{[3-(trifluoromethyl)benzoyl]amino}phenyl)pyrimidine-5-carboxamide.

According to a further aspect of the invention there is provided the use of a compound of the formula (I), as depicted above, wherein:

Ring A is carbocyclyl or heterocyclyl; wherein if said heterocyclyl contains an —NH— moiety that nitrogen may be optionally substituted by a group selected from R³;

R¹ is a substituent on carbon and is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁴— or heterocyclyl-R⁵—; wherein R¹ may be optionally substituted on carbon by one or more R⁶; and wherein if said heterocyclyl contains an —NH— moiety that nitrogen may be optionally substituted by a group selected from R⁷;

R² is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₁₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁸— or heterocyclyl-R⁹—; wherein R² may be optionally substituted on carbon by one or more R¹⁰; and wherein if said heterocyclyl contains an —NH— moiety that nitrogen may be optionally substituted by a group selected from R¹¹;

one or two X₁, X₂, X₃, X₄ and X₅ are N; the other X₁, X₂, X₃, X₄ and X₅ are independently CR¹²;

n is selected from 0-4; wherein the values of R¹ may be the same or different;

R⁶ and R¹⁰ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹³— or heterocyclyl-R¹⁴—; wherein R⁶ and R¹⁰ independently of each other may be optionally substituted on carbon by one or more R¹⁵; and wherein if said heterocyclyl contains an —NH— moiety that nitrogen may be optionally substituted by a group selected from R¹⁶;

R¹² is independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R¹⁷— or heterocyclyl-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;

R¹⁹ is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R²¹— or heterocyclyl-R²²—; wherein R¹⁹ may be optionally substituted on carbon by one or more R²³; and wherein if said heterocyclyl contains an —NH— moiety that nitrogen may be optionally substituted by a group selected from R²⁴;

R⁴, R⁵, R⁸, R⁹, R¹³, R¹⁴, R¹⁷, R¹⁸, R²¹ and R²² are independently selected from a direct bond, —O—, —N(R²⁵)—, —C(O)—, —N(R²⁶)C(O)—, —C(O)N(R²⁷)—, —S(O)S—, —SO₂N(R²⁸)— or —N(R²⁹)SO₂—; wherein R²⁵, R²⁶, R²⁷, R²⁸ and R²⁹ are independently selected from hydrogen or C₁₋₆alkyl and s is 0-2;

R³, R⁷, R¹¹, R¹⁶, R²⁰ and R²⁴ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;

R¹⁵ and R²³ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof;

in the manufacture of a medicament for use in the production of a B-Raf inhibitory effect in a warm-blooded animal such as man.

In a further aspect of the invention there is provided a compound of formula (I), as depicted above, wherein:

Ring A is carbocyclyl or heterocyclyl; wherein if said heterocyclyl contains an —NH— moiety that nitrogen may be optionally substituted by a group selected from R³;

R¹ is a substituent on carbon and is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁴— or heterocyclyl-R⁵—; wherein R¹ may be optionally substituted on carbon by one or more R⁶; and wherein if said heterocyclyl contains an —NH— moiety that nitrogen may be optionally substituted by a group selected from R⁷;

R² is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁸— or heterocyclyl-R⁹—; wherein R² may be optionally substituted on carbon by one or more R¹⁰; and wherein if said heterocyclyl contains an —NH— moiety that nitrogen may be optionally substituted by a group selected from R¹¹;

one or two X₁, X₂, X₃, X₄ and X₅ are N; the other X₁, X₂, X₃, X₄ and X₅ are CR¹²;

n is selected from 0-4; wherein the values of R¹ may be the same or different;

R⁶ and R¹⁰ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹³— or heterocyclyl-R¹⁴—; wherein R⁶ and R¹⁰ independently of each other may be optionally substituted on carbon by one or more R⁵; and wherein if said heterocyclyl contains an —NH— moiety that nitrogen may be optionally substituted by a group selected from R¹⁶;

R¹² is independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹⁷— or heterocyclyl-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;

R¹⁹ is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R²¹— or heterocyclyl-R²²—; wherein R¹⁹ may be optionally substituted on carbon by one or more R²³; and wherein if said heterocyclyl contains an —NH— moiety that nitrogen may be optionally substituted by a group selected from R²⁴;

R⁴, R⁵, R⁸, R⁹, R¹³, R¹⁴, R¹⁷, R¹⁸, R²¹ and R²² are independently selected from a direct bond, —O—, —N(R²⁵)—, —C(O)—, —N(R²⁶)C(O)—, —C(O)N(R²⁷)—, —S(O)ₛ—, —SO₂N(R²⁸)— or —N(R²⁹)SO₂—; wherein R²⁵, R²⁶, R²⁷, R²⁸ and R²⁹ are independently selected from hydrogen or C₁₋₆alkyl and s is 0-2;

R³, R⁷, R¹¹, R¹⁶, R²⁰ and R²⁴ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzyl and phenylsulphonyl;

R¹⁵ and R²³ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;

or a pharmaceutically acceptable salt thereof.

In this specification the term “alkyl” includes both straight and branched chain alkyl groups. References to individual alkyl groups such as “propyl” are specific for the straight chain version only and references to individual branched chain alkyl groups such as ‘isopropyl’ are specific for the branched chain version only. For example, “C₁₋₆alkyl” includes C₁₋₄alkyl, C₁₋₃alkyl, propyl, isopropyl and t-butyl. A similar convention applies to other radicals, for example “phenylC₁₋₆alkyl” includes phenylC₁₋₄alkyl, benzyl, 1-phenylethyl and 2-phenylethyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

A “heterocyclyl” is a saturated, partially saturated or unsaturated, mono or bicyclic ring containing 4-12 atoms of which at least one atom is chosen from nitrogen, sulphur or oxygen, which may, unless otherwise specified, be carbon or nitrogen linked, wherein a —CH₂— group can optionally be replaced by a —C(O)— and a ring sulphur atom may be optionally oxidised to form the S-oxides. Examples and suitable values of the term “heterocyclyl” are morpholino, piperidyl, pyridyl, pyranyl, pyrrolyl, pyrazolyl, isothiazolyl, indolyl, quinolyl, thienyl, 1,3-benzodioxolyl, thiadiazolyl, piperazinyl, thiazolidinyl, pyrrolidinyl, thiomorpholino, pyrrolinyl, homopiperazinyl, 3,5-dioxapiperidinyl, tetrahydropyranyl, imidazolyl, pyrimidyl, pyrazinyl, pyridazinyl, isoxazolyl, N-methylpyrrolyl, 4-pyridone, 1-isoquinolone, 2-pyrrolidone, 4-thiazolidone, pyridine-N-oxide and quinoline-N-oxide. Further examples and suitable values of the term “heterocyclyl” are piperidinyl, 1,4-oxazepanyl, tetrahydropyranyl, piperazinyl, imidazolyl, 2-oxopiperazinyl, 5-oxo-2,5-dihydro-1H-pyrazolyl, pyrazolyl, pyrrolidinyl, pyridinyl, 3,6-dihydropyridin(2H)-yl and morpholino. A particular example of the term “heterocyclyl” is pyrazolyl. In one aspect of the invention a “heterocyclyl” is a saturated, partially saturated or unsaturated, monocyclic ring containing 5 or 6 atoms of which at least one atom is chosen from nitrogen, sulphur or oxygen, it may, unless otherwise specified, be carbon or nitrogen linked, a —CH₂— group can optionally be replaced by a —C(O)— and a ring sulphur atom may be optionally oxidised to form the S-oxides.

A “carbocyclyl” is a saturated, partially saturated or unsaturated, mono or bicyclic carbon ring that contains 3-12 atoms; wherein a —CH₂— group can optionally be replaced by a —C(O)—. Particularly “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. A particular example of “carbocyclyl” is phenyl.

An example of “C₁₋₆alkanoyloxy” is acetoxy. Examples of “C₁₋₆alkoxycarbonyl” include methoxycarbonyl, ethoxycarbonyl, n- and t-butoxycarbonyl. Examples of “C₁₋₆alkoxy” include methoxy, ethoxy and propoxy. Examples of “C₁₋₆alkanoylamino” include formamido, acetamido and propionylamino. Examples of “C₁₋₆alkylS(O)ₐ wherein a is 0 to 2” include methylthio, ethylthio, methylsulphinyl, ethylsulphinyl, mesyl and ethylsulphonyl. Examples of “C₁₋₆alkanoyl” include propionyl and acetyl. Examples of “N—(C₁₋₆alkyl)amino” include methylamino and ethylamino. Examples of “N,N—(C₁₋₆alkyl)₂amino” include di-N-methylamino, di-(N-ethyl)amino and N-ethyl-N-methylamino. Examples of “C₂₋₆alkenyl” are vinyl, allyl and 1-propenyl. Examples of “C₂₋₆allynyl” are ethynyl, 1-propynyl and 2-propynyl. Examples of “N—(C₁₋₆alkyl)sulphamoyl” are N-(methyl)sulphamoyl and N-(ethyl)sulphamoyl. Examples of “N—(C₁₋₆alkyl)₂sulphamoyl” are N,N-(dimethyl)sulphamoyl and N-(methyl)-N-(ethyl)sulphamoyl. Examples of “N—(C₁₋₆alkyl)carbamoyl” are N—(C₁₋₄alkyl)carbamoyl, methylaminocarbonyl and ethylaminocarbonyl. Examples of “N,N—(C₁₋₆alkyl)₂carbamoyl” are N,N—(C₁₋₄alkyl)₂carbamoyl, dimethylaminocarbonyl and methylethylaminocarbonyl. Examples of “C₁₋₆alkylsulphonyl” are mesyl, ethylsulphonyl and isopropylsulphonyl. Examples of “C₁₋₆alkylsulphonylamino” are mesylamino, ethylsulphonylamino and isopropylsulphonylamino.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have chiral centres and/or geometric isomeric centres (E- and Z-isomers), and it is to be understood that the invention encompasses all such optical, diastereoisomers and geometric isomers that possess B-Raf inhibitory activity. The invention further relates to any and all tautomeric forms of the compounds of the formula (I) that possess B-Raf inhibitory activity.

It is also to be understood that certain compounds of the formula (I) can exist in solvated as well as unsolvated forms such as, for example, hydrated forms. It is to be understood that the invention encompasses all such solvated forms which possess B-Raf inhibitory activity.

Particular values of variable groups are as follows. Such values may be used where appropriate with any of the definitions, claims or embodiments defined hereinbefore or hereinafter.

Ring A is carbocyclyl.

Ring A is heterocyclyl; wherein if said heterocyclyl contains an —NH— moiety that nitrogen may be optionally substituted by a group selected from R³.

Ring A is phenyl.

R¹ is a substituent on carbon and is C₁₋₆alkyl; wherein R¹ may be optionally substituted on carbon by one or more R⁶.

R¹ is a substituent on carbon and is selected from C₁₋₆alkyl or C₁₋₆alkoxy; wherein R¹ may be optionally substituted on carbon by one or more R⁶; wherein R⁶ is selected from halo, cyano or heterocyclyl-R¹⁴—; and R¹⁴ is a direct bond.

R¹ is a substituent on carbon and is C₁₋₆alkyl; wherein R¹ may be optionally substituted on carbon by one or more R⁶; wherein R⁶ is selected from halo or cyano.

R¹ is a substituent on carbon and is selected from C₁₋₆alkyl or C₁₋₆alkoxy; wherein R¹ may be optionally substituted on carbon by one or more R⁶; wherein R⁶ is selected from fluoro, cyano or morpholino.

R¹ is a substituent on carbon and is C₁₋₆alkyl; wherein R¹ may be optionally substituted on carbon by one or more R⁶; wherein R⁶ is selected from fluoro or cyano.

R¹ is a substituent on carbon and is trifluoromethyl or 1-cyano-1-methylethyl.

R¹ is a substituent on carbon and is trifluoromethyl, 1-cyano-1-methylethyl or 2-(morpholino)ethoxy.

R¹ is a substituent on carbon and is 1-cyano-1-methylethyl.

R² is hydrogen.

X₁ is N; the other X₂, X₃, X₄ and X₅ are CR¹².

X₂ is N; the other X₁, X₃, X₄ and X₅ are CR¹².

X₃ is N; the other X₁, X₂, X₄ and X₅ are CR¹².

X₁ and X₂ are N; X₂, X₄ and X₅ are CR¹².

X₁ and X₃ are N; X₂, X₄ and X₅ are CR¹².

X₁ and X₄ are N; X₂, X₃ and X₅ are CR¹².

X₁ and X₅ are N; X₂, X₃ and X₄ are CR¹².

X₂ and X₃ are N; X₁, X₃ and X₅ are CR¹².

X₂ and X₄ are N; X₁, X₃ and X₅ are CR¹².

X₂ and X₅ are N; X₂, X₃ and X₅ are CR¹².

X₁ is N; the other X₂, X₃, X₄ and X₅ are CR¹²; or X₂ is N; the other X₁, X₃, X₄ and X₅ are CR¹²; or X₃ is N; the other X₁, X₂, X₄ and X₅ are CR¹²; or X₁ and X₃ are N; X₂, X₄ and X₅ are CR¹²; or X₁ and X₅ are N; X₂, X₃ and X₅ are CR¹² or X₁ and X₅ are N; X₂, X₃ and are CR¹²; or X₂ and X₄ are N; X₁, X₃ and X₅ are CR¹²; or X₂ and X₅ are N; X₁, X₃ and X₅ are CR¹²; wherein:

R¹² is independently selected from hydrogen, halo, cyano, amino, carboxy, carbamoyl, C₁₋₆alkyl, N—(C₁₋₆alkyl)amino, N,N—(C₁₋₆alkyl)₂amino, N—(C₁₋₆alkyl)carbamoyl, C₁₋₆alkylS(O)ₐ wherein a is 0, carbocyclyl-R¹⁷— or heterocyclyl-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;

R¹⁹ is selected from halo, cyano, hydroxy, amino, C₁₋₆alkyl, C₁₋₆alkoxy, N,N—(C₁₋₆alkyl)₂amino, C₁₋₆alkoxycarbonylamino or heterocyclyl-R²²—; wherein R¹⁹ may be optionally substituted on carbon by one or more R²³;

R¹⁷, R¹⁸ and R²² are independently selected from a direct bond, —N(R²⁵)— or —N(R²⁶)C(O)—; wherein R²⁵ and R²⁶ are independently selected from hydrogen;

R²⁰ is selected from C₁₋₆alkyl and C₁₋₆alkoxycarbonyl;

R²³ is hydroxy.

X₁ is N; the other X₂, X₃, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; or X₂ and X₅ are N; X₁, X₃ and X₅ are CR¹²; wherein:

R¹² is independently selected from hydrogen, halo, cyano, amino, carboxy, carbamoyl, C₁₋₆alkyl, N—(C₁₋₆alkyl)amino, N,N—(C₁₋₆alkyl)₂amino, N—(C₁₋₆alkyl)carbamoyl, C₁₋₆alkylS(O)ₐ wherein a is 0, carbocyclyl-R¹⁷— or heterocyclyl-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;

R¹⁹ is selected from halo, cyano, hydroxy, amino, C₁₋₆alkyl, C₁₋₆alkoxy, N,N—(C₁₋₆alkyl)₂amino, C₁₋₆alkoxycarbonylamino or heterocyclyl-R²²—; wherein R¹⁹ may be optionally substituted on carbon by one or more R²³;

R¹⁷, R¹⁸ and R²² are independently selected from a direct bond, —N(R²⁵)— or —N(R²⁶)C(O)—; wherein R²⁵ and R²⁶ are independently selected from hydrogen;

R²⁰ is selected from C₁₋₆alkyl and C₁₋₆alkoxycarbonyl;

R²³ is hydroxy.

X₁ is N; the other X₂, X₃, X₄ and X₅ are CR¹²; or X₂ is N; the other X₁, X₃, X₄ and X₅ are CR¹²; or X₃ is N; the other X₁, X₂, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; wherein

R¹² is independently selected from hydrogen, halo, C₁₋₆alkyl, N—(C₁₋₆alkyl)amino, N,N—(C₁₋₆alkyl)₂amino or heterocyclyl-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;

R¹⁹ is selected from halo, C₁₋₆alkyl, C₁₋₆alkoxy or heterocyclyl-R²²—;

R¹⁸ and R²² are independently selected from a direct bond or —N(R²⁵)—; wherein R²⁵ is selected from hydrogen;

R²⁰ is selected from C₁₋₆alkyl or C₁₋₆alkoxycarbonyl.

X₁ is N; the other X₂, X₃, X₄ and X₅ are CR¹²; or X₂ is N; the other X₁, X₃, X₄ and X₅ are CR²; or X₃ is N; the other X₁, X₂, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; or X₂ and X₅ are N; X₁, X₃ and X₅ are CR¹²; wherein:

R¹² is independently selected from hydrogen, chloro, bromo, cyano, amino, carboxy, carbamoyl, methyl, ethyl, N-methylamino, N-ethylamino, N,N-dimethylamino, N-methyl-N-ethylamino, N-methylcarbamoyl, methylthio, cyclopropyl-R¹⁷—, piperidin-1-yl-R¹⁸—, piperidin-4-yl-R¹⁸—, 1,4-oxazepan-4-yl-R¹⁸—, tetrahydropyran-4-yl-R¹⁸—, piperazin-4-yl-R¹¹—, imidazol-4-yl-R¹⁸—, imidazol-5-yl-R¹⁸, 2-oxopiperazin-4-yl-R¹⁸—, 5-oxo-2,5-dihydro-1H-pyrazol-3-yl-R¹—, pyrazol-4-yl-R¹⁸—, pyrrolidin-1-yl-R¹⁸—, pyridin-3-yl-R¹⁸—, pyridin-4-yl-R¹⁸—, 3,6-dihydropyridin-1(2H)-yl-R¹⁸— or morpholino-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;

R¹⁹ is selected from fluoro, cyano, hydroxy, amino, methyl, methoxy, N,N-dimethylamino, t-butoxycarbonylamino, imidazol-2-yl-R²²— or pyrrolidin-1-yl-R²²—; wherein R¹⁹ may be optionally substituted on carbon by one or more R²³;

R¹⁷, R¹⁸ and R²² are independently selected from a direct bond, —N(R²⁵)— or —N(R²⁶)C(O)—; wherein R²⁵ and R²⁶ are independently selected from hydrogen;

R²⁰ is selected from methyl and t-butoxycarbonyl;

R²³ is hydroxy.

X₁ is N; the other X₂, X₃, X₄ and X₅ are CR¹²; or X₁ and X₃ are N; X₂, X₄ and X₅ are CR¹²; or X₁ and X₄ are N; X₂, X₃ and X₄ are CR¹²; or X₁ and X₅ are N; X₂, X₃ and X₄ are CR¹²; or X₂ and X₄ are N; X₁, X₃ and X₅ are CR¹²; or X₂ and X₅ are N; X₁, X₃ and X₅ are CR¹²; wherein:

R¹² is independently selected from hydrogen, chloro, bromo, cyano, amino, carboxy, carbamoyl, methyl, ethyl, N-methylamino, N-ethylamino, N,N-dimethylamino, N-methyl-N-ethylamino, N-methylcarbamoyl, methylthio, cyclopropyl-R¹⁷—, piperidin-1-yl-R¹⁸—, piperidin-4-yl-R¹⁸—, 1,4-oxazepan-4-yl-R¹⁸—, tetrahydropyran-4-yl-R¹⁸—, piperazin-4-yl-R¹⁸—, imidazol-4-yl-R¹⁸—, imidazol-5-yl-R¹⁸—, 2-oxopiperazin-4-yl-R¹⁸—, 5-oxo-2,5-dihydro-1H-pyrazol-3-yl-R¹⁸—, pyrazol-4-yl-R¹⁸—, pyrrolidin-1-yl-R¹⁸—, pyridin-3-yl-R¹⁸—, pyridin-4-yl-R¹⁸—, 3,6-dihydropyridin-1(2H)-yl-R¹⁸ or morpholino-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;

R¹⁹ is selected from fluoro, cyano, hydroxy, amino, methyl, methoxy, N,N-dimethylamino, t-butoxycarbonylamino, imidazol-2-yl-R²²— or pyrrolidin-1-yl-R²²—; wherein R¹⁹ may be optionally substituted on carbon by one or more R²³;

R¹⁷, R¹⁸ and R²² are independently selected from a direct bond, —N(R²⁵)— or —N(R²⁶)C(O)—; wherein R²⁵ and R¹⁶ are independently selected from hydrogen;

R²⁰ is selected from methyl and t-butoxycarbonyl;

R²³ is hydroxy.

X₁ is N; the other X₂, X₃, X₄ and X₅ are CR¹²; or X₂ is N; the other X₁, X₃, X₄ and X₅ are CR¹²; or X₃ is N; the other X₁, X₂, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; wherein

R¹² is independently selected from hydrogen, chloro, methyl, ethyl, methylamino, N-methyl-N-ethylamino, morpholino, piperazin-1-yl, 3-oxopiperazin-1-yl, piperidin-1-yl, 1,4-oxazepan-4-yl or tetrahydropyran-4-ylamino; wherein R¹² independently of each other may be optionally substituted on carbon by one or more R¹⁹; and wherein any piperazin-1-yl may be optionally substituted by a group selected from R²⁰;

R¹⁹ is selected from fluoro, hydroxy, methyl, methoxy or pyrrolidin-1-yl;

R²⁰ is selected from methyl or t-butoxycarbonyl.

X₁ is N; the other X₂, X₃, X₄ and X₅ are CR¹²; or X₂ is N; the other X₁, X₃, X₄ and X₅ are CR¹²; or X₃ is N; the other X₁, X₂, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; or X₂ and X₅ are N; X₁, X₃ and X₅ are CR¹²; wherein:

R¹² is independently selected from hydrogen, chloro, bromo, cyano, amino, carboxy, carbamoyl, methyl, trifluoromethyl, aminomethyl, 2-(pyrrolidin-1-yl)ethyl, N-methylamino, imidazol-2-ylmethylamino, N-(2-hydroxyethyl)amino, cyclopropylamino, 2-(hydroxymethyl)cyclopropylamino, N-(2-aminoethyl)amino, N-[2-(dimethylamino)ethyl]amino, N-[2-(t-butoxycarbonylamino)ethyl]amino, N,N-dimethylamino, N-methyl-N-(2-hydroxyethyl)amino, N-methyl-N-(2-methoxyethyl)amino, methylthio, N-methylcarbamoyl, N-cyclopropylcarbamoyl, morpholino, 2,6-dimethylmorpholino, 2-(hydroxymethyl)morpholino, piperazin-4-yl, 1-methylpiperazin-4-yl, 1-(t-butoxycarbonyl)piperazin-4-yl, tetrahydropyran-4-ylamino, 2-oxopiperazin-4-yl, 1,4-oxazepan-4-yl, piperidin-1-yl, 3-(hydroxymethyl)piperidin-1-yl, 4-(hydroxymethyl)piperidin-1-yl, 4-hydroxypiperidin-1-yl, 3,4-dihydroxypiperidin-1-yl, piperidin-4-ylamino, 4-cyanoimidazol-5-ylamino, 5-oxo-2,5-dihydro-1H-pyrazol-3-ylamino, pyrazol-4-yl, 3-hydroxypyrrolidin-1-yl, 3,6-dihydropyridin-1(2H)-yl, imidazol-4-yl, pyridin-3-yl, pyridin-4-yl.

X₁ is N; the other X₂, X₃, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; or X₂ and X₅ are N; X₁, X₃ and X₅ are CR¹²; wherein:

R¹² is independently selected from hydrogen, chloro, bromo, cyano, amino, carboxy, carbamoyl, methyl, trifluoromethyl, aminomethyl, 2-(pyrrolidin-1-yl)ethyl, N-methylamino, imidazol-2-ylmethylamino, N-(2-hydroxyethyl)amino, cyclopropylamino, 2-(hydroxymethyl)cyclopropylamino, N-(2-aminoethyl)amino, N-[2-(dimethylamino)ethyl]amino, N-[2-(t-butoxycarbonylamino)ethyl]amino, N,N-dimethylamino, N-methyl-N-(2-hydroxyethyl)amino, N-methyl-N-(2-methoxyethyl)amino, methylthio, N-methylcarbamoyl, N-cyclopropylcarbamoyl, morpholino, 2,6-dimethylmorpholino, 2-(hydroxymethyl)morpholino, piperazin-4-yl, 1-methylpiperazin-4-yl, 1-(t-butoxycarbonyl)piperazin-4-yl, tetrahydropyran-4-ylamino, 2-oxopiperazin-4-yl, 1,4-oxazepan-4-yl, piperidin-1-yl, 3-(hydroxymethyl)piperidin-1-yl, 4-(hydroxymethyl)piperidin-1-yl, 4-hydroxypiperidin-1-yl, 3,4-dihydroxypiperidin-1-yl, piperidin-4-ylamino, 4-cyanoimidazol-5-ylamino, 5-oxo-2,5-dihydro-1H-pyrazol-3-ylamino, pyrazol-4-yl, 3-hydroxypyrrolidin-1-yl, 3,6-dihydropyridin-1(2H)-yl, imidazol-4-yl, pyridin-3-yl, pyridin-4-yl.

X₁ is N; the other X₂, X₃, X₄ and X₅ are CR¹²; or X₂ is N; the other X₁, X₃, X₄ and X₅ are CR¹²; or X₃ is N; the other X₁, X₂, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; wherein

R¹² is independently selected from hydrogen, chloro, trifluoromethyl, methyl, 2-pyrrolidin-1-ylethyl, methylamino, morpholino, 2,6-dimethylmorpholino, piperidin-1-yl, 4-hydroxypiperidin-1-yl, piperazin-1-yl, 3-oxopiperazin-1-yl, 4-methylpiperazin-1-yl, 4-t-butoxycarbonylpiperazin-1-yl, tetrahydropyran-4-ylamino, 1,4-oxazepan-4-yl or N-methyl-N-(2-methoxyethyl)amino.

n is selected from 1 or 2; wherein the values of R¹ may be the same or different.

n is 1.

n is 1; Ring A is phenyl; R¹ is a substituent on carbon and is trifluoromethyl or 1-cyano-1-methylethyl.

n is 1; Ring A is phenyl; R¹ is a substituent on carbon and is 1-cyano-1-methylethyl.

n is 1; Ring A is phenyl; R¹ is a substituent on carbon and is trifluoromethyl or 1-cyano-1-methylethyl and R¹ is meta to the —C(O)NH— group attached to Ring A of formula (I).

n is 1; Ring A is phenyl; R¹ is a substituent on carbon and is 1-cyano-1-methylethyl and R¹ is meta to the —C(O)NH— group attached to Ring A of formula (I).

n is 2; wherein the values of R¹ may be the same or different.

R¹² is independently selected from hydrogen, halo, cyano, amino, carboxy, carbamoyl, C₁₋₆alkyl, N—(C₁₋₆alkyl)amino, N,N—(C₁₋₆alkyl)₂amino, N—(C₁₋₆alkyl)carbamoyl, C₁₋₆alkylS(O)ₐ wherein a is 0, carbocyclyl-R¹⁷— or heterocyclyl-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;

R¹⁹ is selected from halo, cyano, hydroxy, amino, C₁₋₆alkyl, C₁₋₆alkoxy, N,N—(C₁₋₆alkyl)₂amino, C₁₋₆alkoxycarbonylamino or heterocyclyl-R²²—; wherein R¹⁹ may be optionally substituted on carbon by one or more R²³;

R¹⁷, R¹⁸ and R²² are independently selected from a direct bond, —N(R²⁵)— or —N(R²⁶)C(O)—; wherein R²⁵ and R²⁶ are independently selected from hydrogen;

R²⁰ is selected from C₁₋₆alkyl and C₁₋₆alkoxycarbonyl;

R²³ is hydroxy.

R¹² is independently selected from hydrogen, chloro, bromo, cyano, amino, carboxy, carbamoyl, methyl, ethyl, N-methylamino, N-ethylamino, N,N-dimethylamino, N-methyl-N-ethylamino, N-methylcarbamoyl, methylthio, cyclopropyl-R¹⁷—, piperidin-1-yl-R¹⁸—, piperidin-4-yl-R¹⁸—, 1,4-oxazepan-4-yl-R¹⁸—, tetrahydropyran-4-yl-R¹⁸—, piperazin-4-yl-R¹⁸—, imidazol-4-yl-R¹⁸—, imidazol-5-yl-R¹⁸—, 2-oxopiperazin-4-yl-R¹⁸—, 5-oxo-2,5-dihydro-1H-pyrazol-3-yl-R¹⁸—, pyrazol-4-yl-R¹—, pyrrolidin-1-yl-R¹⁵—, pyridin-3-yl-R¹⁸—, pyridin-4-yl-R¹⁸—, 3,6-dihydropyridin-1(2H)-yl-R¹⁸— or morpholino-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;

R¹⁹ is selected from fluoro, cyano, hydroxy, amino, methyl, methoxy, N,N-dimethylamino, t-butoxycarbonylamino, imidazol-2-yl-R²²— or pyrrolidin-1-yl-R²²—; wherein R¹⁹ may be optionally substituted on carbon by one or more R²³;

R¹⁷, R¹⁸ and R²² are independently selected from a direct bond, —N(R²⁵)— or —N(R²⁶)C(O)—; wherein R²⁵ and R²⁶ are independently selected from hydrogen;

R²⁰ is selected from methyl and t-butoxycarbonyl;

R²³ is hydroxy.

R¹² is independently selected from hydrogen, chloro, bromo, cyano, amino, carboxy, carbamoyl, methyl, trifluoromethyl, aminomethyl, 2-(pyrrolidin-1-yl)ethyl, N-methylamino, imidazol-2-ylmethylamino, N-(2-hydroxyethyl)amino, cyclopropylamino, 2-(hydroxymethyl)cyclopropylamino, N-(2-aminoethyl)amino, N-[2-(dimethylamino)ethyl]amino, N-[2-(t-butoxycarbonylamino)ethyl]amino, N,N-dimethylamino, N-methyl-N-(2-hydroxyethyl)amino, N-methyl-N-(2-methoxyethyl)amino, methylthio, N-methylcarbamoyl, N-cyclopropylcarbamoyl, morpholino, 2,6-dimethylmorpholino, 2-(hydroxymethyl)morpholino, piperazin-4-yl, 1-methylpiperazin-4-yl, 1-(t-butoxycarbonyl)piperazin-4-yl, tetrahydropyran-4-ylamino, 2-oxopiperazin-4-yl, 1,4-oxazepan-4-yl, piperidin-1-yl, 3-(hydroxymethyl)piperidin-1-yl, 4-(hydroxymethyl)piperidin-1-yl, 4-hydroxypiperidin-1-yl, 3,4-dihydroxypiperidin-1-yl, piperidin-4-ylamino, 4-cyanoimidazol-5-ylamino, 5-oxo-2,5-dihydro-1H-pyrazol-3-ylamino, pyrazol-4-yl, 3-hydroxypyrrolidin-1-yl, 3,6-dihydropyridin-1(2H)-yl, imidazol-4-yl, pyridin-3-yl, pyridin-4-yl.

Therefore in a further aspect of the invention there is provided a compound of formula (I) (as depicted above) wherein:

Ring A is carbocyclyl;

R¹ is a substituent on carbon and is selected from C₁₋₆alkyl or C₁₋₆alkoxy; wherein R¹ may be optionally substituted on carbon by one or more R⁶;

R² is hydrogen;

X₁ is N; the other X₂, X₃, X₄ and X₅ are CR¹²; or X₁ and X₃ are N; X₂, X₄ and X₅ are CR¹²; or X₂ and X₄ are N; X₂, X₃ and X₅ are CR¹²; or X₂ and X₅ are N; X₂, X₃ and X₄ are CR¹²; or X₂ and A4 are N; X₁, X₃ and X₅ are CR¹²; or X₂ and X₅ are N; X₁, X₃ and X₅ are CR¹²;

R⁶ is selected from halo, cyano or heterocyclyl-R¹⁴—;

R¹² is independently selected from hydrogen, halo, cyano, amino, carboxy, carbamoyl, C₁₋₆alkyl, N—(C₁₋₆alkyl)amino, N)N—(C₁₋₆alkyl)₂amino, N—(C₁₋₆alkyl)carbamoyl, C₁₋₆alkylS(O)ₐ wherein a is 0, carbocyclyl-R¹⁷— or heterocyclyl-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;

R¹⁴ is a direct bond;

R¹⁹ is selected from halo, cyano, hydroxy, amino, C₁₋₆alkyl, C₁₋₆alkoxy, N,N—(C₁₋₆alkyl)₂amino, C₁₋₆alkoxycarbonylamino or heterocyclyl-R²²—; wherein R¹⁹ may be optionally substituted on carbon by one or more R²³;

R¹⁷, R¹⁸ and R²² are independently selected from a direct bond, —N(R²⁵)— or —N(R²⁶)C(O)—; wherein R²⁵ and R²⁶ are independently selected from hydrogen;

R²⁰ is selected from C₁₋₆alkyl and C₁₋₆alkoxycarbonyl;

R²³ is hydroxy;

n is selected from 1 or 2; wherein the values of R¹ may be the same or different;

or a pharmaceutically acceptable salt thereof; with the proviso that said compound is not 4-amino-2-(methylthio)-N-(2-methyl-5-{[3-(trifluoromethyl)benzoyl]amino}phenyl)pyrimidine-5-carboxamide.

Therefore in a further aspect of the invention there is provided the use of a compound of formula (I) (as depicted above) wherein:

Ring A is carbocyclyl;

R¹ is a substituent on carbon and is selected from C₁₋₆alkyl or C₁₋₆alkoxy; wherein R¹ may be optionally substituted on carbon by one or more R⁶;

R² is hydrogen;

X₁ is N; the other X₂, X₃, X₄ and X₅ are CR¹²; or X₂ is N; the other X₁, X₃, X₄ and X₅ are CR¹²; or X₃ is N; the other X₁, X₂, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR²; or X₂ and X₅ are N; X₁, X₃ and X₅ are CR¹²;

R⁶ is selected from halo, cyano or heterocyclyl-R¹⁴—;

R¹² is independently selected from hydrogen, halo, cyano, amino, carboxy, carbamoyl, C₁₋₆alkyl, N—(C₁₋₆alkyl)amino, N,N—(C₁₋₆alkyl)₂amino, N—(C₁₋₆alkyl)carbamoyl, C₁₋₆alkylS(O)ₐ wherein a is 0, carbocyclyl-R¹⁷— or heterocyclyl-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰.

R¹⁴ is a direct bond;

R¹⁹ is selected from halo, cyano, hydroxy, amino, C₁₋₆alkyl, C₁₋₆alkoxy, N,N—(C₁₋₆alkyl)₂amino, C₁₋₆alkoxycarbonylamino or heterocyclyl-R²²—; wherein R¹⁹ may be optionally substituted on carbon by one or more R²³;

R¹⁷, R¹⁸ and R²² are independently selected from a direct bond, —N(R²⁵)— or —N(R²⁶)C(O)—; wherein R²⁵ and R²⁶ are independently selected from hydrogen;

R²⁰ is selected from C₁₋₆alkyl and C₁₋₆alkoxycarbonyl;

R²³ is hydroxy;

n is selected from 1 or 2; wherein the values of R¹ may be the same or different; in the manufacture of a medicament for use in the production of a B-Raf inhibitory effect in a warm-blooded animal such as man.

Therefore in a further aspect of the invention there is provided a compound of formula (I) (as depicted above) wherein:

Ring A is carbocyclyl;

R¹ is a substituent on carbon and is C₁₋₆alkyl; wherein R¹ may be optionally substituted on carbon by one or more R⁶; wherein R⁶ is selected from halo or cyano;

R² is hydrogen;

X₁ is N; the other X₂, X₃, X₄ and X₅ are CR¹²; or X₂ is N; the other X₁, X₃, X₄ and X₅ are CR¹²; or X₃ is N; the other X₁, X₂, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²;

R¹² is independently selected from hydrogen, halo, C₁₋₆alkyl, N—(C₁₋₆alkyl)amino, N,N—(C₁₋₆alkyl)₂amino or heterocyclyl-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;

R¹⁹ is selected from halo, C₁₋₆alkyl, C₁₋₆alkoxy or heterocyclyl-R²²—;

R¹⁸ and R²² are independently selected from a direct bond or —N(R²⁵)—; wherein R²⁵ is selected from hydrogen; and

R²⁰ is selected from C₁₋₆alkyl or C₁₋₆alkoxycarbonyl;

or a pharmaceutically acceptable salt thereof.

Therefore in a further aspect of the invention there is provided a compound of formula (I) (as depicted above) wherein:

Ring A is carbocyclyl;

R¹ is a substituent on carbon and is C₁₋₆alkyl; wherein R¹ may be optionally substituted on carbon by one or more R⁶; wherein R⁶ is selected from halo or cyano;

R² is hydrogen;

X₁ is N; the other X₂, X₃, X₄ and X₅ are CR¹²; or X₂ is N; the other X₁, X₃, X₄ and X₅ are CR¹²; or X₃ is N; the other X₁, X₂, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²;

n is selected from 0-4; wherein the values of R¹ may be the same or different;

R¹² is independently selected from hydrogen, halo, C₁₋₆alkyl, N—(C₁₋₆alkyl)amino, N,N—(C₁₋₆alkyl)₂amino or heterocyclyl-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;

R¹⁹ is selected from halo, C₁₋₆alkyl, C₁₋₆alkoxy or heterocyclyl-R²²—;

R¹⁸ and R²² are independently selected from a direct bond or —N(R²⁵)—; wherein R¹⁵ is selected from hydrogen; and

R²⁰ is selected from C₁₋₆alkyl or C₁₋₆alkoxycarbonyl;

or a pharmaceutically acceptable salt thereof.

Therefore in a further aspect of the invention there is provided a compound of formula (I) (as depicted above) wherein:

Ring A is phenyl;

R¹ is a substituent on carbon and is trifluoromethyl, 1-cyano-1-methylethyl or 2-(morpholino)ethoxy;

R² is hydrogen;

X₁ is N; the other X₂, X₃, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; or X₂ and X₅ are N; X₁, X₃ and X₅ are CR¹²;

R¹² is independently selected from hydrogen, chloro, bromo, cyano, amino, carboxy, carbamoyl, methyl, trifluoromethyl, aminomethyl, 2-(pyrrolidin-1-yl)ethyl, N-methylamino, imidazol-2-ylmethylamino, N-(2-hydroxyethyl)amino, cyclopropylamino, 2-(hydroxymethyl)cyclopropylamino, N-(2-aminoethyl)amino, N-[2-(dimethylamino)ethyl]amino, N-[2-(t-butoxycarbonylamino)ethyl]amino, N,N-dimethylamino, N-methyl-N-(2-hydroxyethyl)amino, N-methyl-N-(2-methoxyethyl)amino, methylthio, N-methylcarbamoyl, N-cyclopropylcarbamoyl, morpholino, 2,6-dimethylmorpholino, 2-(hydroxymethyl)morpholino, piperazin-4-yl, 1-methylpiperazin-4-yl, 1-(t-butoxycarbonyl)piperazin-4-yl, tetrahydropyran-4-ylamino, 2-oxopiperazin-4-yl, 1,4-oxazepan-4-yl, piperidin-1-yl, 3-(hydroxymethyl)piperidin-1-yl, 4-(hydroxymethyl)piperidin-1-yl, 4-hydroxypiperidin-1-yl, 3,4-dihydroxypiperidin-1-yl, piperidin-4-ylamino, 4-cyanoimidazol-5-ylamino, 5-oxo-2,5-dihydro-1H-pyrazol-3-ylamino, pyrazol-4-yl, 3-hydroxypyrrolidin-1-yl, 3,6-dihydropyridin-1(2H)-yl, imidazol-4-yl, pyridin-3-yl, pyridin-4-yl;

n is selected from 1 or 2; wherein the values of R¹ may be the same or different;

or a pharmaceutically acceptable salt thereof with the proviso that said compound is not 4-amino-2-(methylthio)-N-(2-methyl-5-{[3-(trifluoromethyl)benzoyl]amino}phenyl)pyrimidine-5-carboxamide.

Therefore in a farther aspect of the invention there is provided the use of a compound of formula (I) (as depicted above) wherein:

Ring A is phenyl;

R¹ is a substituent on carbon and is trifluoromethyl, 1-cyano-1-methylethyl or 2-(morpholino)ethoxy;

R² is hydrogen;

X₁ is N; the other X₂, X₃, X₄ and X₅ are CR¹²; or X₂ is N; the other X₁, X₃, X₄ and X₅ are CR¹²; or X₃ is N; the other X₁, X₂, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; or X₂ and X₅ are N; X₁, X₃ and X₅ are CR¹²;

R¹² is independently selected from hydrogen, chloro, bromo, cyano, amino, carboxy, carbamoyl, methyl, trifluoromethyl, aminomethyl, 2-(pyrrolidin-1-yl)ethyl, N-methylamino, imidazol-2-ylmethylamino, N-(2-hydroxyethyl)amino, cyclopropylamino, 2-(hydroxymethyl)cyclopropylamino, N-(2-aminoethyl)amino, N-[2-(dimethylamino)ethyl]amino, N-[2-(t-butoxycarbonylamino)ethyl]amino, N,N-dimethylamino, N-methyl-N-(2-hydroxyethyl)amino, N-methyl-N-(2-methoxyethyl)amino, methylthio, N-methylcarbamoyl, N-cyclopropylcarbamoyl, morpholino, 2,6-dimethylmorpholino, 2-(hydroxymethyl)morpholino, piperazin-4-yl, 1-methylpiperazin-4-yl, 1-(t-butoxycarbonyl)piperazin-4-yl, tetrahydropyran-4-ylamino, 2-oxopiperazin-4-yl, 1,4-oxazepan-4-yl, piperidin-1-yl, 3-(hydroxymethyl)piperidin-1-yl, 4-(hydroxymethyl)piperidin-1-yl, 4-hydroxypiperidin-1-yl, 3,4-dihydroxypiperidin-1-yl, piperidin-4-ylamino, 4-cyanoimidazol-5-ylamino, 5-oxo-2,5-dihydro-1H-pyrazol-3-ylamino, pyrazol-4-yl, 3-hydroxypyrrolidin-1-yl, 3,6-dihydropyridin-1(2H)-yl, imidazol-4-yl, pyridin-3-yl, pyridin-4-yl;

n is selected from 1 or 2; wherein the values of R¹ may be the same or different;

or a pharmaceutically acceptable salt thereof in the manufacture of a medicament for use in the production of a B-Raf inhibitory effect in a warm-blooded animal such as man.

Therefore in a further aspect of the invention there is provided a compound of formula (I) (as depicted above) wherein:

Ring A is phenyl;

R¹ is a substituent on carbon and is trifluoromethyl or 1-cyano-1-methylethyl;

R² is hydrogen;

X₁ is N; the other X₂, X₃, X₄ and X₅ are CR¹²; or X₂ is N; the other X₁, X₃, X₄ and X₅ are CR¹²; or X₃ is N; the other X₁, X₂, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; and

R¹² is independently selected from hydrogen, chloro, trifluoromethyl, methyl, 2-pyrrolidin-1-ylethyl, methylamino, morpholino, 2,6-dimethylmorpholino, piperidin-1-yl, 4-hydroxypiperidin-1-yl, piperazin-1-yl, 3-oxopiperazin-1-yl, 4-methylpiperazin-1-yl, 4-t-butoxycarbonylpiperazin-1-yl, tetrahydropyran-4-ylamino, 1,4-oxazepan-4-yl or N-methyl-N-(2-methoxyethyl)amino;

or a pharmaceutically acceptable salt thereof.

Therefore in a further aspect of the invention there is provided a compound of formula (I) (as depicted above) wherein:

Ring A is phenyl;

R¹ is a substituent on carbon and is trifluoromethyl or 1-cyano-1-methylethyl;

R¹ is hydrogen;

X₁ is N; the other X₂, X₃, X₄ and X₅ are CR²; or X₂ is N; the other X₁, X₃, X₄ and X₅ are CR¹²; or X₃ is N; the other X₁, X₂, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; and

n is selected from 0-4; wherein the values of R¹ may be the same or different;

R¹² is independently selected from hydrogen, chloro, trifluoromethyl, methyl, 2-pyrrolidin-1-ylethyl, methylamino, morpholino, 2,6-dimethylmorpholino, piperidin-1-yl, 4-hydroxypiperidin-1-yl, piperazin-1-yl, 3-oxopiperazin-1-yl, 4-methylpiperazin-1-yl, 4-t-butoxycarbonylpiperazin-1-yl, tetrahydropyran-4-ylamino, 1,4-oxazepan-4-yl or N-methyl-N-(2-methoxyethyl)amino;

or a pharmaceutically acceptable salt thereof.

In another aspect of the invention, preferred compounds of the invention are any one of the Examples or a pharmaceutically acceptable salt thereof.

In another aspect of the invention, particular compounds of the invention are Examples 7, 11, 30, 31, 35, 36, 47, 59 and 73 or a pharmaceutically acceptable salt thereof.

Another aspect of the present invention provides a process for preparing a compound of formula (I) or a pharmaceutically acceptable salt thereof which process (wherein variable are, unless otherwise specified, as defined in formula (I)) comprises of:
Process a) reacting an amine of the formula (II) 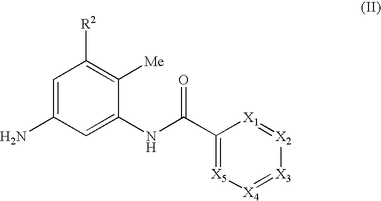
with an acid of formula (III): 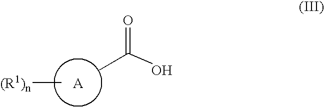
or an activated acid derivative thereof;
Process b) reacting an amine of formula (VI): 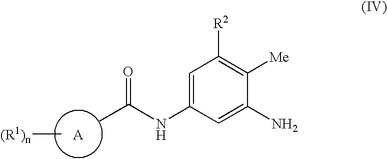
with an acid of formula (V): 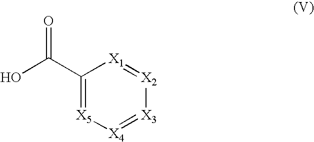
or an activated acid derivative thereof;
and thereafter if necessary:
i) converting a compound of the formula (I) into another compound of the formula (I);
ii) removing any protecting groups;
iii) forming a pharmaceutically acceptable salt.

Specific reaction conditions for the above reactions are as follows.

Process a) and Process b) Amines and acids may be coupled together in the presence of a suitable coupling reagent. Standard peptide coupling reagents known in the art can be employed as suitable coupling reagents, or for Example carbonyldimidazole and dicyclohexyl-carbodiimide, optionally in the presence of a catalyst such as dimethylaminopyridine or 4-pyrrolidinopyridine, optionally in the presence of a base for Example triethylamine, pyridine, or 2,6-di-alkyl-pyridines such as 2,6-lutidine or 2,6-di-tert-butylpyridine. Suitable solvents include dimethylacetamide, dichloromethane, benzene, tetrahydrofuran and dimethylformamide. The coupling reaction may conveniently be performed at a temperature in the range of −40 to 40° C.

Suitable activated acid derivatives include acid halides, for Example acid chlorides, and active esters, for Example pentafluorophenyl esters. The reaction of these types of compounds with amines is well known in the art, for Example they may be reacted in the presence of a base, such as those described above, and in a suitable solvent, such as those described above. The reaction may conveniently be performed at a temperature in the range of −40 to 40° C.

Amines of formula (II) may be prepared according to Scheme 1:

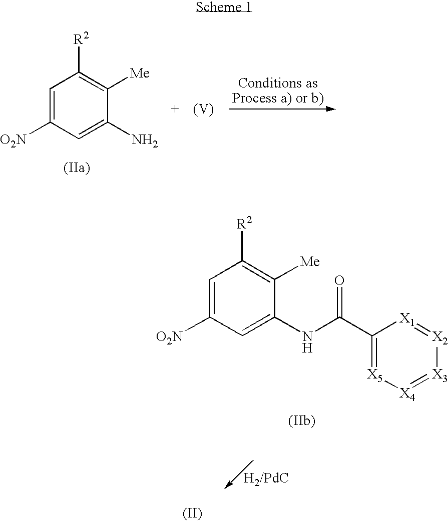

Amines of formula (IV) may be prepared according to Scheme 2:

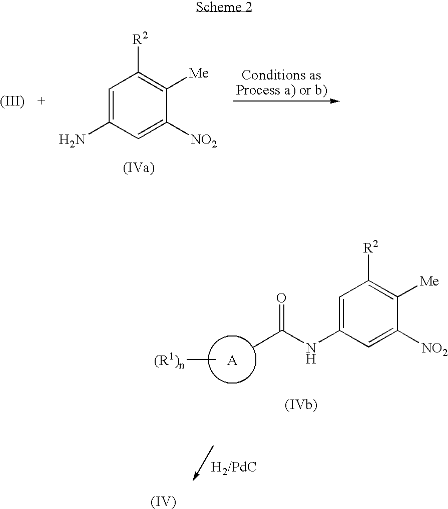

Compounds of formula (III), (V), (IIa) and (IVa) are commercially available compounds, or they are known in the literature or they may be prepared by standard processes known in the art.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possesses anti-cancer activity which is believed to arise from the B-Raf inhibitory activity of the compound. These properties may be assessed, for example, using the procedure set out below.

B-Raf In Vitro ELISA Assay

Activity of human recombinant, purified wild type His-B-Raf protein kinase was determined in vitro using an enzyme-linked immunosorbent assay (ELISA) assay format, which measures phosphorylation of the B-Raf substrate, human recombinant, purified His-derived (detagged) MEK1. The reaction utilized 2.5 nM B-Raf, 0.15 μM MEK1 and 10 μM adenosine triphosphate (ATP) in 40 mM N-(2-hydroxyethyl)piperazine-N′-(2-ethanesulfonic acid hemisodium salt (HEPES), 5 mM 1,4-dithio-DL-threitol (DTT), 10 mM MgCl₂, 1 mM ethylenediaminetetraacetic acid (EDTA) and 0.2 M NaCl (1×HEPES buffer), with or without compound at various concentrations, in a total reaction volume of 25 μl in 384 well plates. B-Raf and compound were preincubated in 1×HEPES buffer for 1 hour at 25° C. Reactions were initiated with addition of MEK1 and ATP in 1×HEPES buffer and incubated at 25° C. for 50 minutes and reactions stopped by addition of 10 μl 175 mM EDTA (final concentration 50 mM) in 1× HEPES buffer. 5 μl of the assay mix was then diluted 1:20 into 50 mM EDTA in 1×HEPES buffer, transferred to 384 well black high protein binding plates and incubated overnight at 4° C. Plates were washed in tris buffered saline containing 0.1% Tween20 (TBST), blocked with 50 μl Superblock (Pierce) for 1 hour at 25° C., washed in TBST, incubated with 50 μl rabbit polyclonal anti-phospho-MEK antibody (Cell Signaling) diluted 1:1000 in TBS for 2 hours at 25° C., washed with TBST, incubated with 50 μl goat anti-rabbit horseradish peroxidase-linked antibody (Cell Signaling) diluted 1:2000 in TBS for 1 hour at 25° C. and washed with TBST. 50 μl of fluorogenic peroxidase substrate (Quantablu—Pierce) was added and following incubation for 45-60 minutes, 50 μl QuantabluSTOP (Pierce) was added. Blue fluorescent product was detected at excitation 325 and emission 420 using a TECAN Ultra plate reader. Data was graphed and IC₅₀s calculated using Excel Fit (Microsoft).

When tested in the above in vitro assay, the compounds of the present invention exhibited activity less than 30 μM. For example the following results were obtained:

| Example No | IC₅₀ (μM) |
|---|---|
| 1 | 5.7 |
| 7 | 0.6 |
| 49 | 0.8 |

According to a further aspect of the invention there is provided a pharmaceutical composition which comprises a compound of the formula (I), or a pharmaceutically acceptable salt thereof, as defined hereinbefore, in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I) will normally be administered to a warm-blooded animal at a unit dose within the range 1-1000 mg/kg, and this normally provides a therapeutically-effective dose. Preferably a daily dose in the range of 10-100 mg/kg is employed. However the daily dose will necessarily be varied depending upon the host treated, the particular route of administration, and the severity of the illness being treated. Accordingly the optimum dosage may be determined by the practitioner who is treating any particular patient.

According to a further aspect of the present invention there is provided a compound of the formula (I), or a pharmaceutically acceptable salt thereof, as defined hereinbefore for use in a method of treatment of the human or animal body by therapy.

We have found that the compounds defined in the present invention, or a pharmaceutically acceptable salt thereof, are effective anti-cancer agents which property is believed to arise from their B-Raf inhibitory properties. Accordingly the compounds of the present invention are expected to be useful in the treatment of diseases or medical conditions mediated alone or in part by B-Raf, i.e. the compounds may be used to produce a B-Raf inhibitory effect in a warm-blooded animal in need of such treatment.

Thus the compounds of the present invention provide a method for treating cancer characterised by inhibition of B-Raf, i.e. the compounds may be used to produce an anti-cancer effect mediated alone or in part by the inhibition of B-Raf.

Such a compound of the invention is expected to possess a wide range of anti-cancer properties as activating mutations in B-Raf have been observed in many human cancers, including but not limited to, melanoma, papillary thyroid tumors, cholangiocarcinomas, colon, ovarian and lung cancers. Thus it is expected that a compound of the invention will possess anti-cancer activity against these cancers. It is in addition expected that a compound of the present invention will possess activity against a range of leukaemias, lymphoid malignancies and solid tumours such as carcinomas and sarcomas in tissues such as the liver, kidney, bladder, prostate, breast and pancreas. In particular such compounds of the invention are expected to slow advantageously the growth of primary and recurrent solid tumours of, for example, the skin, colon, thyroid, lungs and ovaries. More particularly such compounds of the invention, or a pharmaceutically acceptable salt thereof, are expected to inhibit the growth of those primary and recurrent solid tumours which are associated with B-Raf, especially those tumours which are significantly dependent on B-Raf for their growth and spread, including for example, certain tumours of the skin, colon, thyroid, lungs and ovaries. Particularly the compounds of the present invention are useful in the treatment of melanomas.

Herein where producing an “an anti-cancer effect” is referred to, this term includes both the prophylaxis and the treatment of cancer. Prophylaxis and treatment of cancer includes the prophylaxis and treatment of the primary tumour, secondary tumours and any metastases.

Thus according to this aspect of the invention there is provided a compound of the formula (I), or a pharmaceutically acceptable salt thereof, as defined hereinbefore for use as a medicament.

According to a further aspect of the invention there is provided the use of a compound of the formula (a), or a pharmaceutically acceptable salt thereof, as defined hereinbefore in the manufacture of a medicament for use in the production of a B-Raf inhibitory effect in a warm-blooded animal such as man.

According to this aspect of the invention there is provided the use of a compound of the formula (I), or a pharmaceutically acceptable salt thereof, as defined hereinbefore in the manufacture of a medicament for use in the production of an anti-cancer effect in a warm-blooded animal such as man.

According to a further feature of the invention, there is provided the use of a compound of the formula (I), or a pharmaceutically acceptable salt thereof, as defined herein before in the manufacture of a medicament for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further aspect of the invention there is provided the use of a compound of the formula (I), or a pharmaceutically acceptable salt thereof, as defined hereinbefore for the production of a B-Raf inhibitory effect in a warm-blooded animal such as man.

According to this aspect of the invention there is provided the use of a compound of the formula (I), or a pharmaceutically acceptable salt thereof, as defined hereinbefore for the production of an anti-cancer effect in a warm-blooded animal such as man.

According to a further feature of the invention, there is provided the use of a compound of the formula (I), or a pharmaceutically acceptable salt thereof, as defined herein before for the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries.

According to a further feature of this aspect of the invention there is provided a method for producing a B-Raf inhibitory effect in a warm-blooded animal, such as man, in need of such treatment which comprises administering to said animal an effective amount of a compound of formula (I), or a pharmaceutically acceptable salt thereof, as defined above.

According to a further feature of this aspect of the invention there is provided a method for producing an anti-cancer effect in a warm-blooded animal, such as man, in need of such treatment which comprises administering to said animal an effective amount of a compound of formula (I), or a pharmaceutically acceptable salt thereof, as defined above.

According to an additional feature of this aspect of the invention there is provided a method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I) or a pharmaceutically acceptable salt thereof as defined herein before.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of a B-Raf inhibitory effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the production of an anti-cancer effect in a warm-blooded animal such as man.

In a further aspect of the invention there is provided a pharmaceutical composition which comprises a compound of the formula (I), or a pharmaceutically acceptable salt thereof, as defined herein before in association with a pharmaceutically-acceptable diluent or carrier for use in the treatment of melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries in a warm-blooded animal such as man.

For the avoidance of doubt, where the use of a compound of formula (I) is referred to in a method of treatment, in the manufacture of a medicament, in the production of a B-Raf inhibitory effect, in the production of an anti-cancer effect or the treatment of certain specified cancers, it is to be understood that this refers to any definition of the compound of formula (I) given herein.

In a further aspect of the invention where the use of a compound of formula (I) is referred to, for example as a medicament, in a method of treatment, in the manufacture of a medicament, in a pharmaceutical composition, in the production of a B-Raf inhibitory effect, in the production of an anti-cancer effect or the treatment of certain specified cancers the compound of formula (I) includes 4-amino-2-(methylthio)-N-(2-methyl-5-{[3-(trifluoromethyl)benzoyl]amino}phenyl)pyrimidine-5-carboxamide.

The B-Raf inhibitory treatment defined hereinbefore may be applied as a sole therapy or may involve, in addition to the compound of the invention, conventional surgery or radiotherapy or chemotherapy. Such chemotherapy may include one or more of the following categories of anti-tumour agents:—

(i) antiproliferative/antineoplastic drugs and combinations thereof, as used in medical oncology, such as alkylating agents (for example cis-platin, carboplatin, cyclophosphamide, nitrogen mustard, melphalan, chlorambucil, busulphan and nitrosoureas); antimetabolites (for example antifolates such as fluoropyrimidines like 5-fluorouracil and tegaflir, raltitrexed, methotrexate, cytosine arabinoside and hydroxyurea; antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere); and topoisomerase inhibitors (for example epipodophyllotoxins like etoposide and teniposide, amsacrine, topotecan and camptothecin);

(ii) cytostatic agents such as antioestrogens (for example tamoxifen, toremifene, raloxifene, droloxifene and iodoxyfene), oestrogen receptor down regulators (for example fulvestrant), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5α-reductase such as finasteride;

(iii) Agents which inhibit cancer cell invasion (for example metalloproteinase inhibitors like marimastat and inhibitors of urokinase plasminogen activator receptor function);

(iv) inhibitors of growth factor function, for example such inhibitors include growth factor antibodies, growth factor receptor antibodies (for example the anti-erbb2 antibody trastuzumab [Herceptin™] and the anti-erbb1 antibody cetuximab [C225]), farnesyl transferase inhibitors, MEK inhibitors, tyrosine kinase inhibitors and serine/threonine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, AZD 1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)quinazolin-4-amine (CI 1033)), for example inhibitors of the platelet-derived growth factor family and for example inhibitors of the hepatocyte growth factor family;

(v) antiangiogenic agents such as those which inhibit the effects of vascular endothelial growth factor, (for example the anti-vascular endothelial cell growth factor antibody bevacizumab [Avastin™], compounds such as those disclosed in International Patent Applications WO 97/22596, WO 97/30035, WO 97/32856 and WO 98/13354) and compounds that work by other mechanisms (for example linomide, inhibitors of integrin αᵥβ₃ function and angiostatin);

(vi) vascular damaging agents such as Combretastatin A4 and compounds disclosed in International Patent Applications WO 99/02166, WO00/40529, WO 00/41669, WO01/92224, WO02/04434 and WO02/08213;

(vii) antisense therapies, for example those which are directed to the targets listed above, such as ISIS 2503, an anti-ras antisense;

(viii) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy;

(ix) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies;

(x) Cell cycle inhibitors including for example CDK inhibitors (eg flavopiridol) and other inhibitors of cell cycle checkpoints (eg checkpoint kinase); inhibitors of aurora kinase and other kinases involved in mitosis and cytokinesis regulation (eg mitotic kinesins); and histone deacetylase inhibitors; and

(xi) endothelin antagonists, including endothelin A antagonists, enddthelin B antagonists and endothelin A and B antagonists; for example ZD4054 and ZD1611 (WO 96 40681), atrasentan and YM598.

Such conjoint treatment may be achieved by way of the simultaneous, sequential or separate dosing of the individual components of the treatment. Such combination products employ the compounds of this invention within the dosage range described hereinbefore and the other pharmaceutically-active agent within its approved dosage range.

In addition to their use in therapeutic medicine, the compounds of formula (I) and their pharmaceutically acceptable salts are also useful as pharmacological tools in the development and standardisation of in vitro and in vivo test systems for the evaluation of the effects of inhibitors of B-Raf in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

### EXAMPLES

The invention will now be illustrated by the following non limiting examples in which, unless stated otherwise:

(i) temperatures are given in degrees Celsius (° C.); operations were carried out at room or ambient temperature, that is, at a temperature in the range of 18-25° C.;

(ii) organic solutions were dried over anhydrous sodium sulphate; evaporation of solvent was carried out using a rotary evaporator under reduced pressure (600-4000 Pascals; 4.5-30 mmHg) with a bath temperature of up to 60° C.;

(iii) in general, the course of reactions was followed by TLC and reaction times are given for illustration only;

(iv) final products had satisfactory proton nuclear magnetic resonance (NMR) spectra and/or mass spectral data;

(v) yields are given for illustration only and are not necessarily those which can be obtained by diligent process development; preparations were repeated if more material was required;

(vii) when given, NMR data is in the form of delta values for major diagnostic protons, given in parts per million (ppm) relative to tetramethylsilane (TMS) as an internal standard, determined at 400 MHz using perdeuterio dimethyl sulphoxide (DMSO-d₆) as solvent unless otherwise indicated;

(vii) chemical symbols have their usual meanings; SI units and symbols are used;

(viii) solvent ratios are given in volume:volume (v/v) terms; and

(ix) mass spectra were run with an electron energy of 70 electron volts in the chemical ionization (CI) mode using a direct exposure probe; where indicated ionization was effected by electron impact (EI), fast atom bombardment (FAB) or electrospray (ESP); values for m/z are given; generally, only ions which indicate the parent mass are reported; and unless otherwise stated, the mass ion quoted is (MH)⁺;

(x) where a synthesis is described as being analogous to that described in a previous example the amounts used are the millimolar ratio equivalents to those used in the previous example;

(xi) the following abbreviations have been used:

THF tetrahydrofuran;
DMF N,N-dimethylformamide;
EtOAc ethyl acetate;
EDCI 1-(3-dimethylaminopropyl)-3-ethylcarbodiimide hydrochloride;
HOBt Hydroxybenzotriazole;
HATU O-(7-azabenzotriazol-1-yl)-N,N,N′N′-tetramethyluronium hexafluorophosphate;
DIEA diisopropylethylamine;
mCPBA 3-chloroperoxybenzoic acid;
SM starting material;
DCM dichloromethane; and
DMSO dimethylsulphoxide;
   (xii) “ISCO” refers to normal phase flash column chromatography using 12 g and 40 g pre-packed silica gel cartridges used according to the manufacturers instruction obtained from ISCO, Inc, 4700 Superior Street Lincoln, Nebr., USA; and
   (xiii) “SmithSynthesizer” refers to a microwave produced by Personal Chemistry (Now Biotage) and used according to the manufacturers instruction obtained from Biotage, 1725 Discovery Drive, Charlottesville, Va. 22911, USA.

### Example 1

### N¹-[3-(1-Cyano-1-methylethyl)benzoyl]-N³-[2-(piperidin-1-yl)-4-methylpyrimidin-6-ylcarbonyl]-4-methylbenzene-1,3-diamine

N-(3-Amino-4-methylphenyl)-3-(1-cyano-1-methylethyl)benzamide (Method 59; 0.100 g, 0.34 mmol), 6-methyl-2-piperidin-1-ylpyrimidine-4-carboxylic acid (Method 19; 0.075 g, 0.34 mmol), HATU (0.14 g, 0.037 mmol) and DIEA (0.18 ml, 1.02 mmol) were combined in 8 ml anhydrous DMF and the reaction mixture was allowed to stir at 25° C. for 15 hours. The reaction mixture was concentrated under reduced pressure and purified by reverse phase semi-preparative chromatography. NMR (400 MHz): 10.30 (s, 1H), 10.09 (s, 1H), 8.17-8.22 (m, 1H), 7.99 (s, 1H), 7.89 (d, 1H), 7.68 (d, 1H), 7.49-7.58 (m, 2H), 7.20 (d, 1H), 7.03 (s, 1H), 3.73-3.85 (m, 4H), 2.34 (s, 3H), 2.21 (s, 3H), 1.69 (s, 6H), 1.54-1.63 (m, 2H), 1.43-1.55 (m, 4H), m/z 499.

### Examples 2-47

The following compounds were prepared by the procedure of Example 1, using the appropriate carboxylic acid (commercially available unless otherwise indicated) and N-(3-amino-4-methylphenyl)-3-(1-cyano-1-methylethyl)benzamide (Method 59) or N-(3-amino-4-methylphenyl)-3-(1-cyano-1-methylethyl)-5-(2-morpholin-4-ylethoxy)benzamide (Method 60) as starting materials.

| Ex | Compound | NMR | m/z | SM |
|---|---|---|---|---|
| 2 | N¹-[3-(1-Cyano-1- | 10.31(s, 1H), 10.07(s, 1H), 8.29(d, 1H), | 513 | Method |
| | methylethyl)benzoyl]-N³- | 8.00(s, 1H), 7.89(d, 1H), 7.68(d, | | 23 |
| | [2-(1,4-oxazepan-4-yl)-4- | 1H), 7.45-7.60(m, 2H), 7.20(d, 1H), | | |
| | methylpyrimidin-6- | 7.05-7.13(m, 1H), 3.78-4.01(m, 4H), | | |
| | ylcarbonyl]-4-methyl | 3.64-3.78(m, 2H), 3.58(t, 2H), | | |
| | benzene-1,3-diamine | 2.36(s, 3H), 2.23(s, 3H), | | |
| | | 1.77-1.93(m, 2H), 1.69(s, 6H) | | |
| 3 | N¹-[3-(1-Cyano-1- | 10.28(s, 1H), 10.13(s, 1H), 8.55(s, 1H), | 519 | Method |
| | methylethyl)benzoyl]-N³- | 7.99(s, 1H), 7.85-7.92(m, 2H), | | 32 |
| | [2-(morpholino)-5-chloro | 7.68(d, 1H), 7.58(dd, 1H), 7.53(t, 1H), | | |
| | pyrimidin-6-ylcarbonyl]- | 7.20(d, 1H), 3.66-3.73(m, 4H), | | |
| | 4-methyl benzene-1,3- | 3.56-3.66(m, 4H), 2.19(s, 3H), | | |
| | diamine | 1.69(s, 6H) | | |
| 4 | N¹-[3-(1-Cyano-1- | 10.33(s, 1H), 7.98(s, 1H), 7.87(d, 1H), | 514 | Method |
| | methylethyl)benzoyl]-N³- | 7.69(d, 1H), 7.44-7.59(m, 2H), | | 68 |
| | [2-(methylamino)-4- | 7.17-7.27(m, 2H), 7.07(s, 1H), | | |
| | (morpholino)pyrimidin-6- | 6.90(s, 1H), 3.49-3.77(m, 8H), 2.84(s, 3H), | | |
| | ylcarbonyl]-4-methyl | 2.18(s, 3H), 1.69(s, 6H) | | |
| | benzene-1,3-diamine | | | |
| 5 | N¹-[3-(1-Cyano-1- | 10.31(s, 1H), 10.20(s, 1H), 8.02(d, 1H), | 512 | Method |
| | methylethyl)benzoyl]-N³- | 7.98(s, 1H), 7.88(d, 1H), 7.69(d, | | 21 |
| | [2-(4-methylpiperazin-1- | 1H), 7.49-7.58(m, 2H), | | |
| | yl)-4-methylpyrimidin-6- | 7.17-7.27(m, 2H), 4.83-4.97(m, 2H), | | |
| | ylcarbonyl]-4-methyl | 3.41-3.51(m, 2H), 3.17-3.31(m, 2H), | | |
| | benzene-1,3-diamine | 2.95-3.09(m, 2H), 2.79(s, 3H), 2.40(s, 3H), | | |
| | | 2.18(s, 3H), 1.69(s, 6H) | | |
| 6 | N¹-[3-(1-Cyano-1- | 10.30(s, 1H), 10.05(s, 1H), 8.41(s, 1H), | 513 | Method |
| | methylethyl)benzoyl]-N³- | 7.99(s, 1H), 7.89(d, 1H), 7.68(d, | | 20 |
| | [2-(tetrahydro-2H-pyran-4- | 1H), 7.48-7.57(m, 2H), 7.20(d, 1H), | | |
| | ylamino)-4-methyl | 7.06(s, 1H), 4.03(s, 1H), | | |
| | pyrimidin-6-ylcarbonyl]-4- | 3.77-3.89(m, 2H), 3.26-3.42(m, 2H), 2.32(s, 3H), | | |
| | methylbenzene-1,3- | 2.27(s, 3H), 1.78-1.89(m, 2H), | | |
| | diamine | 1.69(s, 6H), 1.41-1.55(m, 2H) | | |
| 7 | N¹-[3-(1-Cyano-1- | 10.30(s, 1H), 10.13(s, 1H), 8.10(d, 1H), | 499 | Method |
| | methylethyl)benzoyl]-N³- | 7.95-8.01(m, 1H), 7.89(d, 1H), | | 22 |
| | [2-(morpholino)-4-methyl | 7.63-7.73(m, 1H), 7.55(s, 2H), | | |
| | pyrimidin-6-ylcarbonyl]-4- | 7.21(d, 1H), 7.12(s, 1H), 3.77(s, 4H), | | |
| | methylbenzene-1,3- | 3.63(s, 4H), 2.36(s, 3H), 2.20(s, 3H), | | |
| | diamine | 1.69(s, 6H) | | |
| 8 | N¹-[3-(1-Cyano-1- | 10.25(s, 1H), 9.96(s, 1H), 7.98(s, 1H), | 498 | Method |
| | methylethyl)benzoyl]-N³- | 7.87(d, 1H), 7.73(s, 1H), 7.68(d, | | 31 |
| | [2-methyl-6-(morpholino) | 1H), 7.49-7.56(m, 2H), 7.20(d, 1H), | | |
| | pyridin-4-ylcarbonyl]-4- | 7.07(s, 1H), 6.98(s, 1H), | | |
| | methylbenzene-1,3- | 3.62-3.70(m, 4H), 3.43-3.50(m, 4H), 2.35(s, 3H), | | |
| | diamine | 2.13(s, 3H), 1.68(s, 6H) | | |
| 9 | N¹-[3-(1-Cyano-1- | 10.28(s, 1H), 10.07(s, 1H), 8.21(d, 1H), | 484 | |
| | methylethyl)benzoyl]-N³- | 7.98(s, 1H), 7.87(d, 1H), | | |
| | [6-(morpholino)pyridin-4- | 7.71-7.80(m, 1H), 7.63-7.72(m, 1H), | | |
| | ylcarbonyl]-4-methyl | 7.46-7.59(m, 2H), 7.34(s, 1H), 7.21(d, 1H), | | |
| | benzene-1,3-diamine | 7.12(d, 1H), 3.60-3.74(m, 4H), | | |
| | | 3.44-3.56(m, 4H), 2.14(s, 3H), | | |
| | | 1.68(s, 6H) | | |
| 10 | N¹-[3-(1-Cyano-1- | 10.29(s, 1H), 9.98(s, 1H), 8.07(d, 1H), | 519 | Method |
| | methylethyl)benzoyl]-N³- | 7.94-8.00(m, 1H), 7.87(d, 1H), | | 29 |
| | [2-chloro-4-(morpholino) | 7.63-7.74(m, 1H), 7.46-7.58(m, 2H), | | |
| | pyrimidin-6-ylcarbonyl]-4- | 7.32(s, 1H), 7.20(d, 1H), | | |
| | methylbenzene-1,3- | 3.54-3.75(m, 8H), 2.17(s, 3H), 1.69(s, 6H) | | |
| | diamine | | | |
| 11 | N¹-[3-(1-Cyano-1- | 10.30(s, 1H), 10.21(s, 1H), | 512 | Method |
| | methylethyl)benzoyl]-N³- | 8.04-8.11(m, 2H), 7.96-8.02(m, 1H), | | 25 |
| | [2-(3-oxopiperazin-1-yl)- | 7.89(d, 1H), 7.65-7.72(m, 1H), | | |
| | 4-methylpyrimidin-6- | 7.48-7.60(m, 2H), 7.21(d, 1H), 7.16(s, 1H), | | |
| | ylcarbonyl]-4-methyl | 4.23-4.32(m, 2H), 3.92-4.01(m, | | |
| | benzene-1,3-diamine | 2H), 3.16-3.30(m, 2H), 2.38(s, 3H), | | |
| | | 2.21(s, 3H), 1.69(s, 6H) | | |
| 12 | N¹-[3-(1-Cyano-1- | 10.31(s, 2H), 10.07(s, 1H), | 501 | Method |
| | methylethyl)benzoyl]-N³- | 8.30-8.38(m, 1H), 8.00(s, 1H), 7.89(d, 3H), | | 24 |
| | {2-[N-methyl-N-(2- | 7.68(d, 1H), 7.49-7.60(m, 2H), | | |
| | methoxyethyl)amino]-4- | 7.20(d, 1H), 7.07(s, 1H), | | |
| | methylpyrimidin-6- | 3.75-3.87(m, 2H), 3.44-3.59(m, 2H), 3.21(s, 3H), | | |
| | ylcarbonyl}-4-methyl | 3.16(s, 3H), 2.35(s, 3H), 2.24(s, 3H), | | |
| | benzene-1,3-diamine | 1.69(s, 6H) | | |
| 13 | N¹-[3-(1-Cyano-1- | 10.30(s, 1H), 10.14(s, 1H), 7.99(s, 1H), | 527 | Method |
| | methylethyl)benzoyl]-N³- | 7.89(d, 1H), 7.63-7.73(m, 1H), | | 28 |
| | [2-(2,6-dimethyl | 7.46-7.60(m, 2H), 7.21(d, 1H), | | |
| | morpholino)-4-methyl | 7.10(s, 1H), 4.53-4.75(m, 2H), | | |
| | pyrimidin-6-ylcarbonyl]-4- | 3.47-3.61(m, 2H), 2.46-2.58(m, 2H), | | |
| | methylbenzene-1,3- | 2.36(s, 3H), 2.21(s, 3H), 1.69(s, 6H) | | |
| | diamine | | | |
| 14 | N¹-[3-(1-Cyano-1- | 10.28(s, 1H), 10.07(s, 1H), | 518 | Method |
| | methylethyl)benzoyl]-N³- | 7.94-8.00(m, 1H), 7.87(d, 1H), | | 30 |
| | [2-chloro-6-(morpholino) | 7.72-7.76(m, 1H), 7.68(d, 1H), 7.47-7.58(m, 2H), | | |
| | pyridin-4-ylcarbonyl]-4- | 7.16-7.24(m, 2H), 7.07(s, 1H), | | |
| | methylbenzene-1,3- | 3.61-3.69(m, 4H), 3.44-3.51(m, 4H), | | |
| | diamine | 2.13(s, 3H), 1.68(s, 6H) | | |
| 15 | N¹-[3-(1-Cyano-1- | 10.29(s, 1H), 10.12(s, 1H), 8.10(s, 1H), | 598 | Method |
| | methylethyl)benzoyl]-N³- | 7.99(s, 1H), 7.89(d, 1H), 7.68(d, | | 26 |
| | [2-(4-t-butoxycarbonyl | 1H), 7.47-7.60(m, 2H), 7.21(d, 1H), | | |
| | piperazin-1-yl)-4-methyl | 7.12(s, 1H), 3.72-3.87(m, 4H), | | |
| | pyrimidin-6-ylcarbonyl]-4- | 3.31-3.40(m, 4H), 2.36(s, 3H), 2.21(s, 3H), | | |
| | methylbenzene-1,3- | 1.69(s, 6H), 1.37(s, 9H) | | |
| | diamine | | | |
| 16 | N¹-[3-(1-Cyano-1- | 10.30(s, 1H), 10.10(s, 1H), | 513 | Method |
| | methylethyl)benzoyl]-N³- | 8.15-8.20(m, 1H), 7.99(s, 1H), 7.89(d, 1H), | | 27 |
| | [2-(4-hydroxypiperidin-1- | 7.68(dd, 1H), 7.54(s, 2H), 7.20(d, | | |
| | yl)-4-methylpyrimidin-6- | 1H), 7.05(s, 1H), 4.24-4.40(m, 2H), | | |
| | ylcarbonyl]-4-methyl | 3.62-3.77(m, 1H), 3.23-3.38(m, 2H), | | |
| | benzene-1,3-diamine | 2.34(s, 3H), 2.21(s, 3H), | | |
| | | 1.70-1.80(m, 2H), 1.69(s, 6H), | | |
| | | 1.16-1.40(m, 2H) | | |
| 17 | N¹-[3-(1-Cyano-1- | 10.29(s, 1H), 10.08(s, 1H), 9.04(d, 1H), | 496 | |
| | methylethyl)benzoyl]-N³- | 8.27(dd, 1H), 7.95-8.02(m, 1H), | | |
| | {2-[2-(pyrrolidin-1- | 7.88(d, 1H), 7.80(d, 1H), 7.69(d, 1H), | | |
| | yl)ethyl]pyridin-5- | 7.43-7.59(m, 3H), 7.21(d, 1H), | | |
| | ylcarbonyl}-4-methyl | 3.43-3.64(m, 4H), 3.20(t, 2H), | | |
| | benzene-1,3-diamine | 2.95-3.13(m, 2H), 2.15(s, 3H), | | |
| | | 1.89-2.04(m, 2H), 1.75-1.89(m, 2H), 1.69(s, 6H) | | |
| 18 | N-(5-{[3-(1-Cyano-1- | 10.35(s, 1H), 10.19(s, 1H), 9.18(d, 1H), | 399 | |
| | methylethyl)benzoyl] | 8.82(dd, 1H), 8.41(dt, 1H), | | |
| | amino}-2-methylphenyl) | 8.05(t, 1H), 7.95(d, 1H), 7.86(d, 1H), | | |
| | nicotinamide | 7.71-7.80(m, 1H), 7.66(dd, 1H), | | |
| | | 7.60(t, 2H), 7.28(d, 1H), | | |
| | | 2.12-2.36(m, 3H), 1.76(s, 6H) | | |
| 19 | N-(5-{[3-(1-Cyano-1- | 10.37(s, 1H), 10.29(s, 1H), 9.32(d, 1H), | 400 | |
| | methylethyl)benzoyl] | 8.97(d, 1H), 8.84(dd, 1H), | | |
| | amino}-2-methylphenyl) | 8.14(d, 1H), 8.06(s, 1H), 7.91-8.00(m, 1H), | | |
| | pyrazine-2-carboxamide | 7.72-7.80(m, 1H), 7.60(t, 2H), | | |
| | | 7.28(d, 1H), 5.78(m, 1H), 2.28(s, 3H), | | |
| | | 1.76(s, 6H) | | |
| 20 | N-(5-{[3-(1-Cyano-1- | 10.28(s, 1H), 10.12(s, 1H), 8.95(d, | 413 | |
| | methylethyl)benzoyl] | J=1.70Hz, 1H), 8.64(d, J=1.51Hz, 1H), | | |
| | amino}-2-methylphenyl)- | 8.25(s, 1H), 7.98(t, J=1.79Hz, 1H), | | |
| | 5-methylnicotinamide | 7.88(d, J=7.72Hz, 1H), 7.79(d, | | |
| | | J=2.07Hz, 1H), 7.63-7.72(m, 1H), | | |
| | | 7.45-7.58(m, 2H), 7.21(d, J=8.48Hz, | | |
| | | 1H), 2.33-2.40(m, 3H), 2.16(s, 3H), | | |
| | | 1.68(s, 6H) | | |
| 21 | N-(5-{[3-(1-Cyano-1- | 10.33(s, 1H), 9.92(s, 1H), 8.59(d, 1H), | 443 | |
| | methylethyl)benzoyl] | 8.24(d, 1H), 8.04(s, 1H), 7.94(d, | | |
| | amino}-2-methylphenyl) | 1H), 7.83(d, 1H), 7.76(d, 1H), | | |
| | pyrazine-2,3- | 7.51-7.68(m, 2H), 7.26(d, 1H), 6.88(d, 1H), | | |
| | dicarboxamide | 2.10-2.28(m, 3H), 1.75(s, 6H) | | |
| 22 | 6-Cyano-N-(5-{[3-(1- | 10.36(s, 2H), 9.26(s, 1H), 8.55(dd, 1H), | 424 | |
| | cyano-1-methylethyl) | 8.26(d, 1H), 8.05(t, 1H), 7.95(d, | | |
| | benzoyl]amino}-2- | 1H), 7.88(d, 1H), 7.71-7.81(m, 1H), | | |
| | methylphenyl) | 7.54-7.66(m, 2H), 7.29(d, 1H), | | |
| | nicotinamide | 2.24(s, 3H), 1.75(s, 6H) | | |
| 23 | N-(5-{[3-(1-Cyano-1- | 10.24(s, 1H), 9.68(s, 1H), 8.69(d, 1H), | 484 | |
| | methylethyl)benzoyl] | 8.07(dd, 1H), 7.98(t, 1H), 7.87(d, | | |
| | amino}-2-methylphenyl)- | 1H), 7.74(d, 1H), 7.63-7.72(m, 1H), | | |
| | 6-morpholin-4- | 7.44-7.58(m, 2H), 7.18(d, 1H), | | |
| | ylnicotinamide | 6.91(d, 1H), 3.59-3.78(m, 4H), | | |
| | | 3.43-3.58(m, 4H), 2.14(s, 3H), 1.68(s, 6H) | | |
| 24 | 6-Chloro-N-(5-{[3-(1- | 10.35(s, 1H), 10.21(s, 1H), 8.98(d, 1H), | 433 | |
| | cyano-1-methylethyl) | 8.38(dd, 1H), 8.05(t, 1H), 7.94(d, | | |
| | benzoyl]amino}-2- | 1H), 7.85(d, 1H), 7.70-7.79(m, 2H), | | |
| | methylphenyl) | 7.55-7.65(m, 2H), 7.28(d, 1H), | | |
| | nicotinamide | 2.23(s, 3H), 1.77(s, 6H) | | |
| 25 | 3-Amino-N-(5-{[3-(1- | 10.34(s, 1H), 10.16(s, 1H), 8.31(d, 1H), | 415 | |
| | cyano-1-methylethyl) | 8.20(d, 1H), 8.05(s, 1H), 7.97(s, | | |
| | benzoyl]amino}-2- | 1H), 7.94(d, 2H), 7.70-7.83(m, 1H), | | |
| | methylphenyl)pyrazine-2- | 7.46-7.67(m, 3H), 7.26(d, 1H), | | |
| | carboxamide | 2.18-2.32(m, 3H), 1.76(s, 6H) | | |
| 26 | N-(5-{[3-(1-Cyano-1- | 10.37(s, 1H), 10.18(s, 1H), | 527 | Method |
| | methylethyl)benzoyl] | 8.11-8.27(m, 1H), 8.02-8.09(m, 1H), | | 42 |
| | amino}-2-methylphenyl)- | 7.96(d, 1H), 7.71-7.81(m, 1H), | | |
| | 2-[3-(hydroxymethyl) | 7.55-7.64(m, 2H), 7.28(d, 1H), 7.19(s, 1H), | | |
| | piperidin-1-yl]-6- | 4.44-4.93(m, 3H), 3.86-4.07(m, | | |
| | methylpyrimidine-4- | 2H), 3.32-3.76(m, 4H), | | |
| | carboxamide | 2.90-3.17(m, 1H), 2.64-2.89(m, 1H), 2.44(s, 3H), | | |
| | | 2.28(s, 3H), 1.76(s, 6H) | | |
| 27 | N-(5-{[3-(1-Cyano-1- | 10.31(s, 1H), 10.07(s, 1H), | 529 | Method |
| | methylethyl)benzoyl] | 8.12-8.34(m, 1H), 7.95-8.03(m, 1H), | | 43 |
| | amino}-2-methylphenyl)- | 7.89(d, 1H), 7.68(d, 1H), 7.44-7.60(m, 2H), | | |
| | 2-[2-(hydroxymethyl) | 7.21(d, 1H), 7.04(s, 1H), | | |
| | morpholin-4-yl]-6- | 4.18-4.81(m, 5H), 3.12-3.41(m, 2H), | | |
| | methylpyrimidine-4- | 2.59-3.02(m, 2H), 2.34(s, 3H), 2.24(s, 3H), | | |
| | carboxamide | 1.69(s, 6H) | | |
| 28 | N-(5-{[3-(1-Cyano-1- | 10.31(s, 1H), 10.15(s, 1H), 8.38(s, 1H), | 499 | Method |
| | methylethyl)benzoyl] | 7.99(t, 1H), 7.89(d, 1H), | | 44 |
| | amino}-2-methylphenyl)- | 7.64-7.73(m, 1H), 7.43-7.63(m, 2H), | | |
| | 2-(3-hydroxypyrrolidin-1- | 7.21(d, 1H), 7.07(s, 1H), 4.23-4.47(m, 1H), | | |
| | yl)-6-methylpyrimidine-4- | 3.49-3.60(m, 4H), 2.36(s, 3H), | | |
| | carboxamide | 2.27(s, 3H), 1.81-2.07(m, 2H), | | |
| | | 1.69(s, 6H) | | |
| 29 | N-(5-{[3-(1-Cyano-1- | 10.37(s, 1H), 10.20(s, 1H), 8.25(d, 1H), | 495 | Method |
| | methylethyl)benzoyl] | 7.99-8.10(m, 1H), 7.95(d, 1H), | | 45 |
| | amino}-2-methylphenyl)- | 7.71-7.82(m, 1H), 7.55-7.68(m, 2H), | | |
| | 2-(3,6-dihydropyridin- | 7.28(d, 1H), 7.16(s, 1H), | | |
| | 1(2H)-yl)-6-methyl | 5.60-6.08(m, 2H), 4.19-4.50(m, 2H), | | |
| | pyrimidine-4-carboxamide | 3.82-4.12(m, 2H), 2 2.47(s, 3H), 2.30(s, | | |
| | | 3H), 2.19-2.29(m, 2H), 1.76(s, 6H) | | |
| 30 | N-(5-{[3-(1-Cyano-1- | 10.35(s, 1H), 10.12(s, 1H), 8.34(s, 1H), | 540 | Method |
| | methylethyl)benzoyl] | 8.06(s, 1H), 7.96(d, 1H), 7.75(d, | | 46 |
| | amino}-2-methylphenyl)- | 1H), 7.59(t, 2H), 7.26(d, 1H), | | |
| | 6-(cyclopropylamino)-2- | 3.55-4.00(m, 8H), 2.67-2.96(m, 1H), | | |
| | morpholin-4-ylpyrimidine- | 2.26(s, 3H), 1.76(s, 6H), 0.66-0.86(m, 2H), | | |
| | 4-carboxamide | 0.36-0.57(m, 2H) | | |
| 31 | N-(5-{[3-(1-Cyano-1- | 10.36(s, 1H), 10.15(s, 1H), 8.28(d, 1H), | 570 | Method |
| | methylethyl)benzoyl] | 8.02-8.10(m, 1H), 7.95(d, 1H), | | 47 |
| | amino}-2-methylphenyl)- | 7.72-7.80(m, 1H), 7.60(t, 2H), | | |
| | 2,6-dimorpholin-4- | 7.26(d, 1H), 6.77(s, 1H), 3.34-3.92(m, | | |
| | ylpyrimidine-4- | 16H), 2.28(s, 3H), 1.76(s, 6H) | | |
| | carboxamide | | | |
| 32 | N-(5-{[3-(1-Cyano-1- | 10.29(s, 1H), 10.07(s, 1H), 8.23(d, 1H), | 568 | Method |
| | methylethyl)benzoyl] | 7.98(s, 1H), 7.88(d, 1H), 7.68(d, | | 48 |
| | amino}-2-methylphenyl)- | 1H), 7.52(t, 2H), 7.19(d, 1H), 6.69(s, | | |
| | 2-morpholin-4-yl-6- | 1H), 3.10-3.82(m, 12H), 2.21(s, 3H), | | |
| | piperidin-1-ylpyrimidine- | 1.69(s, 6H), 1.38-1.62(m, 6H) | | |
| | 4-carboxamide | | | |
| 33 | N-(5-{[3-(1-Cyano-1- | 10.41(s, 1H), 10.17(s, 1H), 9.35(s, 1H), | 567 | Method |
| | methylethyl)benzoyl] | 8.25(s, 1H), 8.07(s, 1H), 7.96(d, | | 40 |
| | amino}-2-methylphenyl)- | 1H), 7.74(d, 1H), 7.60(t, 2H), | | |
| | 2-morpholin-4-yl-6- | 7.26(d, 1H), 6.85(s, 1H), 3.01-4.23(m, | | |
| | piperazin-1-ylpyrimidine- | 16H), 2.26(s, 3H), 1.76(s, 6H) | | |
| | 4-carboxamide | | | |
| 34 | N-(5-{[3-(1-Cyano-1- | 10.37(s, 1H), 10.17(s, 1H), 8.25(d, 1H), | 527 | Method |
| | methylethyl)benzoyl] | 8.06(s, 1H), 7.95(d, 1H), 7.74(d, | | 37 |
| | amino}-2-methylphenyl)- | 1H), 7.58-7.69(m, 2H), 7.28(d, 1H), | | |
| | 2-[4-(hydroxymethyl) | 7.11(s, 1H), 4.73-5.03(m, 2H), | | |
| | piperidin-1-yl]-6-methyl | 2.85-3.32(m, 4H), 2.39-2.45(m, 3H), | | |
| | pyrimidine-4-carboxamide | 2.28(s, 3H), 1.69-1.88(m, 7H), | | |
| | | 1.08-1.35(m, 4H) | | |
| 35 | N-(5-{[3-(1-Cyano-1- | 10.37(s, 1H), 10.17(s, 1H), 8.31(s, 1H), | 558 | Method |
| | methylethyl)benzoyl] | 8.06(s, 1H), 7.96(d, 1H), 7.75(d, | | 39 |
| | amino}-2-methylphenyl)- | 1H), 7.59(t, 2H), 7.26(d, 1H), 6.72(s, | | |
| | 6-[(2-hydroxyethyl) | 1H), 4.51-4.90(m, 9H), | | |
| | (methyl)amino]-2- | 3.45-4.04(m, 3H), 3.13(s, 3H), 2.28(s, 3H), | | |
| | morpholin-4-ylpyrimidine- | 1.76(s, 6H) | | |
| | 4-carboxamide | | | |
| 36 | N-(5-{[3-(1-Cyano-1- | 10.35(s, 1H), 10.12(s, 1H), 8.35(s, 1H), | 514 | Method |
| | methylethyl)benzoyl] | 8.06(t, 1H), 7.96(d, 1H), 7.75(d, | | 38 |
| | amino}-2-methylphenyl)- | 1H), 7.59(t, 2H), 7.25(d, 1H), 6.55(s, | | |
| | 6-(methylamino)-2- | 1H), 3.49-3.92(m, 8H), 2.84(s, 3H), | | |
| | morpholin-4-ylpyrimidine- | 2.28(s, 3H), 1.73(s, 6H) | | |
| | 4-carboxamide | | | |
| 37 | N-(5-{[3-(1-Cyano-1- | 10.36(s, 1H), 10.09(s, 1H), 8.64(s, 1H), | 583 | Method |
| | methylethyl)benzoyl] | 8.37(s, 1H), 8.06(t, 1H), 7.94(d, 1H), | | 41 |
| | amino}-2-methylphenyl)- | 7.67-7.82(m, 2H), 7.51-7.64(m, | | |
| | 2-morpholin-4-yl-6- | 2H), 7.26(d, 1H), 6.59(s, 1H), | | |
| | (piperidin-4-ylamino) | 3.97-4.28(m, 1H), 3.63-3.95(m, 8H), | | |
| | pyrimidine-4-carboxamide | 2.96-3.36(m, 4H), 2.28(s, 3H), | | |
| | | 1.97-2.15(m, 2H), 1.76(s, 6H), | | |
| | | 1.54-1.69(m, 1H), 1.19-1.45(m, 1H) | | |
| 38 | N-(5-{[3-(1-Cyano-1- | 10.38(s, 1H), 10.11(s, 1H), 8.33(s, 1H), | 571 | Method |
| | methylethyl)benzoyl] | 8.06(t, 1H), 7.96(d, 1H), 7.75(d, | | 36 |
| | amino}-2-methylphenyl)- | 1H), 7.49-7.67(m, 2H), 7.26(d, 1H), | | |
| | 6-{[2-(dimethylamino) | 6.60(d, 1H), 3.56-3.89(m, 8H), | | |
| | ethyl]amino}-2-morpholin- | 3.07-3.25(m, 4H), 2.81(d, 3H), 2.27(s, 3H), | | |
| | 4-ylpyrimidine-4- | 1.77(s, 6H) | | |
| | carboxamide | | | |
| 39 | N-(5-{[3-(1-Cyano-1- | 10.35(s, 1H), 10.11(s, 1H), 8.33(s, 1H), | 570 | Method |
| | methylethyl)benzoyl]amino}- | 8.06(s, 1H), 7.94(d, 1H), 7.76(d, | | 35 |
| | 2-methylphenyl)-6- | 1H), 7.61(t, 2H), 7.27(d, 1H), 6.54(s, | | |
| | {[(1RS,2SR)-2- | 1H), 3.24-4.02(m, 11H), 2.28(s, 3H), | | |
| | (hydroxymethyl) | 1.76(s, 6H), 1.09-1.30(m, 2H), | | |
| | cyclopropyl]amino}-2- | 0.89-1.00(m, 1H) | | |
| | morpholin-4-ylpyrimidine- | | | |
| | 4-carboxamide | | | |
| 40 | N-(5-{[3-(1-Cyano-1- | 10.36(s, 1H), 9.98(s, 1H), 8.87(s, 1H), | 553 | Method |
| | methylethyl)benzoyl] | 8.05(s, 1H), 7.87-8.00(m, 2H), | | 34 |
| | amino}-2-methylphenyl)- | 7.75(d, 1H), 7.60(t, 2H), 7.25(d, 1H), | | |
| | 2-morpholin-4-yl-4- | 3.57-3.93(m, 8H), 2.24(s, 3H), | | |
| | (trifluoromethyl) | 1.76(s, 6H) | | |
| | pyrimidine-5-carboxamide | | | |
| 41 | N-(5-{[3-(1-Cyano-1- | 10.35(s, 1H), 9.99(s, 1H), 8.88(s, 1H), | 499 | Method |
| | methylethyl)benzoyl] | 8.01(s, 1H), 7.82-7.94(m, 2H), | | 33 |
| | amino}-2-methylphenyl)- | 7.78(d, 1H), 7.66(t, 2H), 7.23(d, 1H), | | |
| | 4-methyl-2-morpholin-4- | 3.57-3.93(m, 8H), 2.27(s, 3H), | | |
| | ylpyrimidine-5- | 2.23(s, 3H), 1.77(s, 6H) | | |
| | carboxamide | | | |
| 42 | N-(5-{[3-(1-Cyano-1- | 10.29(s, 1H), 9.70(s, 1H), 8.74(s, 1H), | 540 | Method |
| | methylethyl)benzoyl] | 8.04(s, 1H), 7.92(d, 1H), | | 49 |
| | amino}-2-methylphenyl)- | 7.71-7.83(m, 2H), 7.48-7.65(m, 2H), | | |
| | 4-(cyclopropylamino)-2- | 7.25(d, 1H), 3.65-3.92(m, 8H), | | |
| | morpholin-4-ylpyrimidine- | 2.84-2.99(m, 1H), 2.18(s, 3H), 1.75(s, 6H), | | |
| | 5-carboxamide | 0.65-0.84(m, 2H), 0.30-0.61(m, | | |
| | | 2H) | | |
| 43 | N-(5-{[3-(1-Cyano-1- | 10.68(s, 1H), 10.39(s, 1H), 9.85(d, 1H), | 491 | Method |
| | methylethyl)benzoyl] | 9.04(dt, 1H), 8.81(dd, 1H), | | 70 |
| | amino}-2-methylphenyl)- | 8.11(d, 1H), 8.06(s, 1H), 8.03(s, 1H), | | |
| | 6-methyl-2-pyridin-3- | 7.96(d, 1H), 7.76(d, 1H), | | |
| | ylpyrimidine-4- | 7.55-7.72(m, 3H), 7.33(d, 1H), 2.69-2.81(m, 3H), | | |
| | carboxamide | 2.32(s, 3H), 1.78(s, 6H) | | |
| 44 | N-(5-{[3-(1-Cyano-1- | 10.69(s, 1H), 10.40(s, 1H), 8.89(d, 2H), | 491 | Method |
| | methylethyl)benzoyl] | 8.68(d, 1H), 8.11(s, 1H), 8.06(s, | | 69 |
| | amino}-2-methylphenyl)- | 1H), 7.94-7.99(m, 1H), 7.76(d, 1H), | | |
| | 6-methyl-2-pyridin-4- | 7.56-7.69(m, 3H), 7.33(d, 1H), | | |
| | ylpyrimidine-4- | 2.74(s, 3H), 2.30(s, 3H), 1.76(s, 6H) | | |
| | carboxamide | | | |
| 45 | N⁵-(5-{[3-(1-Cyano-1- | 10.34(s, 1H), 9.37(s, 1H), | 456 | Method |
| | methylethyl)benzoyl] | 7.92-8.02(m, 2H), 7.82-7.89(m, 1H), | | 85 |
| | amino}-2-methylphenyl)- | 7.62-7.75(m, 2H), 7.43-7.60(m, 3H), | | |
| | N²-methylpyridine-2,5- | 7.18-7.36(m, 2H), 2.79(s, 3H), 2.19(s, 3H), | | |
| | dicarboxamide | 1.69(s, 6H) | | |
| 46 | N-(5-{[3-(1-Cyano-1- | 10.33(s, 1H), 8.41(s, 1H), 8.03(s, 1H), | 539 | Method |
| | methylethyl)benzoyl] | 7.93(d, 1H), 7.81(s, 1H), | | 84 |
| | amino}-2-methylphenyl)- | 7.71-7.79(m, 1H), 7.45-7.67(m, 2H), | | |
| | 4-(cyclopropylamino)-6- | 7.26(d, 1H), 6.29(s, 1H), 3.60-3.96(m, 8H), | | |
| | morpholin-4-ylpyridine-2- | 2.57-2.67(m, 1H), 2.19(s, 3H), | | |
| | carboxamide | 1.75(s, 6H), 0.77-1.07(m, 2H), | | |
| | | 0.43-0.70(m, 2H) | | |
| 47 | N-(5-{[3-(1-Cyano-1- | 10.33(s, 1H), 10.12(s, 1H), 8.43(d, 1H), | 669 | Method |
| | methylethyl)-5-(2- | 8.32(s, 1H), 7.72(s, 1H), | | 46 |
| | morpholin-4-ylethoxy) | 7.49-7.61(m, 2H), 7.19-7.38(m, 2H), | | |
| | benzoyl]amino}-2- | 2.95-3.90(m, 21H), 2.28(s, 3H), 1.76(s, 6H), | | |
| | methylphenyl)-6- | 1.02-1.42(m, 2H), 0.35-0.91(m, | | |
| | (cyclopropylamino)-2- | 2H) | | |
| | morpholin-4-ylpyrimidine- | | | |
| | 4-carboxamide | | | |

### Examples 48-58

The following compounds were prepared by the procedure of Example 1, using the appropriate carboxylic acid (commercially available unless otherwise indicated) and N-(3-amino-4-methylphenyl)-3-(trifluoromethyl)benzamide (Method 66) as a starting materials.

| Ex | Compound | NMR | m/z | SM |
|---|---|---|---|---|
| 48 | N¹-(3-Trifluoromethyl | 10.46(s, 1H), 10.13(s, 1H), 8.17-8.28(m, | 500 | Method |
| | benzoyl)-N³-[2- | 2H), 8.12(d, 1H), 7.91(d, 1H), 7.72(t, 1H), | | 22 |
| | (morpholino)-4- | 7.57(dd, 1H), 7.21(d, 1H), 7.12(s, 1H), | | |
| | methylpyrimidin-6- | 3.71-3.84(m, 4H), 3.58-3.68(m, 4H), | | |
| | ylcarbonyl]-4-methyl | 2.36(s, 3H), 2.20(s, 3H) | | |
| | benzene-1,3-diamine | | | |
| 49 | N¹-(3-Trifluoromethyl | 10.47(s, 1H), 10.21(s, 1H), 8.16-8.28(m, | 513 | Method |
| | benzoyl)-N³-[2-(4- | 2H), 8.04(d, 1H), 7.91(d, 1H), 7.73(t, 1H), | | 21 |
| | methylpiperazin-1-yl)- | 7.56(dd, 1H), 7.19-7.29(m, 2H), | | |
| | 4-methylpyrimidin-6- | 4.81-4.98(m, 2H), 3.40-3.56(m, 2H), | | |
| | ylcarbonyl]-4-methyl | 3.12-3.32(m, 2H), 2.93-3.12(m, 2H), 2.79(s, | | |
| | benzene-1,3-diamine | 3H), 2.40(s, 3H), 2.19(s, 3H) | | |
| 50 | N¹-(3-Trifluoromethyl | 10.46(s, 1H), 10.23(s, 1H), 9.25(d, 1H), | 401 | |
| | benzoyl)-N³-[pyrazin- | 8.90(d, 1H), 8.72-8.80(m, 1H), | | |
| | 2-ylcarbonyl]-4- | 8.16-8.29(m, 2H), 8.10(d, 1H), 7.90(d, 1H), | | |
| | methylbenzene-1,3- | 7.72(t, 1H), 7.56(dd, 1H), 7.22(d, 1H), | | |
| | diamine | 2.21(s, 3H) | | |
| 51 | N¹-(3-Trifluoromethyl | 10.45(s, 1H), 10.23(s, 1H), 9.32(s, 1H), | 401 | |
| | benzoyl)-N³- | 9.23(s, 2H), 8.16-8.26(m, 2H), 7.92(d, 1H), | | |
| | [pyrimidin-5- | 7.85(d, 1H), 7.72(t, 1H), 7.55(dd, 1H), | | |
| | ylcarbonyl]-4-methyl | 7.23(d, 1H), 2.18(s, 3H) | | |
| | benzene-1,3-diamine | | | |
| 52 | N¹-(3-Trifluoromethyl | 10.46(s, 1H), 10.22(s, 1H), 8.64-8.73(m, | 400 | |
| | benzoyl)-N³-[pyridin- | 1H), 8.17-8.31(m, 3H), 8.08-8.17(m, 1H), | | |
| | 2-ylcarbonyl]-4-methyl | 7.97-8.08(m, 1H), 7.90(d, 1H), | | |
| | benzene-1,3-diamine | 7.72(t, 1H), 7.59-7.68(m, 1H), 7.55(dd, 1H), | | |
| | | 7.21(d, 1H), 2.24(s, 3H) | | |
| 53 | N¹-(3-Trifluoromethyl | 10.45(s, 1H), 10.26(s, 1H), 8.81(d, 2H), | 400 | |
| | benzoyl)-N³-[pyridin- | 8.16-8.27(m, 2H), 7.87-7.98(m, 3H), | | |
| | 4-ylcarbonyl]-4-methyl | 7.82(d, 1H), 7.72(t, 1H), 7.56(dd, 1H), | | |
| | benzene-1,3-diamine | 7.23(d, 1H), 2.16(s, 3H) | | |
| 54 | N¹-(3-Trifluoromethyl | 10.44(s, 1H), 10.13(s, 1H), 9.12(d, 1H), | 400 | |
| | benzoyl)-N³-[pyridin- | 8.75(dd, 1H), 8.35(d, 1H), 8.14-8.29(m, | | |
| | 3-ylcarbonyl]-4-methyl | 2H), 7.90(d, 1H), 7.82(d, 1H), 7.72(t, 1H), | | |
| | benzene-1,3-diamine | 7.46-7.65(m, 2H), 7.22(d, 1H), | | |
| | | 2.17(s, 3H) | | |
| 55 | N¹-(3-Trifluoromethyl | 10.46(s, 1H), 10.23(s, 1H), 8.36(d, 1H), | 414 | |
| | benzoyl)-N³-[6-methyl | 8.17-8.28(m, 2H), 7.84-7.94(m, 3H), | | |
| | pyridin-2-ylcarbonyl]- | 7.72(t, 1H), 7.45-7.58(m, 2H), 7.21(d, 1H), | | |
| | 4-methyl benzene-1,3- | 2.56(s, 3H), 2.26(s, 3H) | | |
| | diamine | | | |
| 56 | N¹-(3-Trifluoromethyl | 10.49(s, 1H), 10.17(s, 1H), 9.38(s, 1H), | 503 | |
| | benzoyl)-N³-(4- | 8.18-8.26(m, 2H), 7.97(d, 1H), 7.91(d, 1H), | | |
| | trifluoromethyl-5- | 7.72(t, 1H), 7.59(dd, 1H), 7.22(d, 1H), | | |
| | chloropyrimidin-2- | 2.19(s, 3H) | | |
| | ylcarbonyl)-4-methyl | | | |
| | benzene-1,3-diamine | | | |
| 57 | N¹-(3-Trifluoromethyl | 10.50(s, 1H), 10.11(s, 1H), 8.95(s, 1H), | 414 | |
| | benzoyl)-N³-(3-methyl | 8.63(s, 1H), 8.31(s, 1H), 8.28(d, J=7.9Hz, | | |
| | pyridin-5-ylcarbonyl)- | 1H), 8.15(s, 1H), 7.98(d, J=7.7Hz, 1H), | | |
| | 4-methyl benzene-1,3- | 7.86(s, 1H), 7.79(t, J=7.8Hz, 1H), | | |
| | diamine | 7.60-7.64(m, 1H), 7.28(d, J=8.4Hz, 1H), | | |
| | | 2.41(s, 3H), 2.23(s, 3H) | | |
| 58 | 6-(Cyclopropylamino)- | 10.51(s, 1H), 10.12(s, 1H), 8.22-8.40(m, | 541 | Method |
| | N-(2-methyl-5-{[3- | 3H), 7.91-8.02(m, 1H), 7.49-7.84(m, 3H), | | 46 |
| | (trifluoromethyl)benzoyl] | 7.17-7.34(m, 1H), 3.59-3.88(m, 8H), | | |
| | amino}phenyl)-2- | 2.29(s, 3H), 1.08-1.23(m, 1H), | | |
| | morpholin-4- | 0.67-0.80(m, 2H), 0.35-0.58(m, 2H) | | |
| | ylpyrimidine-4- | | | |
| | carboxamide | | | |

### Example 59

### N¹-[3-(1-Cyano-1-methylethyl)benzoyl]-N³-[2-(morpholino)pyrimidin-6-ylcarbonyl]-4-methylbenzene-1,3-diamine

To a stirring solution of N′-[3-(1-cyano-1-methylethyl)benzoyl]-N³-[2-(morpholino)-5-chloropyrimidin-6-ylcarbonyl]-4-methylbenzene-1,3-diamine (Example 3; 0.050 g, 0.096 mmol) in 20 ml anhydrous EtOAc and 0.08 ml triethylamine was added palladium, 10 wt. % on activated carbon (0.005 g, 0.005 mmol) and the reaction mixture was allowed to stir at 25° C. for 6 hours. The reaction mixture was filtered over diatomaceous earth and the filtrate was concentrated under reduced pressure. The resulting residue was purified by reverse phase semi-preparative chromatography. NMR (400 MHz): 10.29 (s, 1H), 10.15 (s, 1H), 8.61 (d, 1H), 8.06-8.11 (m, 1H), 7.96-8.01 (m, 1H), 7.85-7.91 (m, 1H), 7.65-7.72 (m, 1H), 7.48-7.60 (m, 2H), 7.16-7.25 (m, 2H), 3.74-3.84 (m, 4H), 3.58-3.69 (m, 4H), 2.19 (s, 3H), 1.69 (s, 6H); m/z 485.

### Example 60

### N-(5-{[3-(1-Cyano-1-methylethyl)benzoyl]amino}-2-methylphenyl)-6-morpholin-4-ylpyrimidine-4-carboxamide

The title compound was prepared by the procedure of Example 59, using N′-[3-(1-cyano-1-methylethyl)benzoyl]-N³-[2-chloro-4-(morpholino) pyrimidin-6-ylcarbonyl]-4-methylbenzene-1,3-diamine (Example 10) as a starting material. NMR (400 MHz): 10.31 (s, 1H), 10.22 (s, 1H), 8.64 (s, 1H), 8.17 (d, 1H), 7.98 (s, 1H), 7.88 (d, 1H), 7.68 (d, 1H), 7.52 (s, 2H), 7.41 (s, 1H), 7.20 (d, 1H), 3.60-3.73 (m, 8H), 2.20 (s, 3H), 1.69 (s, 6H); m/z 485.

### Example 61

### N-(5-{[3-(1-Cyano-1-methylethyl)benzoyl]amino}-2-methylphenyl)-6-methyl-2-piperazin-1-ylpyrimidine-4-carboxamide

N¹-[3-(1-Cyano-1-methylethyl)benzoyl]-N³-[2-(4-t-butoxycarbonyl piperizin-1-yl)-4-methylpyrimidin-6-ylcarbonyl]-4-methylbenzene-1,3-diamine (Example 15; 0.150 g, 0.25 mmol) in 10 ml 4 N HCl in dioxane was allowed to stir at 25° C. for 3 hours. The reaction mixture was concentrated under reduced pressure to give the title compound. NMR (400 MHz): 10.39 (s, 1H), 10.24 (s, 1H), 8.13 (d, 1H), 8.03-8.08 (m, 1H), 7.96 (d, 1H), 7.76 (d, 1H), 7.54-7.66 (m, 2H), 7.21-7.35 (m, 2H), 3.98-4.19 (m, 4H), 3.13-3.29 (m, 4H), 2.46 (s, 3H), 2.26 (s, 3H), 1.76 (s, 6H); m/z 498.

### Example 62

### N-(5-{[3-(1-Cyano-1-methylethyl)benzoyl]amino}-2-methylphenyl)-6-{[2-(dimethylamino)ethyl]amino}nicotinamide

To a stirring solution of 6-chloro-N-(5-{[3-(1-cyano-1-methylethyl)benzoyl]amino}-2-methylphenyl)nicotinamide (Example 24; 50 mg, 0.115 mmol) in 2 ml of anhydrous EtOH were added sequentially Et₃N (0.1 ml) and N,N-dimethylethylenediamine (0.1 ml, 0.911 mmol). The resulting solution was heated to 70° C. for 16 hours. Evaporation of the volatiles and purification by reverse phase semi-preparative chromatography (5-95% acetonitrile/H₂O, 15 min) afforded the title compound (20 mg). NMR (300 MHz): 10.37 (s, 1H), 9.95 (s, 1H), 8.71 (d, 1H), 8.19 (d, 1H), 8.06 (t, 1H), 7.95 (d, 1H), 7.82 (d, 1H), 7.70-7.79 (m, 1H), 7.54-7.65 (m, 2H), 7.25 (d, 1H), 6.86 (d, 1H), 3.01-3.19 (m, 4H), 2.80 (s, 6H), 2.20 (s, 3H), 1.74 (s, 6H); m/z 485.

### Examples 63-66

The following compounds were prepared by the procedure of Example 62, using the appropriate amine (commercially available) and 6-chloro-N-(5-{[3-(1-cyano-1-methylethyl)benzoyl]amino}-2-methylphenyl)nicotinamide (Example 24) as starting materials.

| Ex | Compound | NMR | m/z | SM |
|---|---|---|---|---|
| 63 | N-(5-{[3-(1-Cyano-1- | 10.33(s, 1H), 9.90(s, 1H), 8.56(d, 1H), | 458 | 2-hydroxy |
| | methylethyl)benzoyl] | 8.16(d, 1H), 8.04(d, 1H), | | ethylamine |
| | amino}-2-methyl | 7.94(d, 1H), 7.84(d, 1H), 7.75(dd, 1H), | | |
| | phenyl)-6-[(2- | 7.49-7.67(m, 2H), 7.26(d, 1H), | | |
| | hydroxyethyl)amino] | 6.95(d, 1H), 3.54-3.80(m, 2H), | | |
| | nicotinamide | 3.36-3.46(m, 2H), 2.20(s, 3H), | | |
| | | 1.75(s, 6H) | | |
| 64 | N-(5-{[3-(1-Cyano-1- | 10.31(s, 1H), 9.74(s, 1H), 8.77(d, 1H), | 514 | 2- |
| | methylethyl)benzoyl] | 8.13(dd, 1H), 8.04(d, 1H), | | hydroxymethyl |
| | amino}-2-methyl | 7.94(d, 1H), 7.81(d, 1H), 7.75(d, 1H), | | morpholine |
| | phenyl)-6-[2- | 7.54-7.66(m, 2H), 7.25(d, 1H), | | |
| | (hydroxymethyl) | 6.95(d, 1H), 4.37(d, 1H), 4.19(d, 2H), | | |
| | morpholin-4- | 3.40-3.62(m, 5H), 2.97(ddd, 1H), | | |
| | yl]nicotinamide | 2.65-2.80(m, 1H), 2.21(s, 3H), | | |
| | | 1.75(s, 6H) | | |
| 65 | N-(5-{[3-(1-Cyano-1- | 10.26(s, 1H), 10.21(s, 1H), 8.98(d, | 496 | 5-amino- |
| | methylethyl)benzoyl] | 1H), 8.37(dd, 1H), 7.80-7.94(m, 3H), | | 1,2-dihydro- |
| | amino}-2-methyl | 7.73(d, 1H), 7.60(dd, 1H), | | 3H-pyrazol- |
| | phenyl)-6-[(5-oxo-2,5- | 7.43-7.58(m, 2H), 7.27(d, 1H), | | 3-one |
| | dihydro-1H-pyrazol-3- | 6.91-7.00(m, 2H), 2.16(s, 3H), 1.77(s, 1H), | | |
| | yl)amino]nicotinamide | 1.49(s, 6H) | | |
| 66 | 6-[(4-Cyano-1H- | 10.24(s, 1H), 10.20(s, 1H), 8.99(d, | 505 | 4-amino-5- |
| | imidazol-5-yl)amino]- | 1H), 8.30-8.26(m, 1H), | | cyano-1H- |
| | N-(5-{[3-(1-cyano-1- | 7.80-7.94(m, 3H), 7.66-7.77(m, 2H), 7.58(dd, | | imidazole |
| | methylethyl)benzoyl] | 1H), 7.46-7.57(m, 2H), 7.26(d, 1H), | | |
| | amino}-2-methyl | 6.91-7.04(m, 2H), 2.15(s, 3H), | | |
| | phenyl)nicotinamide | 1.50(s, 6H) | | |

### Example 67

### 6-(Aminomethyl)-N-(5-{[3-(1-cyano-1-methylethyl)benzoyl]amino}-2-methylphenyl)nicotinamide

To a solution of 6-cyano-N-(5-{[3-(1-cyano-1-methylethyl)benzoyl]amino}-2-methylphenyl)nicotinamide (Example 22; 46.4 mg, 0.110 mmol) in 2 ml of anhydrous THF at 0° C. was added slowly a solution of LiAlH₄ (0.5 ml, 1M in THF). After the evolution of gas ceased the resulting mixture was warmed to 25° C. and stirred for 10 hours. The reaction was cooled to 0° C. and treated cautiously with 1M solution of tartaric acid until a precipitate was formed. The solid was filtered and the cake washed excessively with EtOAc. Evaporation of the volatiles afforded a yellow oil which was purified by reverse phase semi-preparative chromatography. NMR (300 MHz): 10.29 (s, 1H), 10.15 (s, 1H), 9.11 (d, 1H), 8.20-8.44 (m, 3H), 7.98 (t, 1H), 7.79-7.93 (m, 2H), 7.69 (d, 1H), 7.44-7.62 (m, 3H), 7.22 (d, 1H), 4.21-4.29 (m, 2H), 2.16 (s, 3H), 1.69 (s, 6H); m/z 428.

### Example 68

### N-(5-j{[3-(1-Cyano-1-methylethyl)benzoyl]amino}-2-methylphenyl)-6-(1H-pyrazol-4-yl)nicotinamide

To a solution of 6-chloro-N-(5-{[3-(1-cyano-1-methylethyl)benzoyl]amino}-2-methylphenyl)nicotinamide (Example 24; 50 mg, 0.115 mmol) in 5 ml of dioxane/H₂O (4:1 v/v), in a microwave tube, was added Cs₂CO₃ (100 mg, 0.307 mmol), Pd(PPh₃)₄ (3 mg, 0.0033 mmol) and 4-(4,4,5,5-tetramethyl-1,3,2-dioxaborolan-2-yl)-1H-pyrazole (50 mg, 0.258 mmol). The resulting solution was heated to 150° C. in a SmithSynthesizer for 20 min. The mixture was partitioned between EtOAc and H₂O. The organic layer washed with brine, H₂O and dried (MgSO₄). Evaporation of the volatiles afforded a brown oil which was purified by reverse phase semi-preparative chromatography. NMR (300 MHz): 10.27 (s, 1H), 9.99 (s, 1H), 9.02 (d, 1H), 8.19-8.29 (m, 3H), 7.96-8.01 (m, 1H), 7.88 (d, 1H), 7.75-7.84 (m, 2H), 7.63-7.71 (m, 1H), 7.44-7.61 (m, 2H), 7.21 (d, 1H), 2.18 (s, 3H), 1.69 (s, 6H); m/z 465.

### Example 69

The following compound was prepared by the procedure of Example 68, using the appropriate boronic acid or boronic ester (commercially available) and 5-bromo-N-(5-{[3-(1-cyano-1-methylethyl)benzoyl]amino}-2-methylphenyl)-2-morpholin-4-ylpyrimidine-4-carboxamide (Example 81) as starting materials.

| Ex | Compound | NMR | m/z | SM |
|---|---|---|---|---|
| 69 | N-(5-{[3-(1-Cyano-1- | 10.35(s, 1H), 10.08(d, 1H), 8.70(d, | 551 | 4-(4,4,5,5- |
| | methylethyl)benzoyl] | 1H), 8.01-8.09(m, 1H), | | tetramethyl- |
| | amino}-2-methyl | 7.91-7.99(m, 2H), 7.80-7.85(m, 1H), | | 1,3,2- |
| | phenyl)-2-morpholin-4- | 7.71-7.79(m, 1H), 7.52-7.68(m, 3H), | | dioxaborolan- |
| | yl-5-(1H-pyrazol-4- | 7.25(d, 1H), 3.64-3.92(m, 8H), | | 2-yl)-1H- |
| | yl)pyrimidine-4- | 2.13(d, 3H), 1.76(s, 6H) | | pyrazole |
| | carboxamide | | | |

### Example 70

### 5-{[(5-{[3-(1-Cyano-1-methylethyl)benzoyl]amino}-2-methylphenyl)amino]carbonyl}nicotinic acid

N-(3-Amino-4-methylphenyl)-3-(1-cyano-1-methylethyl)benzamide (Method 59; 100 mg, 0.34 mmol), 5-carboxy nicotinic acid (57 mg, 0.34 mmol), HATU (193 mg, 0.51 mmol) and DIEA (0.18 ml, 1.02 mmol) were combined in 2 ml anhydrous DMF and the reaction mixture was stirred at 25° C. for 15 hours. The reaction mixture was concentrated under reduced pressure and purified by reverse phase semi-preparative chromatography; m/z 443.

### Example 71

### N-(5-{[3-(1-Cyano-1-methylethyl)benzoyl]amino}-2-methylphenyl)-N′-methylpyridine-3,5-dicarboxamide

5-{[(5-{[3-(1-Cyano-1-methylethyl)benzoyl]amino}-2-methylphenyl)amino]carbonyl}nicotinic acid (Example 70; 50 mg, 0.11 mmol), methylamine hydrochloride (100 mg, 0.34 mmol), HATU (100 mg, 0.51 mmol) and DIEA (0.2 ml, 1.02 mmol) were combined in 1 ml anhydrous DMF and the reaction mixture was stirred at 25° C. for 15 hours. The reaction mixture was then concentrated under reduced pressure and purified by reverse phase semi-preparative chromatography. NMR (300 MHz): 10.35 (s, 1H), 10.28 (s, 1H), 9.25 (d, 1H), 9.16 (d, 1H), 8.79-8.92 (m, 1H), 8.71 (t, 1H), 8.03-8.07 (m, 1H), 7.82-7.98 (m, 2H), 7.74 (d, 1H), 7.54-7.65 (m, 1H), 7.30 (d, 1H), 6.16 (d, 1H), 2.85 (d, 3H), 2.24 (s, 3H), 1.77 (s, 6H); m/z 456.

### Example 72

The following compound was prepared by the procedure of Example 71, using the appropriate amine and 5-{[(5-{[3-(1-cyano-1-methylethyl)benzoyl]amino}-2-methylphenyl)amino]-carbonyl}nicotinic acid (Example 70) as starting materials.

| Ex | Compound | NMR | m/z | SM |
|---|---|---|---|---|
| 72 | N-(5-{[3-(1-Cyano-1- | 10.35(s, 1H), 10.27(s, 1H), 9.23(d, 1H), | 482 | cyclopropyl- |
| | methylethyl)benzoyl] | 9.13(d, 1H), 8.83(d, 1H), 8.68(t, 1H), | | amine |
| | amino}-2- | 8.00-8.08(m, 1H), 7.94(d, 1H), | | |
| | methylphenyl)-N′- | 7.83-7.87(m, 1H), 7.74(d, 1H), | | |
| | cyclopropylpyridine- | 7.55-7.65(m, 2H), 7.30(d, 1H), | | |
| | 3,5-dicarboxamide | 2.22-2.24(m, 3H), 1.76(s, 6H), 1.16-1.26(m, 3H), | | |
| | | 0.54-0.84(m, 2H) | | |

### Example 73

### N-(5-{[3-(1-Cyano-1-methylethyl)benzoyl]amino}-2-methylphenyl)-6-morpholin-4-ylpyridine-2-carboxamide

N-(3-Amino-4-methylphenyl)-3-(1-cyano-1-methylethyl)benzamide (Method 59; 100 mg, 0.34 mmol), 6-chloro picolinic acid (mg, 0.34 mmol), HATU (193 mg, 0.51 mmol) and DIEA (0.18 ml, 1.02 mmol) were combined in 5 ml anhydrous DMF and the reaction mixture was stirred at 25° C. for 15 hours. The reaction mixture was concentrated under, reduced pressure and used in the next step without further purification. This compound was dissolved in 5 ml NMP and morpholine (0.210 ml, 2.41 mmol) was added in a microwave tube. The reaction was heated in Smith™ Personal Chemistry Microwave at 160° C. for 2200 seconds. Purification by reverse phase semi-preparative chromatography (5-95% acetonitrile/H₂O, 15 min) afforded the title compound. NMR (300 MHz): 10.42 (s, 1H), 10.17 (s, 1H), 8.43 (s, 1H), 7.97-8.30 (m, 2H), 7.76-7.92 (m, 2H), 7.60-7.73 (m, 2H), 7.53 (d, 1H), 7.32 (d, 1H), 7.21 (d, 1H), 3.59-4.00 (m, 8H), 2.36 (s, 3H), 1.82 (s, 6H); m/z 484.

### Example 74

### N-(5-{[3-(1-Cyano-1-methylethyl)benzoyl]amino}-2-methylphenyl)-3-[(1H-imidazol-2-ylmethyl)amino]pyrazine-2-carboxamide

To a solution of 3-amino-N-(5-{[3-(1-cyano-1-methylethyl)benzoyl]amino}-2-methylphenyl)pyrazine-2-carboxamide (Example 25; 40 mg, 0.097 mmol) in 0.5 ml of anhydrous THF at ambient temperature were added imidazole 2-carboxaldehyde (28 mg, 0.291 mmol) and NaBH(OAc)₃ (62 mg, 0.29 mmol) and the resulting mixture was stirred for 16 hours. The mixture was partitioned between EtOAc and H₂O, the organic layer washed with H₂O, brine and dried (MgSO₄). The reaction mixture was concentrated under reduced pressure and purified by reverse phase semi-preparative chromatography. NMR (300 MHz): 10.34 (s, 1H), 10.27 (s, 1H), 9.03 (t, 1H), 8.97 (s, 1H), 8.39 (d, 1H), 8.30 (d, 1H), 7.98-8.08 (m, 2H), 7.96 (d, 1H), 7.75 (d, 1H), 7.54-7.67 (m, 2H), 7.47 (dd, 1H), 7.27 (d, 1H), 4.76 (d, 1H), 2.20-2.33 (m, 3H), 1.76 (s, 6H); m/z 494.

### Example 75

### Tert-butyl {2-[(3-{[(5-{[3-(1-cyano-1-methylethyl)benzoyl]amino}-2-methylphenyl)-amino]carbonyl}pyrazin-2-yl)amino]ethyl}carbamate

To a solution of 3-amino-N-(5-{[3-(1-cyano-1-methylethyl)benzoyl]amino}-2-methylphenyl)pyrazine-2-carboxamide (Example 25; 40 mg, 0.097 mmol) in 0.5 ml of anhydrous THF at ambient temperature were added tert-butyl (2-oxoethyl)carbamate (46 mg, 0.291 mmol) and NaBH(OAc)₃ (62 mg, 0.29 mmol) and the resulting mixture was stirred for 16 hours. The mixture was partitioned between EtOAc and H₂O, the organic layer washed with H₂O, brine and dried (MgSO₄). The reaction mixture was concentrated under reduced pressure and used in the next step without further purification; m/z 558.

### Example 76

### 3-[(2-Aminoethyl)amino]-N-(5-{[3-(1-cyano-1-methylethyl)benzoyl]amino}-2-methylphenyl)pyrazine-2-carboxamide

Tert-butyl {2-[(3-{[(5-{[3-(1-cyano-1-methylethyl)benzoyl]amino}-2-methylphenyl)-amino]carbonyl}pyrazin-2-yl)amino]ethyl}carbamate (Example 75; 25 mg, 0.044 mmol) was dissolved in 2 ml of anhydrous MeOH and treated with 1 ml of a solution of HCl in dioxane (4M in dioxane) and the resulting mixture was stirred at 25° C. for 1 hour. Evaporation of the volatiles afforded the title compound. NMR (300 MHz): 0.35 (s, 1H), 10.22 (d, 1H), 8.54-8.84 (m, 1H), 8.19-8.42 (m, 2H), 8.04-8.09 (m, 1H), 7.93-8.00 (m, 2H), 7.89 (d, 1H), 7.71-7.84 (m, 3H), 7.53-7.68 (m, 2H), 7.46 (dd, 1H), 7.27 (dd, 1H), 3.42-3.52 (m, 2H), 2.97-3.16 (m, 2H), 2.27 (s, 3H), 1.78 (s, 6H); m/z 458.

### Example 77

### N-(5-{[3-(1-Cyano-1-methylethyl)benzoyl]amino}-2-methylphenyl)-3-[(2-hydroxyethyl)amino]pyrazine-2-carboxamide

3-[(2-{[Tert-butyl(dimethyl)silyl]oxy}ethyl)amino]-N-(5-{[3-(1-cyano-1-methylethyl)-benzoyl]amino}-2-methylphenyl)pyrazine-2-carboxamide (Method 92; 30 mg, 0.052 mmol) was dissolved in 2 ml of anhydrous THF and treated with 1 ml of a solution of TBAF (1M in THF) and the resulting mixture was stirred at 25° C. for 1 hour. The reaction mixture was concentrated under reduced pressure and purified by reverse phase semi-preparative chromatography. NMR (300 MHz): 10.35 (s, 1H), 10.17 (d, 1H), 8.34 (dd, 1H), 8.22 (dd, 1H), 8.06 (d, 1H), 7.85-7.99 (m, 2H), 7.71-7.83 (m, 1H), 7.51-7.66 (m, 2H), 7.26 (d, 1H), 3.45-3.70 (m, 2H), 3.12-3.28 (m, 2H), 2.27 (s, 3H), 1.76 (s, 6H); m/z 459.

### Example 78

### N-(5-{[3-(1-cyano-1-methylethyl)benzoyl]amino}-2-methylphenyl)-2-(3,4-dihydroxypiperidin-1-yl)-6-methylpyrimidine-4-carboxamide

To a solution of N-(5-{[3-(1-Cyano-1-methylethyl)benzoyl]amino}-2-methylphenyl)-2-(3,6-dihydropyridin-1(2H)-yl)-6-methylpyrimidine-4-carboxamide (Example 29; 150 mg, 0.3 mmol) in acetone/water (2 ml, 1:1 v/v) were added NMMO (100 mg) followed by OsO₄ (0.100 ml, 5% w/v in t-BuOH). The resulting dark solution was stirred at 25° C. for 16 hours whereupon it was quenched with 10 ml of 1N thiosulphate solution. The mixture was allowed to stir at 25° C. for 2 hours and then the aqueous layer was extensively washed with EtOAc. The combined organic extracts dried (MgSO4) and evaporation of the volatiles under reduced pressure gave a dark brown oil. Purification by reverse phase semi-preparative chromatography afforded the title compound. NMR (300 MHz): 10.30 (s, 1H), 10.09 (s, 1H), 8.19 (s, 1H), 7.99 (t, 1H), 7.89 (d, 1H), 7.67 (d, 1H), 7.46-7.59 (m, 2H), 7.21 (d, 1H), 7.03 (s, 1H), 4.47-4.67 (m, 2H), 3.63-3.90 (m, 3H), 3.40-3.61 (m, 1H), 2.35 (d, 3H), 2.22 (s, 3H), 1.69 (s, 6H), 1.45-1.60 (m, 2H); m/z 551.

### Example 79

### N-(5-{[3-(1-Cyano-1-methylethyl)benzoyl]amino}-2-methylphenyl)-6-(dimethylamino)pyridine-2-carboxamide

6-Bromo-N-(5-{[3-(1-cyano-1-methylethyl)benzoyl]amino}-2-methylphenyl)pyridine-2-carboxamide (Example 83; 0.080 g, 0.18 mmol) in 2 M dimethylamine in THF (6 ml) and MeOH (3 ml) was heated at 65° C. in a sealed tube for 15 hours. The reaction mixture was concentrated under reduced pressure and purified by reverse phase semi-preparative chromatography. NMR (300 MHz): 10.30 (s, 1H), 10.16 (s, 1H), 8.49 (d, 1H), 8.00 (t, 1H), 7.86-7.93 (m, 1H), 7.63-7.72 (m, 2H), 7.47-7.58 (m, 2H), 7.31 (d, 1H), 7.19 (d, 1H), 6.89 (d, 1H), 3.08 (s, 6H), 2.29 (s, 3H), 1.69 (s, 6H); m/z 442.

### Example 80

The following compound was prepared by the procedure of Example 79, using the appropriate amine (commercially available unless otherwise indicated) and 6-bromo-N-(5-{[3-(1-cyano-1-methylethyl)benzoyl]amino}-2-methylphenyl)pyridine-2-carboxamide (Example 83) as starting materials.

| Ex | Compound | NMR | m/z | SM |
|---|---|---|---|---|
| 80 | N-(5-{[3-(1-Cyano-1- | 10.30(s, 1H), 10.03(s, 1H), | 497 | 1-Methyl |
| | methylethyl) | 8.20(d, 1H), 7.99(t, 1H), 7.89(d, 1H), | | piperazine |
| | benzoyl]amino}-2- | 7.80(d, 1H), 7.64-7.72(m, 1H), | | |
| | methylphenyl)-6-(4- | 7.49-7.58(m, 2H), 7.46(d, 1H), | | |
| | methyl piperazin-1- | 7.20(t, 2H), 4.51-4.62(m, 2H), | | |
| | yl) pyridine-2-carboxamide | 3.44-3.50(m, 2H), 2.98-3.22(m, | | |
| | | 4H), 2.80(d, 3H), 2.23(s, 3H), | | |
| | | 1.69(s, 6H) | | |

### Example 81

### 5-Bromo-N-(5-{[3-(1-cyano-1-methylethylbenzoyl]amino}-2-methylphenyl)-2-morpholin-4-ylpyrimidine-4-carboxamide

To a solution of 5-bromo-N-(5-{[3-(1-cyano-1-methylethyl)benzoyl]amino}-2-methylphenyl)-2-(methylthio)pyrimidine-4-carboxamide (Example 82; 902 mg, 1.72 mmol) in 5.8 ml of DCM at 0° C. was added mCPBA (77%, 0.710 mg, 3.16 mmol) and the cloudy solution was stirred at 0° C. for 30 min. Morpholine (0.15 ml, 1.72 mmol) was then added over 10 min and the resulting mixture was warmed to 25° C. and stirred for 12 hours. The reaction mixture was partitioned between EtOAc and water and the organic layer washed with H₂O, saturated aqueous NaHCO₃, brine and dried (MgSO₄). Evaporation of the solvent under reduced pressure afforded the tile compound pure enough to be used in the next step without any further purification.

### Example 82

### 5-Bromo-N-(5-{[3-(1-cyano-1-methylethyl)benzoyl]amino}-2-methylphenyl)-2-(methylthio)pyrimidine-4-carboxamide

N-(3-Amino-4-methylphenyl)-3-(1-cyano-1-methylethyl)benzamide (Method 59; 0.423 g, 1.44 mmol), 5-bromo-2-(methylthio)pyrimidine-4-carboxylic acid (0.358 g, 1.44 mmol), HATU (0.823 g, 2.16 mmol) and DIEA (0.77 ml, 4.32 mmol) were combined in 8 ml anhydrous DMF and the reaction mixture was allowed to stir at 25° C. for 15 hours. The reaction mixture was partitioned between EtOAc and water and the organic layer washed with brine, water and dried (MgSO4). Evaporation of the volatiles under reduced pressure afforded the desired product; m/z 525.

### Example 83

### 6-Bromo-N-(5-{[3-(1-cyano-1-methylethyl)benzoyl]amino}-2-methylphenyl)pyridine-2-carboxamide

6-Bromopyridine-2-carbonyl chloride (Method 91; 0.113 g, 0.51 mmol) was added to a stirring solution of N-(3-amino-4-methylphenyl)-3-(1-cyano-1-methylethyl)benzamide (Method 59; 0.150 g, 0.51 mmol) and triethylamine (0.213 μm, 1.53 mmol) in 5 ml anhydrous DCM and the reaction mixture was stirred for 30 min. at 25° C. The reaction mixture was diluted with DCM and then washed with water, brine. The organic phase was dried with Na₂SO₄(s). The solvent was removed by reduced pressure to give the title compound that was used without further purification; m/z 478.

Preparation of Starting Materials

Method 1

### Ethyl 6-methyl-2-piperidin-1-ylpyrimidine-4-carboxylate

Methyl 2-chloro-6-methylpyrimidine-4-carboxylate (1.45 g, 7.77 mmol), piperidine (0.768 ml, 7.77 mmol) and triethylamine (3.25 ml, 13.32 mmol) were combined in anhydrous ethanol (30 ml) and the reaction mixture was allowed to stir at reflux for 20 hours. The reaction mixture was allowed to cool to 25° C. and concentrated under reduced pressure. The residue was then diluted with EtOAc and washed with water and brine. The organic layers were then dried over Na₂SO₄, filtered and concentrated. The product was purified by flash chromatography using EtOAc and hexanes; m/z 250.

Methods 2-18

The following compounds were prepared by the procedure of Method 1, using the appropriate amine (commercially available unless otherwise indicated) and ester as starting materials.

| Meth | Compound | m/z | SM |
|---|---|---|---|
| 2 | Ethyl 6-methyl-2-(tetrahydro-2H-pyran-4- | 266 | Methyl 2-chloro-6- |
| | ylamino)pyrimidine-4-carboxylate | | methylpyrimidine-4-carboxylate |
| 3 | Ethyl 6-methyl-2-(4-methylpiperazin-1- | 265 | Methyl 2-chloro-6- |
| | yl)pyrimidine-4-carboxylate | | methylpyrimidine-4-carboxylate |
| 4 | Ethyl 6-methyl-2-morpholin-4- | 252 | Methyl 2-chloro-6- |
| | ylpyrimidine-4-carboxylate | | methylpyrimidine-4-carboxylate |
| 5 | Ethyl 6-methyl-2-(1,4-oxazepan-4- | 266 | Methyl 2-chloro-6- |
| | yl)pyrimidine-4-carboxylate | | methylpyrimidine-4-carboxylate |
| 6 | Ethyl 2-[(2-methoxyethyl)(methyl) | 254 | Methyl 2-chloro-6- |
| | amino]-6-methylpyrimidine-4-carboxylate | | methylpyrimidine-4-carboxylate |
| 7 | Ethyl 6-methyl-2-(3-oxopiperazin-1- | 265 | Methyl 2-chloro-6- |
| | yl)pyrimidine-4-carboxylate | | methylpyrimidine-4-carboxylate |
| 8 | Ethyl 2-[4-(tert-butoxycarbonyl)piperazin- | 351 | Methyl 2-chloro-6- |
| | 1-yl]-6-methylpyrimidine-4-carboxylate | | methylpyrimidine-4-carboxylate |
| 9 | Ethyl 2-(4-hydroxypiperidin-1-yl)-6- | 266 | Methyl 2-chloro-6- |
| | methylpyrimidine-4-carboxylate | | methylpyrimidine-4-carboxylate |
| 10 | Ethyl 2-(2,6-dimethylmorpholin-4-yl)-6- | 280 | Methyl 2-chloro-6- |
| | methylpyrimidine-4-carboxylate | | methylpyrimidine-4-carboxylate |
| 11 | Ethyl 2-chloro-6-morpholin-4- | 272 | Methyl 2,6-dichloropyrimidine- |
| | ylpyrimidine-4-carboxylate | | 4-carboxylate |
| 12 | Ethyl 2-chloro-6-morpholin-4- | 271 | Methyl 2,6-dichloro |
| | ylisonicotinate | | isonicotinate (Method 62) |
| 13 | Ethyl 2-methyl-6-morpholin-4- | 251 | Methyl 2-chloro-6-methyl |
| | ylisonicotinate | | isonicotinate (Method 63) |
| 14 | Ethyl 2-[3-(hydroxymethyl)piperidin-1- | 279 | Piperidin-3-ylmethanol and |
| | yl]-6-methylpyrimidine-4-carboxylate | | Methyl 2-chloro-6- |
| | | | methylpyrimidine-4-carboxylate |
| 15 | Ethyl 2-[2-(hydroxymethyl)morpholin-4- | 281 | Morpholin-2-ylmethanol and |
| | yl]-6-methylpyrimidine-4-carboxylate | | Methyl 2-chloro-6- |
| | | | methylpyrimidine-4-carboxylate |
| 16 | Ethyl 2-(3-hydroxypyrrolidin-1-yl)-6- | 251 | Pyrrolidin-3-ol and Methyl 2- |
| | methylpyrimidine-4-carboxylate | | chloro-6-methylpyrimidine-4- |
| | | | carboxylate |
| 17 | Ethyl 2-(3,6-dihydropyridin-1(2H)-yl)-6- | 247 | 1,2,3,6-Tetrahydropyridine and |
| | methylpyrimidine-4-carboxylate | | Methyl 2-chloro-6- |
| | | | methylpyrimidine-4-carboxylate |
| 18 | Ethyl 2-[4-(hydroxymethyl)piperidin-1- | 279 | Methyl 2-chloro-6- |
| | yl]-6-methylpyrimidine-4-carboxylate | | methylpyrimidine-4-carboxylate |

Method 19

### 6-Methyl-2-piperidin-1-ylpyrimidine-4-carboxylic acid

To a solution of ethyl 6-methyl-2-piperidin-1-ylpyrimidine-4-carboxylate (Method 1; 0.100 g, 0.40 mmol) in 6 ml THF/MeOH/H₂O (3:2:1) was added LiOH (0.034 g, 0.80 mmol) and the reaction mixture was allowed to stir at 25° C. for 4 hours. The reaction mixture was concentrated under reduced pressure affording the title compound which was used without further purification; m/z 222.

Methods 20-49

The following compounds were prepared by the procedure of Method 19, using the appropriate alkyl ester as a starting material.

| Meth | Compound | m/z | SM |
|---|---|---|---|
| 20 | 6-Methyl-2-(tetrahydro-2H-pyran- | 238 | Ethyl 6-methyl-2-(tetrahydro-2H-pyran- |
| | 4-ylamino)pyrimidine-4- | | 4-ylamino)pyrimidine-4-carboxylate |
| | carboxylic acid | | (Method 2) |
| 21 | 6-Methyl-2-(4-methylpiperazin-1- | 237 | Ethyl 6-methyl-2-(4-methylpiperazin-1- |
| | yl)pyrimidine-4-carboxylic acid | | yl)pyrimidine-4-carboxylate (Method 3) |
| 22 | 6-Methyl-2-morpholin-4- | 224 | Ethyl 6-methyl-2-morpholin-4- |
| | ylpyrimidine-4-carboxylic acid | | ylpyrimidine-4-carboxylate (Method 4) |
| 23 | 6-Methyl-2-(1,4-oxazepan-4- | 238 | Ethyl 6-methyl-2-(1,4-oxazepan-4- |
| | yl)pyrimidine-4-carboxylic acid | | yl)pyrimidine-4-carboxylate (Method 5) |
| 24 | 2-[(2-Methoxyethyl)(methyl) | 226 | Ethyl 2-[(2-methoxyethyl)(methyl) |
| | amino]-6-methylpyrimidine-4- | | amino]-6-methylpyrimidine-4- |
| | carboxylic acid | | carboxylate (Method 6) |
| 25 | 6-Methyl-2-(3-oxopiperazin-1- | 237 | Ethyl 6-methyl-2-(3-oxopiperazin-1- |
| | yl)pyrimidine-4-carboxylic acid | | yl)pyrimidine-4-carboxylate (Method 7) |
| 26 | 2-[4-(tert-Butoxycarbonyl) | 323 | Ethyl 2-[4-(tert-butoxycarbonyl) |
| | piperazin-1-yl]-6-methyl | | piperazin-1-yl]-6-methylpyrimidine-4- |
| | pyrimidine-4-carboxylic acid | | carboxylate (Method 8) |
| 27 | 2-(4-Hydroxypiperidin-1-yl)-6- | 238 | Ethyl 2-(4-hydroxypiperidin-1-yl)-6- |
| | methylpyrimidine-4-carboxylic | | methylpyrimidine-4-carboxylate |
| | acid | | (Method 9) |
| 28 | 2-(2,6-Dimethylmorpholin-4-yl)- | 251 | Ethyl 2-(2,6-dimethylmorpholin-4-yl)-6- |
| | 6-methylpyrimidine-4-carboxylic | | methylpyrimidine-4-carboxylate |
| | acid | | (Method 10) |
| 29 | 2-Chloro-6-morpholin-4- | 244 | Ethyl 2-chloro-6-morpholin-4- |
| | ylpyrimidine-4-carboxylic acid | | ylpyrimidine-4-carboxylate (Method 11) |
| 30 | 2-Chloro-6-morpholin-4- | 243 | Ethyl 2-chloro-6-morpholin-4- |
| | ylisonicotinic acid | | ylisonicotinate (Method 12) |
| 31 | 2-Methyl-6-morpholin-4- | 223 | Ethyl 2-methyl-6-morpholin-4- |
| | ylisonicotinic acid | | ylisonicotinate(Method 13) |
| 32 | 5-Chloro-2-morpholin-4- | 244 | Methyl 5-chloro-2-morpholin-4- |
| | ylpyrimidine-4-carboxylic acid | | ylpyrimidine-4-carboxylate (Method 64) |
| 33 | 4-Methyl-2-morpholin-4- | 224 | Methyl 4-methyl-2-morpholino- |
| | ylpyrimidine-5-carboxylic acid | | pyrimidine-5-carboxylate (Method 73) |
| 34 | 2-Morpholin-4-yl-4- | 278 | Methyl 2-morpholin-4-yl-4- |
| | (trifluoromethyl)pyrimidine-5- | | (trifluoromethyl)pyrimidine-5- |
| | carboxylic acid | | carboxylate (Method 74) |
| 35 | 6-{[(1R,2S)-2-(Hydroxymethyl) | 294 | Ethyl 6-{[(1R,2S)-2- |
| | cyclopropyl]amino}-2-morpholin- | | (hydroxymethyl)cyclopropyl]amino}-2- |
| | 4-ylpyrimidine-4-carboxylic acid | | morpholin-4-ylpyrimidine-4-carboxylate |
| | | | (Method 83) |
| 36 | 6-{[2-(Dimethylamino)ethyl] | 295 | Ethyl 6-{[2-(dimethylamino)ethyl] |
| | amino}-2-morpholin-4- | | amino}-2-morpholin-4-ylpyrimidine-4- |
| | ylpyrimidine-4-carboxylic acid | | carboxylate (Method 82) |
| 37 | 2-[4-(Hydroxymethyl)piperidin-1- | 251 | Ethyl 2-[4-(hydroxymethyl)piperidin-1- |
| | yl]-6-methylpyrimidine-4- | | yl]-6-methylpyrimidine-4-carboxylate |
| | carboxylic acid | | (Method 18) |
| 38 | 6-(Methylamino)-2-morpholin-4- | 238 | Ethyl 6-(methylamino)-2-morpholin-4- |
| | ylpyrimidine-4-carboxylic acid | | ylpyrimidine-4-carboxylate (Method 80) |
| 39 | 6-[(2-Hydroxyethyl)(methyl) | 282 | Ethyl 6-[(2-hydroxyethyl)(methyl) |
| | amino]-2-morpholin-4- | | amino]-2-morpholin-4-ylpyrimidine-4- |
| | ylpyrimidine-4-carboxylic acid | | carboxylate (Method 79) |
| 40 | 2-Morpholin-4-yl-6-piperazin-1- | 293 | Ethyl 2-morpholin-4-yl-6-piperazin-1- |
| | ylpyrimidine-4-carboxylic acid | | ylpyrimidine-4-carboxylate (Method 78) |
| 41 | 2-Morpholin-4-yl-6-(piperidin-4- | 307 | Ethyl 2-morpholin-4-yl-6-(piperidin-4- |
| | ylamino)pyrimidine-4-carboxylic | | ylamino)pyrimidine-4-carboxylate |
| | acid | | (Method 81) |
| 42 | 2-[3-(Hydroxymethyl)piperidin-1- | 251 | Ethyl 2-[3-(hydroxymethyl)piperidin-1- |
| | yl]-6-methylpyrimidine-4- | | yl]-6-methylpyrimidine-4-carboxylate |
| | carboxylic acid | | (Method 14) |
| 43 | 2-[2-(Hydroxymethyl)morpholin- | 253 | Ethyl 2-[2-(hydroxymethyl)morpholin- |
| | 4-yl]-6-methylpyrimidine-4- | | 4-yl]-6-methylpyrimidine-4-carboxylate |
| | carboxylic acid | | (Method 15) |
| 44 | 2-(3-Hydroxypyrrolidin-1-yl)-6- | 223 | Ethyl 2-(3-hydroxypyrrolidin-1-yl)-6- |
| | methylpyrimidine-4-carboxylic | | methylpyrimidine-4-carboxylate |
| | acid | | (Method 16) |
| 45 | 2-(3,6-Dihydropyridin-1(2H)-yl)- | 219 | Ethyl 2-(3,6-dihydropyridin-1(2H)-yl)- |
| | 6-methylpyrimidine-4-carboxylic | | 6-methylpyrimidine-4-carboxylate |
| | acid | | (Method 17) |
| 46 | 6-(Cyclopropylamino)-2- | 264 | Ethyl 6-(cyclopropylamino)-2- |
| | morpholin-4-ylpyrimidine-4- | | morpholin-4-ylpyrimidine-4-carboxylate |
| | carboxylic acid | | (Method 75) |
| 47 | 2,6-Dimorpholin-4-ylpyrimidine- | 294 | Ethyl 2,6-dimorpholin-4-ylpyrimidine- |
| | 4-carboxylic acid | | 4-carboxylate (Method 76) |
| 48 | 2-Morpholin-4-yl-6-piperidin-1- | 292 | Ethyl 2-morpholin-4-yl-6-piperidin-1- |
| | ylpyrimidine-4-carboxylic acid | | ylpyrimidine-4-carboxylate (Method 77) |
| 49 | 4-(Cyclopropylamino)-2- | 265 | Ethyl 4-(cyclopropylamino)-2- |
| | morpholin-4-ylpyrimidine-5- | | morpholin-4-ylpyrimidine-5-carboxylate |
| | carboxylic acid | | (Method 71) |

Method 50

### 3-Cyanomethylbenzoic acid methyl ester

A suspension of methyl-3-(bromomethyl)benzoate (13.5 g, 58.9 mmol) and sodium cyanide (4.33 g, 88.4 mmol) in DMF (25 ml) and water (1 ml) was stirred at 75° C. for 5 hours. The reaction mixture was quenched with water (50 ml) and extracted with EtOAc (100 ml×3). The combined organics were dried with Na₂SO₄(s) and concentrated under reduced pressure. The resulting residue was purified by column chromatography utilizing an ISCO system (hexane-EtOAc) to give 7.2 g (70%) of colourless oil. NMR (400 MHz): 7.90 (s, 1H), 7.86 (d, 1H), 7.60 (d, 1H), 7.50 (m, 1H), 4.10 (s, 2H), 3.80 (s, 3H); m/z 175.

Method 51

The following compounds were prepared by the procedure of Method 50, using the appropriate SM and sodium cyanide.

| Meth | Compound | m/z | SM |
|---|---|---|---|
| 51 | Methyl 3-(benzyloxy)-5-(cyanomethyl)benzoate | 283 | Method 88 |

Method 52

### 3-(1-Cyano-1-methylethyl)benzoic acid methyl ester

A solution of 3-cyanomethylbenzoic acid methyl ester (Method 50; 7.2 g, 41.1 mmol) in anhydrous DMSO (80 ml) was treated with sodium hydride (60%, 4.9 g, 123.3 mmol, 3 eq). Methyl iodide was then added dropwise at 0° C. The reaction mixture was stirred at 25° C. for 12 hours. The reaction mixture was then quenched with water (200 ml) and extracted with EtOAc. The combined organics were dried with Na₂SO₄(s) and concentrated under reduced pressure. The crude product was purified by column chromatography utilizing an ISCO system (hexane-EtOAc) to give 5.5 g (66%) of a colourless oil. NMR (400 MHz): 8.05 (s, 1H), 7.90 (d, 1H), 7.75 (d, 1H), 7.55 (m, 1H), 3.80 (s, 3H), 1.62 (s, 6H); m/z 203.

Method 53

The following compounds were prepared by the procedure of Method 52, using the appropriate SM and methyl iodide.

| Meth | Compound | m/z | SM |
|---|---|---|---|
| 53 | 3-Benzyloxy-5-(cyano-dimethyl-methyl)-benzoic | 310 | Method |
| | acid methyl ester | | 51 |

Method 54

### 3-(1-Cyano-1-methylethyl)benzoic acid

A solution of 3-(1-cyano-1-methylethyl)benzoic acid methyl ester (Method 52; 5.5 g, 27.1 mmol) in 100 ml of THF/MeOH/H₂O (3:1:1) was treated with lithium hydroxide (1.95 g) in 20 ml water. The mixture was stirred at 25° C. for 12 hours. The volatile solvent was removed by distillation and the resulting solution was diluted with water, then acidified with 10% HCl to pH=1-3. The resulting white solid (4.83 g, 94%) was filtered, washed with water and dried. NMR (400 MHz): 13.00 (s, 1H), 7.95 (s, 1H), 7.80 (d, 1H), 7.65 (d, 1H), 7.45 (m, 1H), 1.60 (s, 6H); m/z 189.

Methods 55-56

The following compounds were prepared by the procedure of Method 54, using the appropriate SM and lithium hydroxide.

| Meth | Compound | m/z | SM |
|---|---|---|---|
| 55 | 3-(Benzyloxy)-5-(methoxycarbonyl)benzoic | 287 | Method 87 |
| | acid | | |
| 56 | 3-(1-Cyano-1-methylethyl)-5-(2-morpholin-4- | 319 | Method 90 |
| | ylethoxy)benzoic acid | | |

Method 57

### 3-(1′-Cyano-1-methylethyl)-N-(4-methyl-3-nitro-phenyl)benzamide

A mixture of 4-methyl-3-nitroaniline (2.74 g, 18 mmol), 3-(1-cyano-1-methylethyl)benzoic acid (Method 54; 3.4 g, 18 mmol), EDCI (6.9 g, 36 mmol), HOBt (2.43 g, 18 mmol) and diisopropyl ethyl amine (3.48 g, 27 mmol) in DMF (30 ml) was stirred at 25° C. for 12 hours. The reaction mixture was diluted with DCM and then washed with water, brine. The organic phase was dried with Na₂SO₄(s). The solvent was removed by reduced pressure and the resulting product was purified by column chromatography utilizing an ISCO system (hexane-EtOAc) to give 4.4 g (53%). NMR (400 MHz): 10.50 (s, 1H), 8.40 (s, 1H), 7.40-7.95 (m, 6H), 3.20 (s, 3H), 1.65 (s, 6H); m/z 323.

Method 58

The following compound was prepared by the procedure of Method 57, using the appropriate SM.

| Meth | Compound | m/z | SM |
|---|---|---|---|
| 58 | 3-(1-Cyano-1-methylethyl)-N-(4-methyl-3- | 453 | Method 56 |
| | nitrophenyl)-5-(2-morpholin-4- | | |
| | ylethoxy)benzamide | | |

Method 59

### N-(3-Amino-4-methylphenyl)-3-(1-cyano-1-methylethyl)benzamide

A suspension of 3-(1-cyano-1-methylethyl)-N-(4-methyl-3-nitro-phenyl)benzamide (Method 57; 4 g, 13.9 mmol) and 5% palladium on carbon in hydrazine hydrate (100 ml) and ethanol (100 ml) was heated to reflux for 3 hours, then stirred at 80° C. for 12 hours. The palladium/carbon was removed by filtration and the filtrate was concentrated under reduced pressure. The residue was purified by column chromatography using an ISCO system (hexane-EtOAc) to give 3.7 g (91%) of an orange gum. 25° C. NMR (400 MHz): 9.95 (s, 1H), 8.00 (s, 1H), 7.90 (d, 1H), 7.70 (d, 1H), 7.55 (m, 1H), 7.05 (s, 1H), 6.80-6.87 (m, 2H), 4.85 (s, 2H), 2.05 (s, 3H), 1.85 (s, 6H); m/z 293.

Method 60

The following compound was prepared by the procedure of Method 59 using the appropriate SM.

| Meth | Compound | m/z | SM |
|---|---|---|---|
| 60 | N-(3-Amino-4-methylphenyl)-3-(1-cyano-1- | 423 | Method 58 |
| | methylethyl)-5-(2-morpholin-4- | | |
| | ylethoxy)benzamide | | |

Method 61

### Methyl 5-chloro-2-(methylthio)pyrimidine-4-carboxylate

To a stirring solution of 5-chloro-2-(methylthio)pyrimidine-4-carboxylic acid (3.70 g, 18.08 mmol) and DMF (10 drops) in 60 ml anhydrous DCM (60 ml) was added oxalyl chloride (7.90 ml, 90.40 mmol) and the reaction mixture was allowed to stir at 25° C. for 2 hours. The reaction was concentrated in vacuo and anhydrous methanol (20 ml) was slowly added under a nitrogen atmosphere at 0° C. The reaction mixture was then allowed to warm to 25° C. The reaction mixture was concentrated under reduced pressure to give the title compound which was used without further purification; m/z 219.

Methods 62-63

The following compounds were prepared by the procedure of Method 61, using the appropriate carboxylic acid, as a starting material.

| Meth | Compound | m/z | SM |
|---|---|---|---|
| 62 | Methyl 2,6- | 206 | 2,6- |
| | dichloroisonicotinate | | dichloroisonicotinic |
| | | | acid |
| 63 | Methyl 2-chloro-6- | 186 | 2-chloro-6- |
| | methylisonicotinate | | methylisonicotinic acid |

Method 64

### Methyl 5-chloro-2-morpholin-4-ylpyrimidine-4-carboxylate

To a stirring solution of methyl 5-chloro-2-(methylthio)pyrimidine-4-carboxylate (Method 61; 2.10 g, 9.60 mmol) in anhydrous DCM (25 ml) was added mCPBA (3.65 g, 21.12 mmol) and the reaction mixture was allowed to stir at 25° C. for 15 min. The reaction was then diluted with anhydrous dioxane (25 ml) and morpholine was added. The reaction mixture was then allowed to stir for an additional 3 hours. The reaction mixture was concentrated under reduced pressure and purified by flash chromatography (EtOAc/hexanes); m/z 258.

Method 65

### N-(4-Methyl-3-nitrophenol)-3-trifluoromethylbenzamide

A solution of 4-methyl-3-nitro-phenylamine (3.64 g, 24 mmol) and 3-trifluoromethyl-benzoyl chloride (5 g, 24 mmol) in DCM (100 ml) was treated with triethylamine (4.85 g, 48 mmol). The mixture was stirred at 25° C. for 20 min. The reaction was then quenched with water (50 ml) and stirred for 15 min. The solid was collected by vacuum filtration and washed with hexane. A second crop of solid was collected from the filtrate to give a total yield of 7.78 g (100%) of white-light yellow solid. NMR (400 MHz): 7.35 (m, 1H), 7.66 (m, 1H), 1.87 (m, 2H), 8.15 (m, 2H), 8.40 (s, 1H), 10.62 (s, 1H); m/z 324.

Method 66

### N-(3-Amino-4-methyl-phenyl)-3-trifluoromethylbenzamide

A suspension of N-(4-methyl-3-nitrophenyl)-3-trifluoromethylbenzamide (Method 65; 324 mg, 1 mmol) and tin (II) chloride (1.33 g, 7 mmol) in DMF (2 ml) was stirred at 25° C. for 12 hours. The mixture was treated with 25% of NaOH (10 ml) and extracted with chloroform (50 ml×3). The organic phases were combined and dried over anhydrous sodium sulfate and concentrated. The resulting product was purified by column chromatography utilizing an ISCO system (hexane-EtOAc) to yield 270 mg (92%) as a white solid. NMR (400 MHz): 10.00 (s, 1H), 8.05 (m, 2H), 7.80 (m, 1H), 7.60 (m, 1H), 6.92 (s, 1H), 6.70 (m, 2H), 4.70 (s, 2H), 1.87 (s, 3H); m/z 294.

Method 67

### Methyl 2-(methylamino)-6-morpholin-4-ylpyrimidine-4-carboxylate

Ethyl 2-chloro-6-morpholin-4-ylpyrimidine-4-carboxylate (Method 11; 0.340 g, 1.25 mmol) in 8 ml 2 N methyl amine in methanol was allowed to stir at 60° C. for 4 hours. The reaction mixture was concentrated under reduced pressure to give the title compound which was used without further purification. M/z 272.

Method 68

### 2-(Methylamino)-6-morpholin-4-ylpyrimidine-4-carboxylic acid

Methyl 2-(methylamino)-6-morpholin-4-ylpyrimidine-4-carboxylate (Method 67; 0.315 g, 1.25 mmol) in 40 ml 1 N aqueous KOH was allowed to stir at 110° C. for 30 min. The reaction mixture was concentrated under reduced pressure and the title compound was recrystallized from water. M/z 239.

Method 69

### 6-Methyl-2-pyridin-4-ylpyrimidine-4-carboxylic acid

To a solution of 4-pyridinyl amidine hydrochloride (0.5 g, 6.34 mmol) in 20 ml of anhydrous MeOH was added ethyl 2,4-dioxopentanoate (0.45 ml, 6.34 mmol) followed by sodium methoxide (38 ml, 19.0 mmol, 0.5 M solution in MeOH). The resulting cloudy solution was stirred for 24 hours at ambient temperature. Evaporation of the solvent afforded the title carboxylic acid; m/z 216.

Method 70

The following compound was prepared by the procedure of Method 69, using the appropriate pyridinyl amidine as a starting material.

| Meth | Compound | m/z | SM |
|---|---|---|---|
| 70 | 6-Methyl-2-pyridin-3- | 216 | 3-pyridinyl amidine |
| | ylpyrimidine-4- | | hydrochloride |
| | carboxylic acid | | |

Method 71

### Ethyl 4-(cyclopropylamino)-2-morpholin-4-ylpyrimidine-5-carboxylate

To a solution of ethyl 4-(cyclopropylamino)-2-(methylthio)pyrimidine-5-carboxylate (Method 72; residue from Method 72) in 10 ml of DCM at 0° C. was added mCPBA (60%, 3.56 g, 12.39 mmol) and the cloudy solution was stirred at this temperature for 30 min. Morpholine (0.75 ml, 8.62 mmol) was added over 10 min and the resulting mixture was warmed to room temperature and stirred for 12 hours. The reaction mixture was partitioned between EtOAc and water and the organic layer washed with H₂O, saturated aqueous NaHCO₃, brine and dried (MgSO4). Evaporation of the solvent under reduced pressure afforded the title compound which was used in the next step without any further purification.

Method 72

### Ethyl 4-(cyclopropylamino)-2-(methylthio)pyrimidine-5-carboxylate

Methyl 4-chloro-2-methylthio-pyrimidine-5-carboxylate (1.0 g, 4.3 mmol), cyclopropylamine (0.450 ml, 6.46 mmol) and triethylamine (1.8 ml, 12.9 mmol) were combined in anhydrous ethanol (5 ml) and the reaction mixture was heated to reflux for 12 hours in a sealed tube. The reaction mixture was concentrated under reduced pressure and used without any further purification in the next step.

Method 73

### Methyl 4-methyl-2-morpholino-pyrimidine-5-carboxylate

To a solution of methyl 4-methyl-2-methylthio-pyrimidine-5-carboxylate (100 mg, 0.47 mmol) in 5 ml of DCM at 0° C. was added mCPBA (60%, 407 mg, 1.41 mmol) and the cloudy solution was stirred at this temperature for 30 min. Morpholine (0.1 ml, 0.94 mmol) was added over 10 min and the resulting mixture was warmed to room temperature and stirred for 12 hours. The reaction mixture was partitioned between EtOAc and water and the organic layer washed with H₂O, saturated aqueous NaHCO₃, brine and dried (MgSO₄). Evaporation of the solvent under reduced pressure afforded methyl 4-methyl-2-morpholino-pyrimidine-5-carboxylate pure enough to be used in the next step without any further purification.

Method 74

### Methyl 2-morpholin-4-yl-4-(trifluoromethyl)pyrimidine-5-carboxylate

Methyl 2-chloro-4-trifluoromethyl-pyrimidine-5-carboxylate (1.0 g, 4.2 mmol), morpholine (0.440 ml, 5.05 mmol) and triethylamine (1.7 ml, 12.6 mmol) were combined in anhydrous ethanol (10 ml) and the reaction mixture was heated to reflux for 12 hours. The reaction mixture was concentrated under reduced pressure and used without any further purification in the next step.

Method 75

### Ethyl 6-(cyclopropylamino)-2-morpholin-4-ylpyrimidine-4-carboxylate

Methyl 2,4-dichloro-pyrimidine-6-carboxylate (1.0 g, 4.8 mmol), cyclopropylamine (0.370 ml, 5.35 mmol) were combined in anhydrous ethanol (19 ml) and the reaction mixture was allowed to stir at ambient temperature for 2 hours. The reaction mixture was concentrated under reduced pressure. The residue was redissolved in anhydrous EtOH (10 ml) and combined with morpholine (0.422 ml, 4.85 mmol) and triethylamine (1.7 ml, 9.7 mmol). The resulting mixture was heated to reflux for 16 hours. The volatiles were evaporated and the residue was then diluted with EtOAc and washed with water and brine. The organic layers were then dried (MgSO₄) filtered and concentrated. The product was purified by flash chromatography using EtOAc and hexanes; m/z 293.

Methods 76-83

The following compounds were prepared by the procedure of Method 75 using methyl 2,4-dichloro-pyrimidine-6-carboxylate, morpholine and the appropriate amine (commercially available unless otherwise indicated) as starting materials.

| Meth | Compound | m/z | SM |
|---|---|---|---|
| 76 | Ethyl 2,6-dimorpholin-4- | 322 | morpholine |
| | ylpyrimidine-4-carboxylate | | |
| 77 | Ethyl 2-morpholin-4-yl-6-piperidin- | 320 | piperidine |
| | 1-ylpyrimidine-4-carboxylate | | |
| 78 | Ethyl 2-morpholin-4-yl-6-piperazin- | 321 | piperazine |
| | 1-ylpyrimidine-4-carboxylate | | |
| 79 | Ethyl 6-[(2-hydroxyethyl)(methyl) | 310 | 2- |
| | amino]-2-morpholin-4-ylpyrimidine- | | (methyl- |
| | 4-carboxylate | | amino)ethanol |
| 80 | Ethyl 6-(methylamino)-2-morpholin- | 266 | methylamine |
| | 4-ylpyrimidine-4-carboxylate | | |
| 81 | Ethyl 2-morpholin-4-yl-6-(piperidin- | 335 | piperidin-4-amine |
| | 4-ylamino)pyrimidine-4-carboxylate | | |
| 82 | Ethyl 6-{[2-(dimethylamino)ethyl] | 323 | N,N- |
| | amino}-2-morpholin-4-ylpyrimidine- | | dimethylethane- |
| | 4-carboxylate | | 1,2-diamine |
| 83 | Ethyl 6-{[(1R,2S)-2-(hydroxymethyl) | 322 | [(1S,2R)-2- |
| | cyclopropyl]amino}-2-morpholin-4- | | aminocyclo- |
| | ylpyrimidine-4-carboxylate | | propyl]methanol |

Method 84

### 4-(Cyclopropylamino)-6-morpholin-4-ylpyridine-2-carboxylic acid

To a solution of ethyl 2,4-dichloro-pyridine-6-carboxylate (700 mg, 3.18 mmol) in 6.5 ml of absolute EtOH was added cyclopropylamine (0.22 ml, 3.18 mmol) and the resulting pale yellow solution was stirred at room temperature for 1 hour. Evaporation of the volatiles afforded the desired compound as pale yellow solid. This compound (600 mg) was then dissolved in 10 ml NMP and morpholine (0.210 ml, 2.41 mmol) was added in a microwave tube. The reaction was heated in Smith™ Personal Chemistry Microwave at 160° C. for 2200 seconds. Purification by reverse phase semi-preparative chromatography (5-95% acetonitrile/H₂O, 15 min) afforded the title compound. The ester (350 mg) was dissolved in 20 ml of wet MeOH and LiOH (100 mg) was added. The resulting mixture was stirred at room temperature for 12 hours. The precipitate was filtered and the filtrate was evaporated to afford a yellow solid, which was purified by reverse phase semi-preparative chromatography (5-20% acetonitrile/H₂O, 15 min); m/z 265

Method 85

### 6-[(Methylamino)carbonyl]nicotinic acid

To a stirring solution of 5-(methoxycarbonyl)pyridine-2-carboxylic acid (500 mg, 2.76 mmol) in 5 ml of anhydrous DMF were added sequentially HATU (1.57 g, 4.14 mmol), DIEA (1.5 ml, 8.28 mmol) and then MeNH₂ (2M in THF, 5.5 ml, 111.0 mmol). The resulting yellow solution was stirred at 25° C. for 8 hours. The reaction mixture was diluted with EtOAc and the organic layer washed with brine, H₂O, saturated aqueous NaHCO₃ and dried (MgSO₄). Evaporation of the solvents gave a yellow residue that was purified by column chromatography (Biotage® Horizons-SiO₂ column 12M-Elution 30% EtOAc-hexanes) to afford methyl 6-[(methylamino)carbonyl]nicotinate as a yellow solid (200 mg). This material was dissolved in 20 ml of MeOH and 2 ml of NaOH (50% w/w) was added. The resulting cloudy solution was heated to 70° C. for 1 hour. After removal of the solvents and adjusting the pH to about 2,6-[(methylamino)carbonyl]nicotinic acid (110 mg) was isolated as yellow solid.

Method 86

### 3-Benzyloxy-5-hydroxymethyl-benzoic acid methyl ester

A solution of 3-(benzyloxy)-5-(methoxycarbonyl)benzoic acid (Method 55; 4.5 g, 15.7 mmol) in anhydrous THF (30 ml) was treated with BH₃-dimethyl sulfide (2.0 M in THF, 9.5 ml, 19 mmol) dropwise under nitrogen at 0° C. The mixture was stirred at 0° C. for 30 min then heated up to 60° C. for 6 hours. The reaction was quenched with H₂O (5 ml) and the resulting mixture was concentrated under reduced pressure. The residue was then purified by column chromatography utilizing an ISCO system (EtOAc-Hexane) to give 3.73 g (87%) of colourless oil. NMR (400 MHz): δ 7.70 (s, 1H), 7.40-7.68 (m, 7H), 5.55 (t, 1H), 5.38 (s, 2H), 4.70 (d, 2H), 4.01 (s, 3H); m/z 273.

Method 87

### 5-Benzyloxy-isophthalic acid dimethyl ester

A solution of dimethyl 5-hydroxyisophthalate (10.5 g, 50 mmol) in 50 ml of anhydrous DMF was treated with benzyl bromide (7.3 ml, 60 mmol) dropwise. The reaction stirred for 12 hours at roomtemperature under nitrogen atmosphere. The reaction mixture was quenched with crushed ice and the resulting solid was collected by vacuum filtration. The solid washed with water and air dried to provide the desired product (14 g, 95%). NMR (400 MHz): δ 8.2 (s, 1H), 7.9 (s, 1H), 7.2-7.6 m, 5H), 7.2 (s, 1H), 5.2 (s, 2H), 3.9 (s, 6H); m/z 301.

Method 88

### 3-Benzyloxy-5-methanesulfonyloxy methyl-benzoic acid methyl ester

A solution of 3-benzyloxy-5-hydroxymethyl-benzoic acid methyl ester (Method 86; 3.73 g, 14 mmol) in anhydrous DCM (20 ml) was cooled to 0° C. To this solution, triethylamine (4.2 g, 42 mmol, 3 eq) and methane sulfonyl chloride (3.19 g, 28 mmol, 2 eq) were added respectively. The mixture was stirred at room temperature for 2 hours. The resulting salts were filtered off and washed with DCM and hexane. The filtrate was concentrated under reduced pressure and then purified by column chromatography utilizing an ISCO system (ethyl acetate-hexane) to give 3.79 g of a colourless oil as the desired product (77%). NMR (400 MHz): δ 7.12-7.40 (m, 8H), 5.05 (s, 2-H), 4.91 (s, 2H), 3.60 (s, 3H), 3.00 (s, 3H); m/z 351.

Method 89

### 3-(Cyano-dimethyl-methyl)-5-hydroxy-benzoic acid methyl ester

A suspension of 3-benzyloxy-5-(cyano-dimethyl-methyl)-benzoic acid methyl ester (Method 53; 1.7 g, 5.5 mmol) in MeOH (20 ml) was treated with 10% Pd on carbon (80 mg). The reaction was then placed on a Parr hydrogenator at 48 psi for 3 hours. The reaction mixture was then filtered through celite and the solvents were removed under reduced pressure to give a white solid 1.2 g (100%). NMR (400 MHz): δ 7.60 (s, 1H), 7.36 (s, 1H), 7.20 (s, 1H), 3.88 (s, 3H), 1.72 (s, 6H); m/z 220.

Method 90

### 3-(1-Cyano-1-methylethyl)-5-(2-morpholin-4-ylethoxybenzoic acid methyl ester

A suspension of 3-(cyano-dimethyl-methyl)-5-hydroxy-benzoic acid methyl ester (Method 89; 500 mg, 2.283 mmol), morpholinopropylchloride hydrochloride (594 mg, 2.97 mol, 1.3 eq), potassium carbonate (3.15 g, 22.8 mmol, 10 eq) and sodium iodide (35 mg, 0.23 mmol, 0.1 eq) in acetone was heated to reflux for 5 hours. The salt was filtered off, the filtrate was concentrated to provide the desired product.

Method 91

### 6-Bromopyridine-2-carbonyl chloride

Oxalyl chloride (2.46 ml, 30.3 mmol) was added to a stirring solution of 6-bromopyridine-2-carboxylic acid (0.875 g, 4.33 mmol) and DMF (3 drops) in 20 ml anhydrous DCM and the reaction mixture was stirred for 1.5 hours at 25° C. The reaction mixture was concentrated under reduced pressure to give the title compound that was used without further purification.

Method 92

### 3-[(2-{[Tert-butyl(dimethyl)silyl]oxy}ethyl)amino]-N-(5-{[3-(1-cyano-1-methylethyl)-benzoyl]amino}-2-methylphenyl)pyrazine-2-carboxamide

To a solution of 3-amino-N-(5-{[3-(1-cyano-1-methylethyl)benzoyl]amino}-2-methylphenyl)pyrazine-2-carboxamide (Example 25; 40 mg, 0.097 mmol) in 0.5 ml of anhydrous THF at ambient temperature were added TBS-protected 2-hydroxy acetaldehyde (50 mg, 0.291 mmol) and NaBH(OAc)₃ (62 mg, 0.29 mmol) and the resulting mixture was stirred for 16 hours. The mixture was partitioned between EtOAc and H₂O, the organic layer washed with H₂O, brine and dried (MgSO₄). The reaction mixture was concentrated under reduced pressure and used in the next step without further purification; m/z 573.

1. A compound of formula (I): 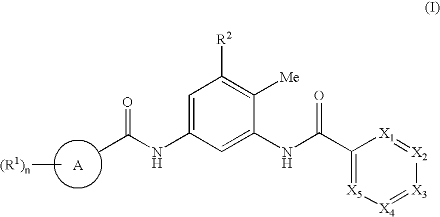
wherein:
Ring A is carbocyclyl or heterocyclyl; wherein if said heterocyclyl contains an —NH— moiety that nitrogen may be optionally substituted by a group selected from R³;
R¹ is a substituent on carbon and is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁴— or heterocyclyl-R⁵—; wherein R¹ may be optionally substituted on carbon by one or more R⁶; and wherein if said heterocyclyl contains an —NH— moiety that nitrogen may be optionally substituted by a group selected from R⁷;
R² is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁸— or heterocyclyl-R⁹—; wherein R² may be optionally substituted on carbon by one or more R¹⁰; and wherein if said heterocyclyl contains an —NH— moiety that nitrogen may be optionally substituted by a group selected from R¹¹;
X₁ is N and X₂, X₃, X₄ and X₅ are independently CR¹²; or two X₁, X₂, X₃, X₄ and X₅ are N; the other X₁, X₂, X₃, X₄ and X₅ are independently CR¹²;
n is selected from 0-4; wherein the values of R¹ may be the same or different;
R⁶ and R¹⁰ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹³— or heterocyclyl-R¹⁴—; wherein R⁶ and R¹⁰ independently of each other may be optionally substituted on carbon by one or more R¹⁵; and wherein if said heterocyclyl contains an —NH— moiety that nitrogen may be optionally substituted by a group selected from R¹⁶;
R¹² is independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R¹⁷— or heterocyclyl-R¹⁸—; wherein R¹² independently of each other may be optionally substituted on carbon by one or more R¹⁹; and wherein if said heterocyclyl contains an —NH— moiety that nitrogen may be optionally substituted by a group selected from R²⁰;
R¹⁹ is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R²¹— or heterocyclyl-R²²—; wherein R¹⁹ may be optionally substituted on carbon by one or more R²³; and wherein if said heterocyclyl contains an —NH— moiety that nitrogen may be optionally substituted by a group selected from R²⁴;
R⁴, R⁵, R⁸, R⁹, R¹³, R¹⁴, R¹⁷, R¹⁸, R²¹ and R²² are independently selected from a direct bond, —O—, —N(R²⁵)—, —C(O)—, —N(R²⁶)C(O)—, —C(O)N(R²⁷)—, —S(O)ₛ—, —SO₂N(R²⁸)— or —N(R²⁹)SO₂—; wherein R²⁵, R²⁶, R²⁷, R²⁸ and R²⁹ are independently selected from hydrogen or C₁₋₆alkyl and s is 0-2;
R³, R⁷, R¹¹, R¹⁶, R²⁰ and R²⁴ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R¹⁵ and R²³ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt thereof;
with the proviso that said compound is not 4-amino-2-(methylthio)-N-(2-methyl-5-{[3-(trifluoromethyl)benzoyl]amino}phenyl)pyrimidine-5-carboxamide.

2. A compound of formula (I), or a pharmaceutically acceptable salt thereof, as claimed in claim 1 wherein Ring A is phenyl.

3. A compound of formula (I), or a pharmaceutically acceptable salt thereof, as claimed in claim 1 wherein R¹ is a substituent on carbon and is selected from C₁₋₆alkyl or C₁₋₆alkoxy; wherein R¹ may be optionally substituted on carbon by one or more R⁶; wherein R⁵ is selected from halo, cyano or heterocyclyl-R¹⁴—; and R¹⁴ is a direct bond.

4. A compound of formula (I), or a pharmaceutically acceptable salt thereof, as claimed in claim 1 wherein R² is hydrogen.

5. A compound of formula (I), or a pharmaceutically acceptable salt thereof, as claimed in claim 1 wherein:
X₁ is N; the other X₂, X₃, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; or X₂ and X₅ are N; X₁, X₃ and X₅ are CR¹²;
wherein:
R¹² is independently selected from hydrogen, halo, cyano, amino, carboxy, carbamoyl, C₁₋₆alkyl, N—(C₁₋₆alkyl)amino, N,N—(C₁₋₆alkyl)₂amino, N—(C₁₋₆alkyl)carbamoyl, C₁₋₆alkylS(O)ₐ wherein a is 0, carbocyclyl-R¹⁷— or heterocyclyl-R¹³—; wherein R¹² independently of each other may be optionally substituted on carbon by one or more R¹⁹; and
wherein if said heterocyclyl contains an —NH— moiety that nitrogen may be optionally substituted by a group selected from R²⁰;
R¹⁹ is selected from halo, cyano, hydroxy, amino, C₁₋₆alkyl, C₁₋₆alkoxy, N,N—(C₁₋₆alkyl)₂amino, C₁₋₆alkoxycarbonylamino or heterocyclyl-R²²—; wherein R¹⁹ may be optionally substituted on carbon by one or more R²³;
R¹⁷, R¹ and R²² are independently selected from a direct bond, —N(R²⁵)— or —N(R²⁶)C(O)—; wherein R²⁵ and R²⁶ are independently selected from hydrogen;
R²⁰ is selected from C₁₋₆alkyl and C₁₋₆alkoxycarbonyl;
R²³ is hydroxy.

6. A compound of formula (I), or a pharmaceutically acceptable salt thereof, as claimed in claim 1 wherein n is selected from 1 or 2; wherein the values of R¹ may be the same or different.

7. A compound of formula (I) as claimed in claim 1: 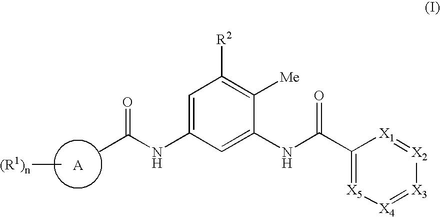
wherein:
Ring A is phenyl;
R¹ is a substituent on carbon and is trifluoromethyl, 1-cyano-1-methylethyl or 2-(morpholino)ethoxy;
R² is hydrogen;
X₁ is N; the other X₂, X₃, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²;
or X₂ and X₄ are N; X₁, X₃ and X₅ are CR¹²; or X₂ and X₅ are N; X₁, X₃ and X₅ are CR¹²;
R¹² is independently selected from hydrogen, chloro, bromo, cyano, amino, carboxy, carbamoyl, methyl, trifluoromethyl, aminomethyl, 2-(pyrrolidin-1-yl)ethyl, N-methylamino, imidazol-2-ylmethylamino, N-(2-hydroxyethyl)amino, cyclopropylamino, 2-(hydroxymethyl)cyclopropylamino, N-(2-aminoethyl)amino, N-[2-(dimethylamino)ethyl]amino, N-[2-(t-butoxycarbonylamino)ethyl]amino, N,N-dimethylamino, N-methyl-N-(2-hydroxyethyl)amino, N-methyl-N-(2-methoxyethyl)amino, methylthio, N-methylcarbamoyl, N-cyclopropylcarbamoyl, morpholino, 2,6-dimethylmorpholino, 2-(hydroxymethyl)morpholino, piperazin-4-yl, 1-methylpiperazin-4-yl, 1-(t-butoxycarbonyl)piperazin-4-yl, tetrahydropyran-4-ylamino, 2-oxopiperazin-4-yl, 1,4-oxazepan-4-yl, piperidin-1-yl, 3-(hydroxymethyl)piperidin-1-yl, 4-(hydroxymethyl)piperidin-1-yl, 4-hydroxypiperidin-1-yl, 3,4-dihydroxypiperidin-1-yl, piperidin-4-ylamino, 4-cyanoimidazol-5-ylamino, 5-oxo-2,5-dihydro-1H-pyrazol-3-ylamino, pyrazol-4-yl, 3-hydroxypyrrolidin-1-yl, 3,6-dihydropyridin-1(2H)-yl, imidazol-4-yl, pyridin-3-yl, pyridin-4-yl;
n is selected from 1 or 2; wherein the values of R¹ may be the same or different;
or a pharmaceutically acceptable salt thereof;
with the proviso that said compound is not 4-amino-2-(methylthio)-N-(2-methyl-5-{[3-(trifluoromethyl)benzoyl]amino}phenyl)pyrimidine-5-carboxamide.

8. A compound of formula (I): 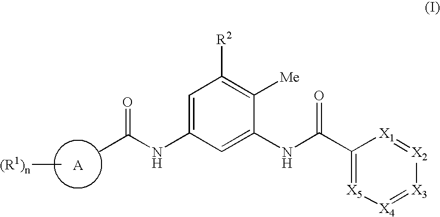
selected from:
N-(5-{[3-(1-cyano-1-methylethyl)benzoyl]amino}-2-methylphenyl)-6-(cyclopropylamino)-2-morpholin-4-ylpyrimidine-4-carboxamide;
N-(5-{[3-(1-cyano-1-methylethyl)benzoyl]amino}-2-methylphenyl)-6-morpholin-4-ylpyridine-2-carboxamide;
N-(5-{[3-(1-cyano-1-methylethyl)benzoyl]amino}-2-methylphenyl)-6-[(2-hydroxyethyl)(methyl)amino]-2-morpholin-4-ylpyrimidine-4-carboxamide;
N-(5-{[3-(1-cyano-1-methylethyl)benzoyl]amino}-2-methylphenyl)-2,6-dimorpholin-4-ylpyrimidine-4-carboxamide;
N-(5-{[3-(1-cyano-1-methylethyl)-5-(2-morpholin-4-ylethoxy)benzoyl]amino}-2-methylphenyl)-6-(cyclopropylamino)-2-morpholin-4-ylpyrimidine-4-carboxamide;
N-(5-{[3-(1-cyano-1-methylethyl)benzoyl]amino}-2-methylphenyl)-6-(methylamino)-2-morpholin-4-ylpyrimidine-4-carboxamide;
N¹-[3-(1-cyano-1-methylethyl)benzoyl]-N³-[2-(morpholino)pyrimidin-6-ylcarbonyl]-4-methylbenzene-1,3-diamine;
N¹-[3-(1-cyano-1-methylethyl)benzoyl]-N³-[2-(morpholino)-4-methylpyrimidin-6-ylcarbonyl]-4-methylbenzene-1,3-diamine; and
N¹-[3-(1-cyano-1-methylethyl)benzoyl]-N³-[2-(3-oxopiperazin-1-yl)-4-methylpyrimidin-6-ylcarbonyl]-4-methylbenzene-1,3-diamine;
or a pharmaceutically acceptable salt thereof.

9. A process for preparing a compound of formula (I), as claimed in claim 1, or a pharmaceutically acceptable salt thereof, which process, wherein variable are unless otherwise specified as defined in claim 1, comprises of:
Process a) reacting an amine of the formula (II) 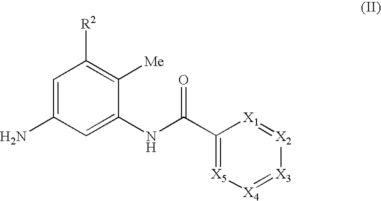
with an acid of formula (III): 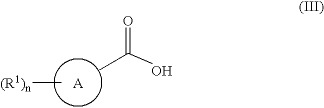
or an activated acid derivative thereof; or
Process b) reacting an amine of formula (VI): 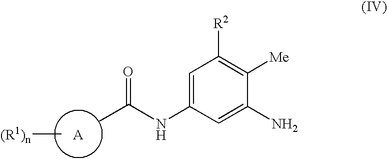
with an acid of formula (V): 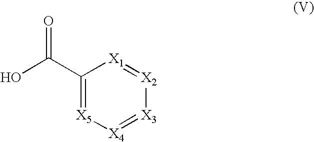
or an activated acid derivative thereof;
and thereafter if necessary:
i) converting a compound of the formula (I) into another compound of the formula (I);
ii) removing any protecting groups;
iii) forming a pharmaceutically acceptable salt.

10. A pharmaceutical composition which comprises a compound of the formula (I), or a pharmaceutically acceptable salt thereof, as claimed in claim 1, in association with a pharmaceutically-acceptable diluent or carrier.

11. (canceled)

12. A method for producing a B-Raf inhibitory effect in a warm-blooded animal such as man, in need of such treatment which comprises administering to said animal an effective amount of a compound of the formula (I): 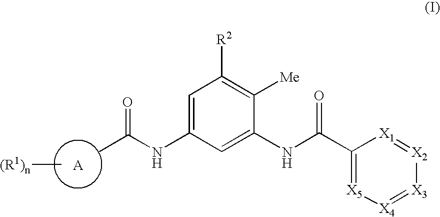
wherein:
Ring A is carbocyclyl or heterocyclyl; wherein if said heterocyclyl contains an —NH— moiety that nitrogen may be optionally substituted by a group selected from R³;
R¹ is a substituent on carbon and is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁴— or heterocyclyl-R⁵—; wherein R¹ may be optionally substituted on carbon by one or more R⁶; and wherein if said heterocyclyl contains an —NH— moiety that nitrogen may be optionally substituted by a group selected from R⁷;
R² is selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R⁸— or heterocyclyl-R⁹—; wherein R² may be optionally substituted on carbon by one or more R¹⁰; and wherein if said heterocyclyl contains an —NH— moiety that nitrogen may be optionally substituted by a group selected from R¹¹;
one or two X₁, X₂, X₃, X₄ and X₅ are N; the other X₁, X₂, X₃, X₄ and X₅ are independently CR¹²;
n is selected from 0-4; wherein the values of R¹ may be the same or different;
R⁶ and R¹⁰ are independently selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, N—(C₁₋₆alkyl)sulphamoyl, N,N—(C₁₋₆alkyl)₂sulphamoyl, C₁₋₆alkylsulphonylamino, carbocyclyl-R¹³— or heterocyclyl-R¹⁴—;
wherein R⁶ and R¹⁰ independently of each other may be optionally substituted on carbon by one or more R¹⁵; and wherein if said heterocyclyl contains an —NH— moiety that nitrogen may be optionally substituted by a group selected from R¹⁶;
R¹² is independently selected from hydrogen, halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R¹⁷— or heterocyclyl-R¹³—; wherein R¹² independently of each other may be optionally substituted on carbon by one or more R¹⁹; and wherein if said heterocyclyl contains an —NH— moiety that nitrogen may be optionally substituted by a group selected from R²⁰;
R¹⁹ is selected from halo, nitro, cyano, hydroxy, trifluoromethoxy, amino, carboxy, carbamoyl, mercapto, sulphamoyl, C₁₋₆alkyl, C₂₋₆alkenyl, C₂₋₆alkynyl, C₁₋₆alkoxy, C₁₋₆alkanoyl, C₁₋₆alkanoyloxy, N—(C₁₋₆alkyl)amino, N,N—(C₁₋₆alkyl)₂amino, C₁₋₆alkanoylamino, N—(C₁₋₆alkyl)carbamoyl, N,N—(C₁₋₆alkyl)₂carbamoyl, C₁₋₆alkylS(O)ₐ wherein a is 0 to 2, C₁₋₆alkoxycarbonyl, C₁₋₆alkoxycarbonylamino, N—(C₁₋₆alkyl)sulphamoyl, N,N—(C₁₋₆alkyl)₂sulphamoyl, C₁₋₆alkylsulphonylamino, carbocyclyl-R²¹— or heterocyclyl-R²²—; wherein R¹⁹ may be optionally substituted on carbon by one or more R²³; and wherein if said heterocyclyl contains an —NH— moiety that nitrogen may be optionally substituted by a group selected from R²⁴;
R⁴, R⁵, R⁸, R⁹, R¹³, R¹⁴, R¹⁷, R¹⁸, R²¹ and R²² are independently selected from a direct bond, —O—, —N(R²⁵)—, —C(O)—, —N(R²⁶)C(O)—, —C(O)N(R²⁷)—, —S(O)ₛ—, —SO₂N(R²⁸)— or —N(R²⁹)SO₂—; wherein R²⁵, R²⁶, R²⁷, R²⁸ and R²⁹ are independently selected from hydrogen or C₁₋₆alkyl and s is 0-2;
R³, R⁷, R¹¹, R¹⁶, R²⁰ and R²⁴ are independently selected from C₁₋₆alkyl, C₁₋₆alkanoyl, C₁₋₆alkylsulphonyl, C₁₋₆alkoxycarbonyl, carbamoyl, N—(C₁₋₆alkyl)carbamoyl, N,N—(C₁₋₆alkyl)carbamoyl, benzyl, benzyloxycarbonyl, benzoyl and phenylsulphonyl;
R¹⁵ and R²³ are independently selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
or a pharmaceutically acceptable salt thereof.

13. The method as claimed in claim 12 wherein Ring A is phenyl.

14. The method as claimed in claim 12, wherein R¹ is a substituent on carbon and is selected from C₁₋₆alkyl or C₁₋₆alkoxy; wherein R¹ may be optionally substituted on carbon by one or more R⁶; wherein R⁶ is selected from halo, cyano or heterocyclyl-R¹⁴—; and R¹⁴ is a direct bond.

15. The method as claimed in claim 12 wherein R² is hydrogen.

16. The method as claimed in claim 12 wherein X₁ is N; the other X₂, X₃, X₄ and X₅ are CR¹²; or X₂ is N; the other X₁, X₃, X₄ and X₅ are CR¹²; or X₃ is N; the other X₁, X₂, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; or X₂ and X₅ are N; X₁, X₃ and X₅ are CR¹²;
wherein:
R¹² is independently selected from hydrogen, halo, cyano, amino, carboxy, carbamoyl, C₁₋₆alkyl, N—(C₁₋₆alkyl)amino, N,N—(C₁₋₆alkyl)₂amino, N—(C₁₋₆alkyl)carbamoyl, C₁₋₆alkylS(O)ₐ wherein a is 0, carbocyclyl-R¹⁷— or heterocyclyl-R¹³—; wherein R¹² independently of each other may be optionally substituted on carbon by one or more R¹⁹; and wherein if said heterocyclyl contains an —NH— moiety that nitrogen may be optionally substituted by a group selected from R²⁰;
R¹⁹ is selected from halo, cyano, hydroxy, amino, C₁₋₆alkyl, C₁₋₆alkoxy, N,N—(C₁₋₆alkyl)₂amino, C₁₋₆alkoxycarbonylamino or heterocyclyl-R²²—; wherein R¹⁹ may be optionally substituted on carbon by one or more R²³;
R¹⁷, R¹³ and R²² are independently selected from a direct bond, —N(R²⁵)— or —N(R²⁶)C(O)—; wherein R²⁵ and R²⁶ are independently selected from hydrogen;
R²⁰ is selected from C₁₋₆alkyl and C₁₋₆alkoxycarbonyl;
R²³ is hydroxy.

17. The method as claimed in claim 12 wherein n is selected from 1 or 2; wherein the values of R¹ may be the same or different.

18. The method as claimed in claim 12
wherein:
Ring A is phenyl;
R¹ is a substituent on carbon and is trifluoromethyl, 1-cyano-1-methylethyl or 2-(morpholino)ethoxy;
R² is hydrogen;
X₁ is N; the other X₂, X₃, X₄ and X₅ are CR¹²; or X₂ is N; the other X₁, X₃, X₄ and X₅ are CR¹²; or X₃ is N; the other X₁, X₂, X₄ and X₅ are CR¹²; or X₁ and X₃ are N; X₂, X₄ and X₅ are CR¹²; or X₁ and X₄ are N; X₂, X₃ and X₅ are CR¹²; or X₁ and X₅ are N; X₂, X₃ and X₄ are CR¹²; or X₂ and X₄ are N; X₁, X₃ and X₅ are CR¹²; or X₂ and X₅ are N; X₁, X₃ and X₅ are CR¹²;
R¹² is independently selected from hydrogen, chloro, bromo, cyano, amino, carboxy, carbamoyl, methyl, trifluoromethyl, aminomethyl, 2-(pyrrolidin-1-yl)ethyl, N-methylamino, imidazol-2-ylmethylamino, N-(2-hydroxyethyl)amino, cyclopropylamino, 2-(hydroxymethyl)cyclopropylamino, N-(2-aminoethyl)amino, N-[2-(dimethylamino)ethyl]amino, N-[2-(t-butoxycarbonylamino)ethyl]amino, N,N-dimethylamino, N-methyl-N-(2-hydroxyethyl)amino, N-methyl-N-(2-methoxyethyl)amino, methylthio, N-methylcarbamoyl, N-cyclopropylcarbamoyl, morpholino, 2,6-dimethylmorpholino, 2-(hydroxymethyl)morpholino, piperazin-4-yl, 1-methylpiperazin-4-yl, 1-(t-butoxycarbonyl)piperazin-4-yl, tetrahydropyran-4-ylamino, 2-oxopiperazin-4-yl, 1,4-oxazepan-4-yl, piperidin-1-yl, 3-(hydroxymethyl)piperidin-1-yl, 4-(hydroxymethyl)piperidin-1-yl, 4-hydroxypiperidin-1-yl, 3,4-dihydroxypiperidin-1-yl, piperidin-4-ylamino, 4-cyanoimidazol-5-ylamino, 5-oxo-2,5-dihydro-1H-pyrazol-3-ylamino, pyrazol-4-yl, 3-hydroxypyrrolidin-1-yl, 3,6-dihydropyridin-1(2H)-yl, imidazol-4-yl, pyridin-3-yl, pyridin-4-yl;
n is selected from 1 or 2; wherein the values of R¹ may be the same or different;
or a pharmaceutically acceptable salt thereof;
or a pharmaceutically acceptable salt thereof.

19-21. (canceled)

22. A method for producing an anti-cancer effect in a warm-blooded animal, such as man, in need of such treatment which comprises administering to said animal an effective amount of a compound of formula (I), or a pharmaceutically acceptable salt thereof, as defined in claim 12.

23. A method of treating melanoma, papillary thyroid tumours, cholangiocarcinomas, colon cancer, ovarian cancer, lung cancer, leukaemias, lymphoid malignancies, carcinomas and sarcomas in the liver, kidney, bladder, prostate, breast and pancreas, and primary and recurrent solid tumours of the skin, colon, thyroid, lungs and ovaries, in a warm-blooded animal, such as man, in need of such treatment which comprises administering to said animal an effective amount of a compound of formula (I), as defined in claim 12.

24-26. (canceled)
